(19) United States
(12) Patent Application Publication
(10) Pub. No.: US 2024/0010641 A1
(43) Pub. Date: Jan. 11, 2024

(54) N-Linked Glycosylation Inhibitors and Methods of Using Same

(71) Applicant: YALE UNIVERSITY, New Haven, CT (US)

(72) Inventors: Joseph N. Contessa, New Haven, CT (US); Michael Van Zandt, New Haven, CT (US)

(21) Appl. No.: 18/043,436

(22) PCT Filed: Aug. 31, 2021
(86) PCT No.: PCT/US2021/048474

§ 371 Date: Sep. 22, 2023

Related U.S. Application Data
(60) Provisional application No. 63/073,312, filed on Sep. 1, 2020.

(51) Int. Cl. C07D 417/12; C07D 513/04; A61K 45/06

(52) U.S. Cl. CPC C07D 417/12; C07D 513/04; A61K 45/06

(57) ABSTRACT

The present disclosure includes compounds, compositions, and methods for preventing, ameliorating, and/or treating diseases associated with N-linked glycosylation and/or oligosaccharyltransferase function in a subject in need thereof. The methods comprise administering to the subject an effective amount of at least one compound and/or pharmaceutical composition of the disclosure.

### CROSS-REFERENCE TO RELATED APPLICATIONS

The present application claims priority under 35 U.S.C. § 119(e) to U.S. Provisional Application No. 63/073,312, filed Sep. 1, 2020, which application is incorporated herein by reference in its entirety.

### STATEMENT REGARDING FEDERALLY SPONSORED RESEARCH OR DEVELOPMENT

This invention was made with government support under GM127383 and AI134531 awarded by National Institutes of Health. The government has certain rights in the invention.

### BACKGROUND

Asparagine (N)-linked glycosylation is a co- and post-translational modification common to proteins of the endoplasmic reticulum (ER) and secretory pathway. This process requires the biosynthesis of a glycan precursor, or lipid linked oligosaccharide (LLO), and involves the coordinated function of at least 30 gene products and 17 enzymatic activities. LLO synthesis is initiated in the cytoplasm through addition of N-acetyl glucosamine to phosphorylated dolichol lipids, an enzymatic step that is blocked by the natural product tunicamycin (Tn). Sequential carbohydrate addition by glycosyltransferases associated with the cytoplasmic ER membrane elongates the LLO, and the Man₅GlcNac₂ intermediate is then transferred into the lumen of the ER by an unknown mechanism involving RFT1. Proteins that synthesize and transport carbohydrate precursors along with glycosyltransferases of the ER lumen add mannoses or glucose and form the Glc₃Man₉GlcNac₂ LLO. This mature LLO is then transferred to NXT/S/C (where X cannot be W) consensus sequences of nascent proteins by the oligosaccharyltransferase (OST).

Although the biochemical basis for synthesis and transfer of N-linked glycans to recipient proteins has been elucidated, control of this process by mammalian cells is not well understood. N-linked glycosylation was initially considered to be constitutive without sites of regulation. This belief was based on two fundamental observations: (1) many of the N-linked glycosylation genes are essential and (2) the prevalent use of tunicamycin that induces cell death. This concept, however, was incongruent with discoveries about the oligosaccharyltransferase biology. Yeast genetics demonstrated that several of the OST subunits were in fact non-essential, requiring synthetic lethal strategies for identification. Furthermore, in mammals the OST catalytic subunit (STT3 in yeast) is encoded by two separate genes, STT3A and STT3B, suggesting a mechanism for genetic regulation of LLO transfer. The subunits that compose the OST complex were also found to exist in at least 4 combinations that vary with respect to inclusion of either STT3A or STT3B and either TUSC3 or MAGT1. Thus, the OST represents at least one enzymatic node for control of N-linked glycosylation and provides molecular evidence for a model where N-linked glycosylation itself can be actively regulated.

However, attributing the consequences of abnormal N-linked glycosylation to the altered function of specific glycoproteins is difficult. The evolving experience with human congenital disorders of glycosylation, and their disparate clinical presentations, has made it difficult to identify both the specific proteins and cellular contexts that are most sensitive to disruption.

Receptor tyrosine kinase (RTK) glycoproteins such as EGFR and FGFR family members are sensitive to perturbations in glycosylation, and RTKs represent a protein class that mediates the effects of abnormal N-linked glycosylation. RTK extracellular domains are highly modified with N-linked glycans which contribute to stable conformations that facilitate ligand binding and regulate downstream signal transduction. EGFR and FGFR1, for example, have eleven and eight consensus glycosylation sites respectively, whereas the average number of N-linked sites per glycoprotein is estimated to be only 1.9. Altering the efficiency of N-glycosylation should therefore disrupt the function of RTKs. In addition other glycoproteins may be similarly affected.

Thus, there is a need in the art for novel compositions and methods that can be used to treat diseases or disorders associated with N-linked glycosylation and/or oligosaccharyl-transferase function in a mammal. The present disclosure addresses this unmet need.

### BRIEF SUMMARY

The present disclosure provides in one aspect compounds of formula (I), (II), or (III), or a salt, solvate, enantiomer, diastereomer, or tautomer thereof:

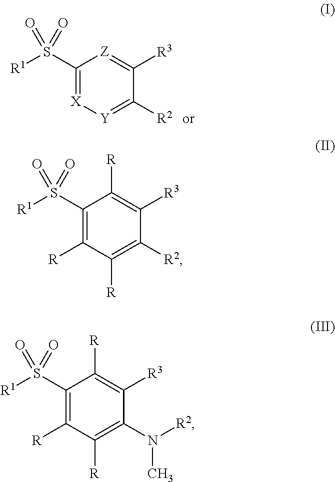

wherein each of R, R¹, R², R³, X, Y, and Z are defined elsewhere herein. In certain embodiments, the present disclosure provides a pharmaceutical composition comprising at least one compound of the present disclosure.

In another aspect, the present disclosure provides a method of inhibiting or disrupting N-linked glycosylation in a cell, the method comprising contacting the cell with an effective amount of at least one compound of the present disclosure.

In another aspect, the present disclosure provides a method of preventing, ameliorating, and/or treating a cancer in a subject in need thereof, the method comprising administering to the subject a therapeutically effective amount of at least one compound of the present disclosure.

In certain embodiments, the cancer is receptor tyrosine kinase-dependent. In certain embodiments, the cancer comprises at least one of squamous cell cancer, small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer, pancreatic cancer, glioma, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney cancer, renal cancer, prostate cancer, hepatic carcinoma, anal carcinoma, penile carcinoma, and head and neck cancer. In certain embodiments, the cancer comprises at least one of non-small cell lung cancer, small cell lung cancer, head and neck squamous cell carcinoma, breast cancer, gastric cancer, cervical cancer, colon cancer, and glioma.

### BRIEF DESCRIPTION OF THE DRAWINGS

The following detailed description of specific embodiments of the disclosure will be better understood when read in conjunction with the appended drawings. For the purpose of illustrating the disclosure, there are shown in the drawings specific embodiments. It should be understood, however, that the disclosure is not limited to the precise arrangements and instrumentalities of the embodiments shown in the drawings.

FIG. 1 illustrates Western blots of protein glycoforms after OST inhibitor treatment in vitro. HEK293 cells were treated with each inhibitor for 24 hrs at indicated concentrations. Changes in glycosylation are shown by reduced molecular weight. Examples 3 and 4 have a more potent effect consistent with their significantly improved IC₅₀ concentration (see Table 2).

FIG. 2 illustrates Western blots of protein glycoforms after OST inhibitor treatment in vitro using HEK293 cells with knockout (KO) of either STT3A or STT3B. Cells were treated with each inhibitor for 24 hrs at indicated concentrations. Changes in glycosylation are shown by reduced molecular weight. Examples 3 and 4 have a more potent effect on glycosylation in STT3B KO cells indicating enhanced specificity for STT3A. N-3563 has a more potent effect on STT3A KO cells indicating enhanced specificity for STT3B.

### DETAILED DESCRIPTION

The present disclosure relates to the unexpected discovery of novel small-molecule inhibitors of N-linked glycosylation and methods of using same.

In certain embodiments, the compounds of the present disclosure inhibit N-linked glycosylation. In other embodiments, the compounds of the disclosure inhibit oligosaccharyltransferase (OST). In yet other embodiments, the compounds of the present disclosure binds the multisubunit OST complex, reducing or inhibiting its activity. In yet other embodiments, the compounds of the present disclosure induces G1 arrest and senescence in tumor cell lines. In yet other embodiments, the compounds of the present disclosure inhibit N-linked glycosylation of proteins synthesized in vitro and/or in cell culture.

The present disclosure also relates to a method for treating or preventing a disease associated with N-linked glycosylation or OST function in a mammal by administering to the mammal a therapeutically effective amount of a N-linked glycosylation inhibitor. In certain embodiments, the disease is a tumor or cancer. In other embodiments, the disease is biliary tract carcinoma, bone cancer, tumors of the brain or nervous system, breast cancer, cervical cancer, esophageal cancer, head and neck cancer, intestinal cancer, kidney cancer, lung cancer, sarcoma, ovarian cancer, pancreatic cancer, pleural tumors, prostate cancer, skin cancer, stomach cancer, testicular tumors and cancer, thyroid cancer, urinary tract cancers, uterine cancer, adenocarcinoma, squamous cell carcinoma, glioma, or neuroendocrine tumors.

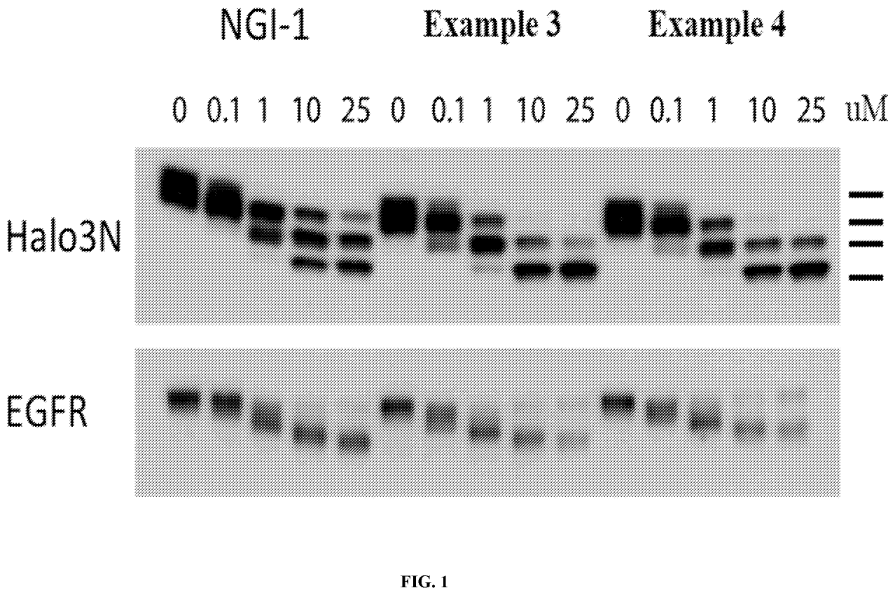

In certain embodiments, the compounds of the disclosure inhibit N-linked glycan site occupancy (FIG. 1). In certain embodiments, the compounds of the disclosure block N-linked glycosylation in mammalian cells. In certain embodiments, the compounds of the disclosure do not disrupt synthesis of lipid linked oligosaccharides. In certain embodiments, the compounds of the disclosure alter the enzymatic reaction of glycosylation per se, and cause both accumulation of mature LLOs and a marked reduction of N-linked glycosylated proteins. In certain embodiments the compounds inhibit the function of the OST. In certain embodiments, the biologic target of the compounds of the disclosure is the OST. In certain embodiments, the compounds of the disclosure reduce glycoprotein glycosylation and function. In certain embodiments the compounds cause protein misfolding and ER stress. In certain embodiments, the compounds of the disclosure alter glycoprotein trafficking. In certain embodiments the compounds cause cell cycle arrest, tumor cell death, and tumor regression.

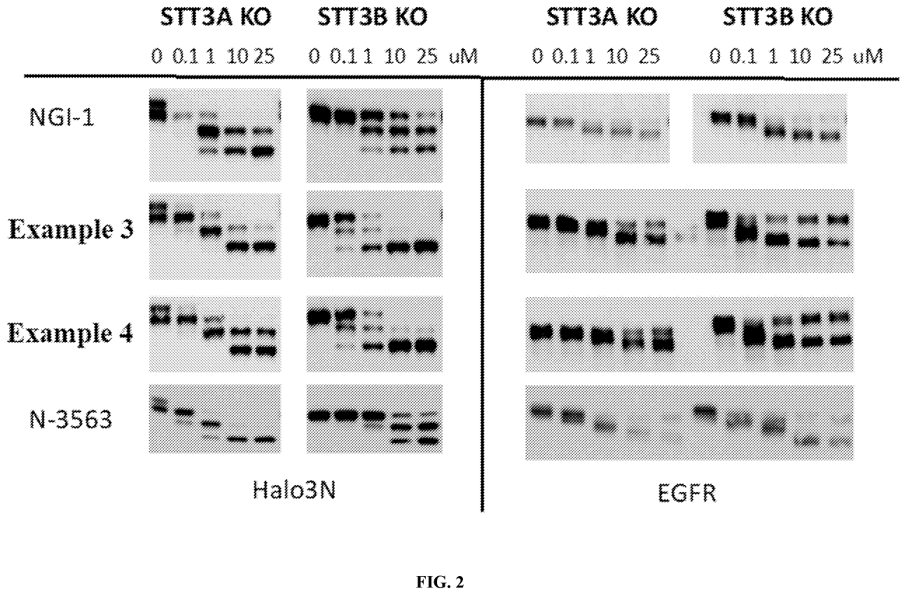

In certain embodiments, the compounds of the disclosure have a distinctive biological effect on tumor cells in comparison to Tunicamycin. In certain embodiments, the compounds of the disclosure selectively induce senescence, while tunicamycin indiscriminately causes apoptosis and cell death. Tunicamycin eliminates the synthesis of all N-linked lipid linked oligosaccharide precursors and indirectly blocks N-linked glycosylation. In certain embodiments, the compounds of the disclosure directly block the transfer of glycan precursors. While tunicamycin completely blocks the activity of a single enzymatic protein (DPAGT1), the compounds of the disclosure in certain embodiments target the multi-subunit OST complex and incompletely block its activity. In mammalian cells the OST exists in multiple isoforms and contains one of two encoded catalytic subunits (STT3A or STT3B). Catalytic subunit dependent glycosylation of specific protein consensus sequences has been defined. In certain embodiments of the disclosure catalytic subunits can be inhibited with greater specificity or selectively (FIG. 2). In yet other embodiments, the compounds of the present disclosure preferentially inhibit the STT3A subunit as compared to the STT3B subunit. In yet other embodiments, the compounds of the present disclosure preferentially inhibit the STT3B subunit as compared to the STT3A subunit.

The disclosures of WO2017/019540A2 and US2019/0000858A1 are incorporated herein in their entireties by reference.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, specific methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal”, when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

A disease or disorder is “alleviated” if the severity of a symptom of the disease or disorder, the frequency with which such a symptom is experienced by a patient, or both, is reduced.

The terms “cancer” refers to the physiological condition in a subject typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small cell lung cancer, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, as well as head and neck cancer.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount”, “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Eₘₐₓ) achieved within an assay.

As used herein, the term “OST” refers to oligosaccharyltransferase.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

The terms “patient”, “subject”, or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

A “therapeutic” treatment is a treatment administered to a subject who exhibits signs of pathology, for the purpose of diminishing or eliminating those signs.

As used herein, the term “alkyl”, by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl as defined herein, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “haloalkyl” means alkyl as defined herein, substituted by one, two or three substituents selected from the group consisting of F, Cl, Br, and I.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized or substituted. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, —NH(CH₂)ₘOH (m=1-6), —N(CH₃)(CH₂)ₘOH (m=1-6), —NH(CH₂)ₘOCH₃ (m=1-6), and —CH₂CH₂S(═O) CH₃. Up to two heteroatoms may be consecutive, such as, for example, —CH₂NH—OCH₃, or —CH₂CH₂SSCH₃

As used herein, the term “alkoxy” employed alone or in combination with other terms means, unless otherwise stated, an alkyl group having the designated number of carbon atoms, as defined herein, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In certain embodiments, the cycloalkyl group is saturated or partially unsaturated. In other embodiments, the cycloalkyl group is fused with an aromatic ring. Cycloalkyl groups include groups having from 3 to 10 ring atoms. Illustrative examples of cycloalkyl groups include, but are not limited to, the following moieties:

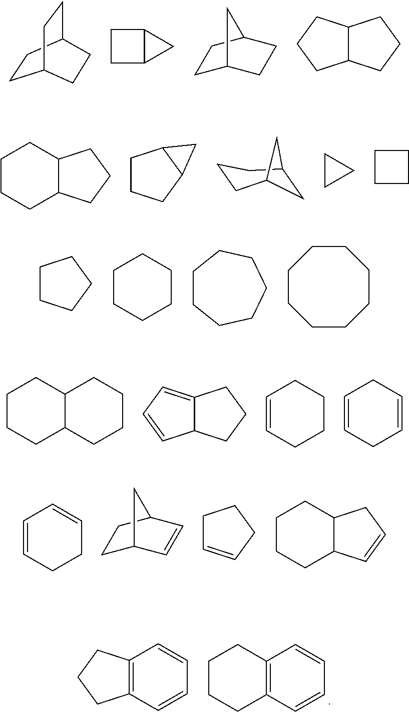

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycloalkyl” or “heterocyclyl” refers to a heteroalicyclic group containing one to four ring heteroatoms each selected from O, S and N. In certain embodiments, each heterocycloalkyl group has from 4 to 10 atoms in its ring system, with the proviso that the ring of said group does not contain two adjacent O or S atoms. In other embodiments, the heterocycloalkyl group is fused with an aromatic ring. In certain embodiments, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

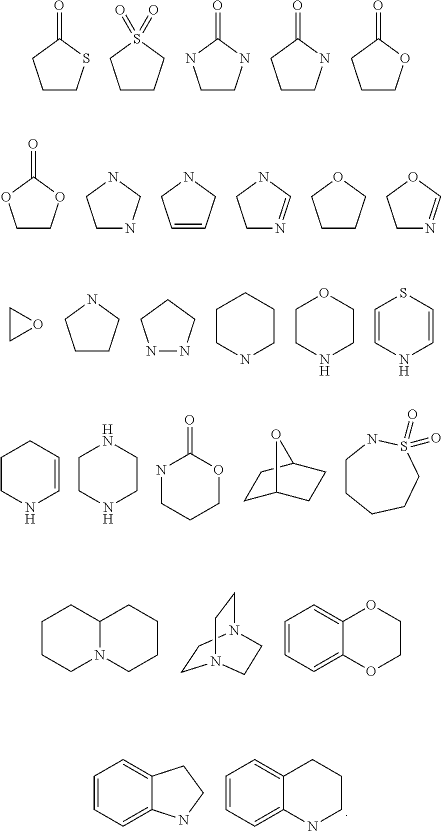

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl”, employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. Preferred examples are phenyl and naphthyl, most preferred is phenyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heteroaryl” or “heteroaromatic” refers to a heterocycle having aromatic character. A polycyclic heteroaryl may include one or more rings that are partially saturated. Examples include the following moieties:

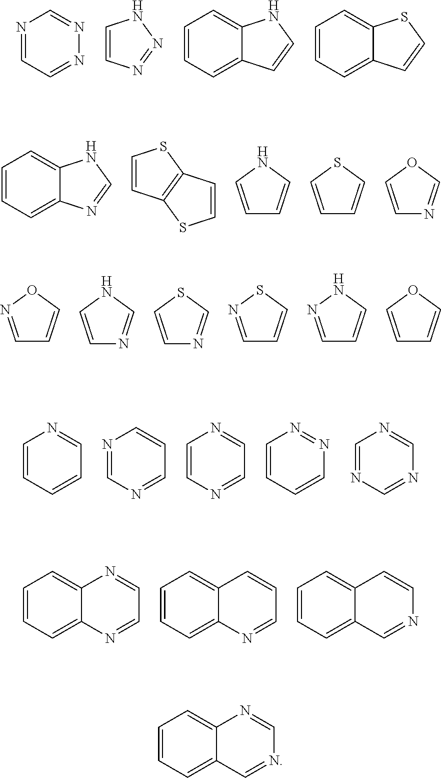

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two.

As used herein, the term “optionally substituted” means that the referenced group may be substituted or unsubstituted. In certain embodiments, the referenced group is optionally substituted with zero substituents, i.e., the referenced group is unsubstituted. In other embodiments, the referenced group is optionally substituted with one or more additional group(s) individually and independently selected from groups described herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

In certain embodiments, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In other embodiments, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet other embodiments, the substituents are independently selected from the group consisting of C₁₋₆alkyl, C₁₋₆ alkoxy, halo, acetamido, and nitro. As used herein, where a substituent is an alkyl or alkoxy group, the carbon chain may be branched, straight or cyclic, with straight being preferred.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

The compounds of the present disclosure may be synthesized using techniques well-known in the art of organic synthesis. The starting materials and intermediates required for the synthesis may be obtained from commercial sources or synthesized according to methods known to those skilled in the art.

Compounds of formulas (I)-(III) may be prepared by the general schemes described herein, using the synthetic method known by those skilled in the art. The disclosures of WO 2017/019540A2 and US 2019/0000858A1 are incorporated herein in their entireties by reference. The following examples illustrate non-limiting embodiments of the disclosure. Certain compounds are disclosed herein; a salt, solvate, enantiomer, diastereomer, and/or tautomer of any such compound is also contemplated within the disclosure.

In certain embodiments, the disclosure provides a compound of formula (I) or formula (II), or a salt, solvate, enantiomer, diastereomer, or tautomer thereof:

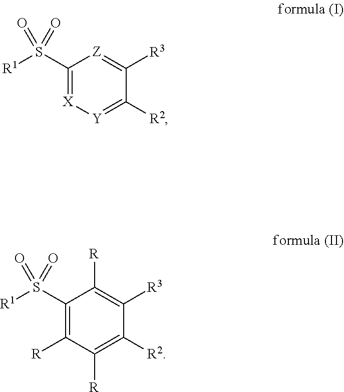

In the compound of formula (I) or formula (II):
X is CR⁴ or N;
Y is CR⁴ or N;
Z is CR⁴ or N;
   wherein at least one of X, Y, and Z is N;
each occurrence of R is independently selected from the group consisting of H, —(C₁-C₆)alkyl, —(C₃-C₆)cycloalkyl, —(C₁-C₆)haloalkyl, —(C₁-C₆)alkoxy, —(C₃-C₁₀)heterocyclyl, —(C₁-C₆)heteroalkyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl,
   wherein at least one R is not H;
   wherein each occurrence of alkyl, heteroalkyl, cycloalkyl, heterocyclyl, aryl, or heteroaryl group is independently optionally substituted;

In certain embodiments, R¹ is

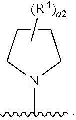

In certain embodiments, R¹ is

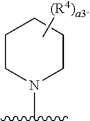

In certain embodiments, R¹ is

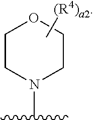

In certain embodiments, R¹ is

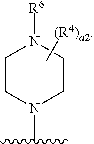

In certain embodiments, R¹ is —N(R⁴)₂.

In certain embodiments, R² is

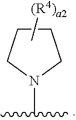

In certain embodiments, R² is

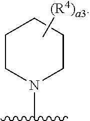

In certain embodiments, R² is

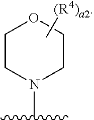

In certain embodiments, R² is

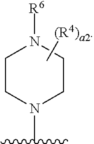

In certain embodiments, R² is

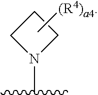

In certain embodiments, R² is

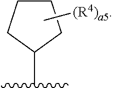

In certain embodiments, R² is —N(R⁴)₂.

In certain embodiments, R³ is

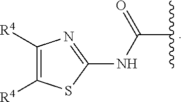

In certain embodiments, R³ is

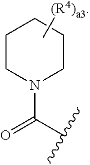

In certain embodiments, R³ is

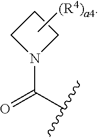

In certain embodiments, R³ is

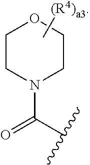

In certain embodiments, R³ is

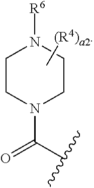

In certain embodiments, R³ is

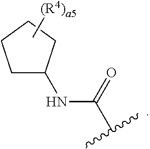

In certain embodiments, R³ is

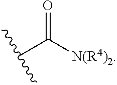

In certain embodiments, R³ is

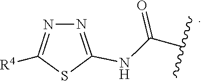

In certain embodiments, R³ is

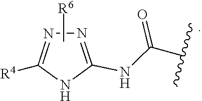

In certain embodiments, R³ is

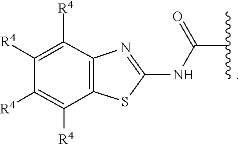

In certain embodiments, R³ is

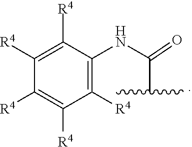

In certain embodiments, R³ is

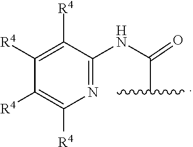

In certain embodiments, R³ is

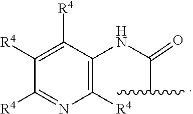

In certain embodiments, R³ is

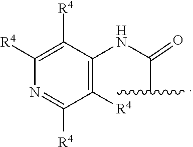

In certain embodiments, R³ is

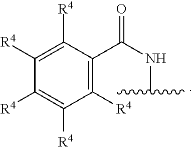

each occurrence of R⁴ is independently selected from the group consisting of H, —(C₁-C₆)alkyl, —(C₃-C₆)cycloalkyl, —(C₁-C₆)haloalkyl, —(C₁-C₆)alkoxy, —(C₁-C₆)heteroalkyl, —(C₃-C₁₀)heterocyclyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl, or two R⁴ bound to the same carbon atom or bound to adjacent carbon atoms combine to form a 3- to 10-membered cycloalkyl or heterocyclyl;
   wherein each occurrence of alkyl, heteroalkyl, cycloalkyl, heterocyclyl, aryl, or heteroaryl group is independently optionally substituted;
each occurrence of R⁵ is independently selected from the group consisting of H, —(C₁-C₆)alkyl, —(C₁-C₆)heteroalkyl, —(C₃-C₆)cycloalkyl, —(C₃-C₁₀)heterocyclyl, aryl, and —(C₅-C₁₀)heteroaryl,
   wherein each occurrence of alkyl, heteroalkyl, cycloalkyl, heterocyclyl, aryl, or heteroaryl group is independently optionally substituted;
each occurrence of R⁶ is independently selected from the group consisting of H, optionally substituted (C₁-C₆)alkyl, optionally substituted (C₁-C₆)heteroalkyl, optionally substituted (C₃-C₆)cycloalkyl, optionally substituted (C₁-C₆)acyl, and optionally substituted benzoyl;
each occurrence of a1 is independently 1, 2, or 3;
each occurrence of a2 is independently 1, 2, 3, 4, 5, 6, 7, or 8;
each occurrence of a3 is independently 1, 2, 3, 4, 5, 6, 7, 8, 9, or 10;
each occurrence of a4 is independently 1, 2, 3, 4, 5, or 6; and
each occurrence of a5 is independently 1, 2, 3, 4, 5, 6, 7, 8, or 9.

In various embodiments, in formula (I), X is N, and Y and Z are independently CR⁴. In various embodiments, in formula (I), Y is N, and X and Z are independently CR⁴. In various embodiments, in formula (I), Z is N, and X and Y are independently CR⁴. In various embodiments, in formula (I), Y and Z are N, and X is CR⁴. In various embodiments, in formula (I), X and Y are N, and Z is CR⁴. In various embodiments, in formula (I), X and Z are N, and Y is CR⁴. In various embodiments, in formula (I), X, Y, and Z are N.

In various embodiments, in formula (I), R¹ is —N(R⁴)₂ or

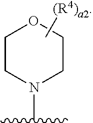

In various embodiments, in formula (I), R² is —N(R⁴)(CH₃). In various embodiments, in formula (I), at least one R⁴ in R² (such as, for example, in —N(R⁴)(CH₃)) is —CH₂-(cyclopropyl) or cyclopentyl.

In various embodiments, in formula (I), R³ is

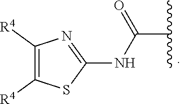

In various embodiments, in formula (I), each R⁴ in R³ is independently H or —(C₁-C₆)alkyl. In various embodiments, in formula (I), in R³ two R⁴ bound to the same carbon atom or bound to adjacent carbon atoms combine to form a 3- to 10-membered cycloalkyl or heterocyclyl.

In various embodiments, the compound of formula (I) is

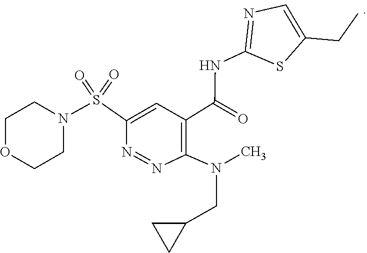 [SMILES: CCc1cnc(NC(=O)c2cc(S(=O)(=O)N3CCOCC3)nnc2N(C)CC2CC2)s1]

In various embodiments, the compound of formula (I) is

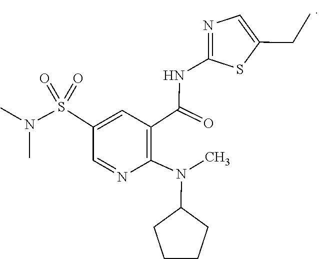 [SMILES: CCc1cnc(NC(=O)c2cc(S(=O)(=O)N(C)C)cnc2N(C)C2CCCC2)s1]

In various embodiments, the compound of formula (I) is

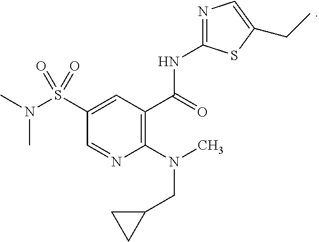 [SMILES: CCc1cnc(NC(=O)c2cc(S(=O)(=O)N(C)C)cnc2N(C)CC2CC2)s1]

In various embodiments, the compound of formula (I) is

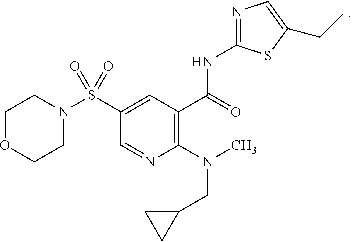 [SMILES: CCc1cnc(NC(=O)c2cc(S(=O)(=O)N3CCOCC3)cnc2N(C)CC2CC2)s1]

In various embodiments, the compound of formula (I) is

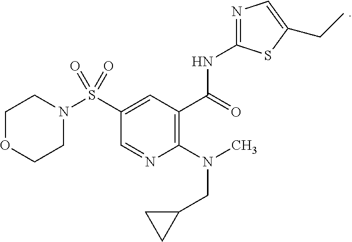 [SMILES: CCc1cnc(NC(=O)c2cc(S(=O)(=O)N3CCOCC3)cnc2N(C)CC2CC2)s1]

In various embodiments, the compound of formula (I) is

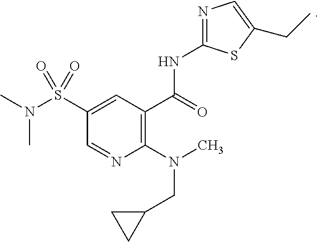 [SMILES: CCc1cnc(NC(=O)c2cc(S(=O)(=O)N(C)C)cnc2N(C)CC2CC2)s1]

In various embodiments, the compound of formula (I) is

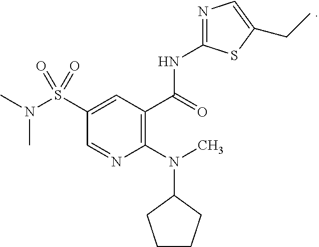 [SMILES: CCc1cnc(NC(=O)c2cc(S(=O)(=O)N(C)C)cnc2N(C)C2CCCC2)s1]

In various embodiments, the compound of formula (I) is

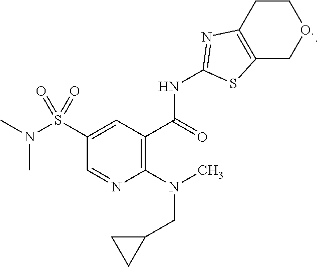 [SMILES: CN(CC1CC1)c1ncc(S(=O)(=O)N(C)C)cc1C(=O)Nc1nc2c(s1)COCC2]

In various embodiments, in formula (II), at least one R is F, Cl, Br, or I. In various embodiments, in formula (II), at least one R is F. In various embodiments, in formula (II), one R is F, Cl, Br, or I, and the remaining R groups are H. In various embodiments, in formula (II), one R is F, and the remaining R groups are H.

In various embodiments, in formula (II), R¹ is N(R⁴)₂ or

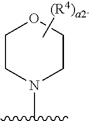

In various embodiments, in formula (II), R² is N(R⁴)(CH₃). In various embodiments, in formula (II), R²

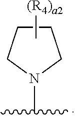

In various embodiments, in formula (II), R² is

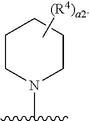

In various embodiments, in formula (II), R² is

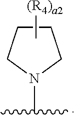

In various embodiments, in formula (II), at least one R⁴ in R² (such as, for example, in —N(R⁴)(CH₃)) is —CH₂-(cyclopropyl) or cyclopentyl.

In various embodiments, in formula (II), R³ is

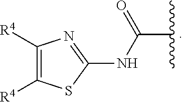

In various embodiments, in formula (II), each R⁴ in R³ is independently H or —(C₁-C₆)alkyl.

In various embodiments, the compound of formula (II) is

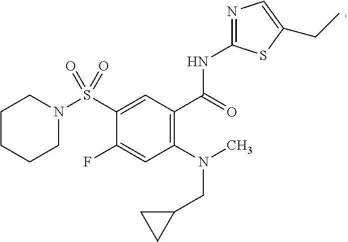 [SMILES: CCc1cnc(NC(=O)c2cc(S(=O)(=O)N3CCCCC3)c(F)cc2N(C)CC2CC2)s1]

In various embodiments, the compound of formula (II) is

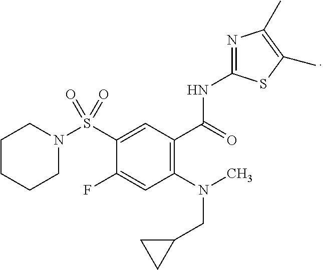 [SMILES: Cc1nc(NC(=O)c2cc(S(=O)(=O)N3CCCCC3)c(F)cc2N(C)CC2CC2)sc1C]

In various embodiments, the compound of formula (II) is

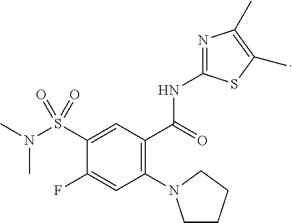

In various embodiments, the compound of formula (II) is and

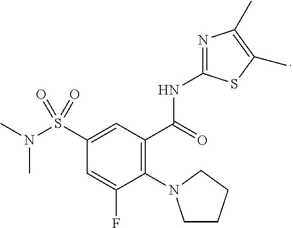

In certain embodiments, in formula (II), R² is

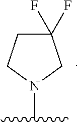 [SMILES: *N1CCC(F)(F)C1]

In various embodiments, in formula (II), R³ is

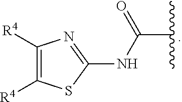

In various embodiments, in formula (II), R³ is

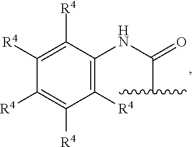

In various embodiments, the compound of formula (II) is

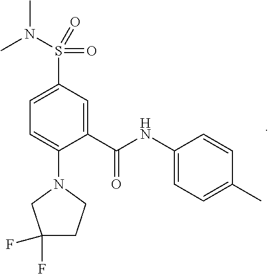

In various embodiments, the compound of formula (II) is

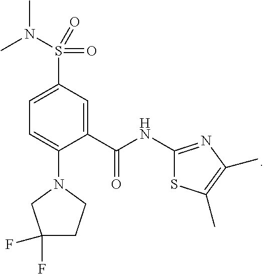

In various embodiments, the compound of formula (II) is

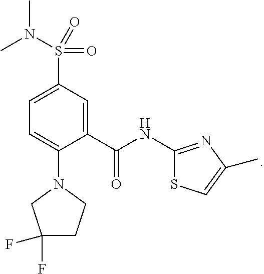

In various embodiments, the compound of formula (II) is

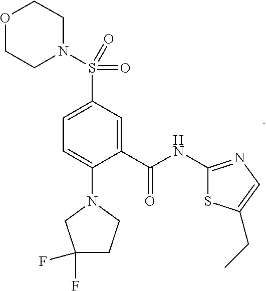 [SMILES: CCc1cnc(NC(=O)c2cc(S(=O)(=O)N3CCOCC3)ccc2N2CCC(F)(F)C2)s1]

In various embodiments, the compound of formula (II) is

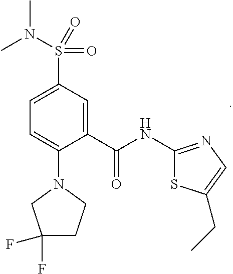 [SMILES: CCc1cnc(NC(=O)c2cc(S(=O)(=O)N(C)C)ccc2N2CCC(F)(F)C2)s1]

In various embodiments, the compound of formula (II) is

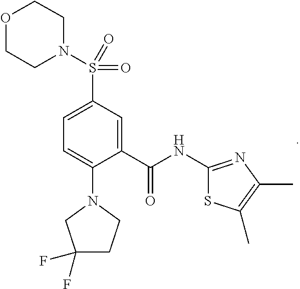 [SMILES: Cc1nc(NC(=O)c2cc(S(=O)(=O)N3CCOCC3)ccc2N2CCC(F)(F)C2)sc1C]

In various embodiments, the compounds preferentially inhibits the STT3B subunit as compared to the STT3A subunit.

In a certain embodiment, the disclosure provides a compound of formula (III), or a salt, solvate, enantiomer, diastereomer or tautomer thereof:

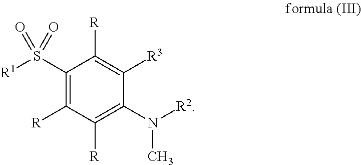

In the compound of formula (III):
each occurrence of R is independently selected from the group consisting of H, —(C₁-C₆)alkyl, —(C₃-C₆)cycloalkyl, —(C₁-C₆)haloalkyl, —(C₁-C₆)alkoxy, —(C₃-C₁₀)heterocyclyl, —(C₁-C₆)heteroalkyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl,
   wherein the alkyl, heteroalkyl, cycloalkyl, heterocyclyl, aryl, or heteroaryl group is optionally substituted;
R¹ is selected from the group consisting of

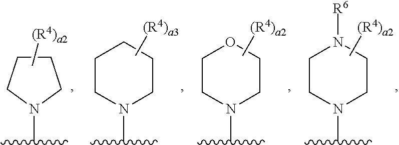

and —N(R⁴)₂;
R² is selected from the group consisting of —(C₁-C₆)alkyl, —(C₃-C₆)cycloalkyl, —(C₁-C₆)haloalkyl, —(C₃-C₁₀)heterocyclyl, —(C₁-C₆)heteroalkyl, aryl, —CH₂-aryl, and —(C₅-C₁₀)heteroaryl,
   wherein the alkyl, heteroalkyl, cycloalkyl, heterocyclyl, aryl, or heteroaryl group is optionally substituted;
R³ is selected from the group consisting of:

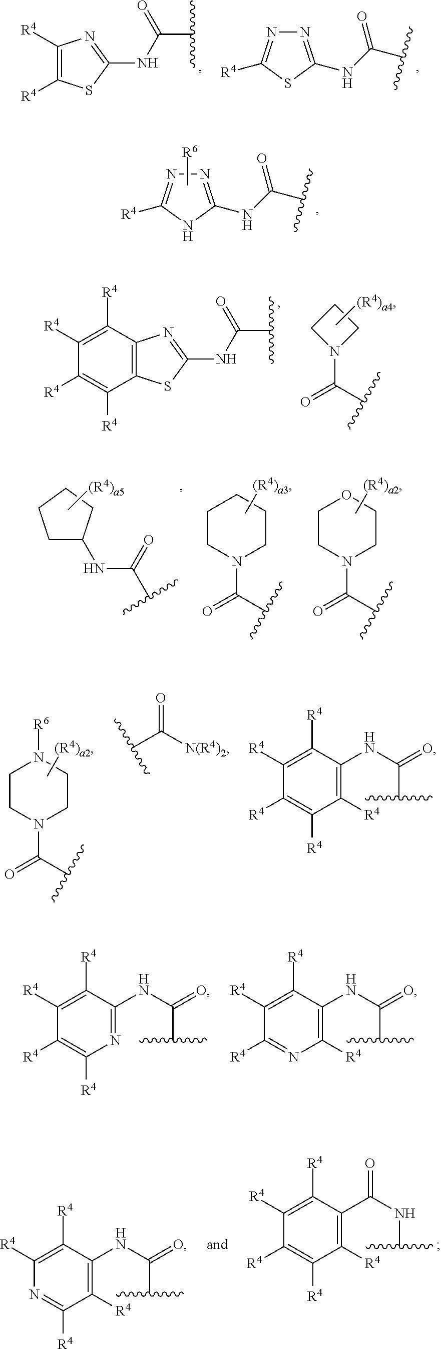

each occurrence of R⁴ is independently selected from the group consisting of H, —(C₁-C₆)alkyl, —(C₃-C₆)cycloalkyl, —(C₁-C₆)haloalkyl, —(C₁-C₆)alkoxy, —(C₃-C₁₀)heterocyclyl, —(C₁-C₆)heteroalkyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl, or two R⁴ bound to the same carbon atom or bound to adjacent carbon atoms combine to form a 3- to 10-membered cycloalkyl or heterocyclyl;
   wherein the alkyl, heteroalkyl, cycloalkyl, heterocyclyl, aryl, or heteroaryl group is optionally substituted;
each occurrence of R⁵ is independently selected from the group consisting of H, —(C₁-C₆)alkyl, —(C₁-C₆)heteroalkyl, —(C₃-C₆)cycloalkyl, —(C₃-C₁₀)heterocyclyl, aryl, and —(C₅-C₁₀)heteroaryl,
   wherein the alkyl, heteroalkyl, cycloalkyl, heterocyclyl, aryl, or heteroaryl group is optionally substituted;
each occurrence of R⁶ is independently selected from the group consisting of H, optionally substituted (C₁-C₆)alkyl, optionally substituted (C₁-C₆)heteroalkyl, optionally substituted (C₃-C₆)cycloalkyl, optionally substituted (C₁-C₆)acyl, and optionally substituted benzoyl.
each occurrence of a1 is independently 1, 2, or 3;
each occurrence of a2 is independently 1, 2, 3, 4, 5, 6, 7, or 8;
each occurrence of a3 is independently 1, 2, 3, 4, 5, 6, 7, 8, 9, or 10;
each occurrence of a4 is independently 1, 2, 3, 4, 5, or 6;
each occurrence of a5 is independently 1, 2, 3, 4, 5, 6, 7, 8, or 9;
   with the proviso that the compound is not 2-(dimethylamino)-5-(dimethylsulfamoyl)-N-(5-methyl-1,3-thiazol-2-yl)benzamide.

In various embodiments, in formula (III), R¹ is N(R⁴)₂ or

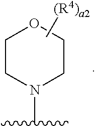

In various embodiments, in formula (III), each occurrence of R⁴ in R¹ is independently hydrogen or methyl.

In various embodiments, in formula (III), R² is —CH₂-(cyclopropyl), —CH₂-(cyclobutyl), —CH₂-(cyclopentyl), —CH₂-(cyclohexyl), cyclobutyl, cyclopentyl, cyclohexyl, ethyl, isopropyl, isobutyl, or allyl.

In various embodiments, in formula (III), R³ is

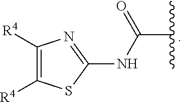

In various embodiments, in formula (III), each occurrence of R⁴ in R³ is independently H or —(C₁-C₆)alkyl.
In various embodiments, the compound of formula (III) is

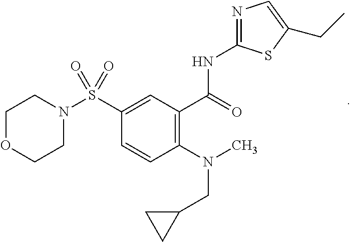 [SMILES: CCc1cnc(NC(=O)c2cc(S(=O)(=O)N3CCOCC3)ccc2N(C)CC2CC2)s1]

In various embodiments, the compound of formula (III) is

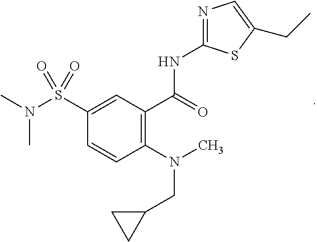 [SMILES: CCc1cnc(NC(=O)c2cc(S(=O)(=O)N(C)C)ccc2N(C)CC2CC2)s1]

In various embodiments, the compound of formula (III) is

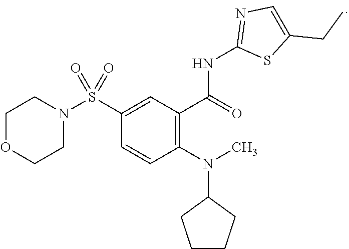 [SMILES: CCc1cnc(NC(=O)c2cc(S(=O)(=O)N3CCOCC3)ccc2N(C)C2CCCC2)s1]

various embodiments, the compound of formula (III) is

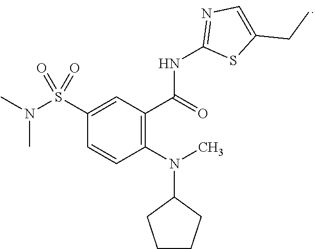 [SMILES: CCc1cnc(NC(=O)c2cc(S(=O)(=O)N(C)C)ccc2N(C)C2CCCC2)s1]

In various embodiments, the compound of formula (III) is

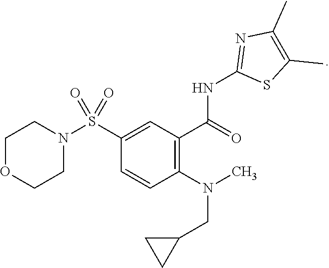 [SMILES: Cc1nc(NC(=O)c2cc(S(=O)(=O)N3CCOCC3)ccc2N(C)CC2CC2)sc1C]

In various embodiments, the compound of formula (III) is

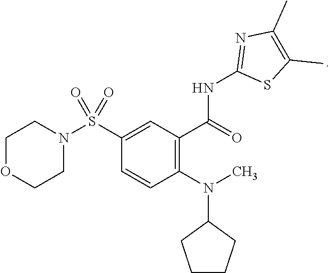 [SMILES: Cc1nc(NC(=O)c2cc(S(=O)(=O)N3CCOCC3)ccc2N(C)C2CCCC2)sc1C]

In various embodiments, the compound of formula (III) is

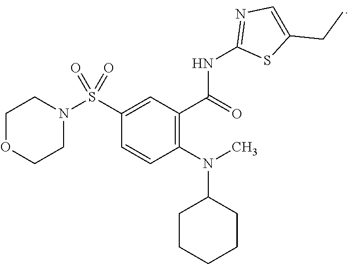 [SMILES: CCc1cnc(NC(=O)c2cc(S(=O)(=O)N3CCOCC3)ccc2N(C)C2CCCCC2)s1]

In various embodiments, the compound of formula (III) is

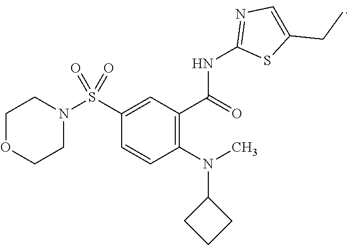 [SMILES: CCc1cnc(NC(=O)c2cc(S(=O)(=O)N3CCOCC3)ccc2N(C)C2CCC2)s1]

In various embodiments, the compound of formula (III) is

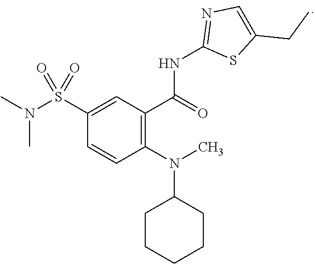 [SMILES: CCc1cnc(NC(=O)c2cc(S(=O)(=O)N(C)C)ccc2N(C)C2CCCCC2)s1]

In various embodiments, the compound of formula (III) is

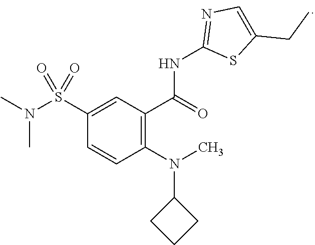 [SMILES: CCc1cnc(NC(=O)c2cc(S(=O)(=O)N(C)C)ccc2N(C)C2CCC2)s1]

In various embodiments, the compound of formula (III) is

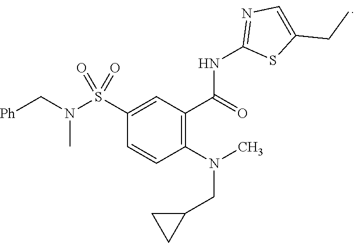 [SMILES: CCc1cnc(NC(=O)c2cc(S(=O)(=O)N(C)Cc3ccccc3)ccc2N(C)CC2CC2)s1]

In various embodiments, the compound of formula (III) is

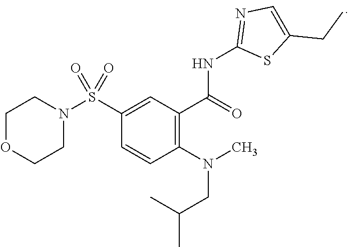 [SMILES: CCc1cnc(NC(=O)c2cc(S(=O)(=O)N3CCOCC3)ccc2N(C)CC(C)C)s1]

In various embodiments, the compound of formula (III) is

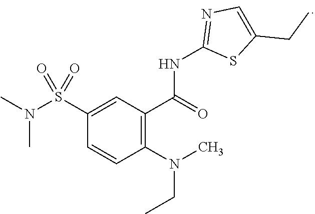 [SMILES: CCc1cnc(NC(=O)c2cc(S(=O)(=O)N(C)C)ccc2N(C)CC)s1]

In various embodiments, the compound of formula (III) is

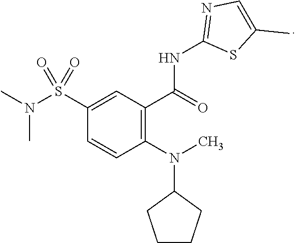

In various embodiments, the compound of formula (III) is

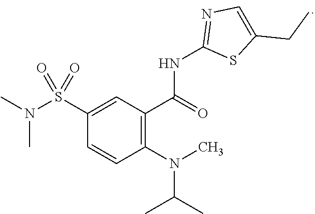 [SMILES: CCc1cnc(NC(=O)c2cc(S(=O)(=O)N(C)C)ccc2N(C)C(C)C)s1]

In various embodiments, the compound of formula (III) is

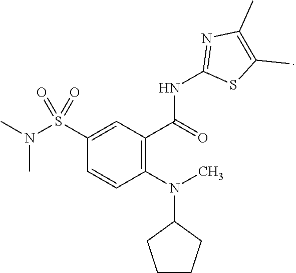

In various embodiments, the compound of formula (III) is

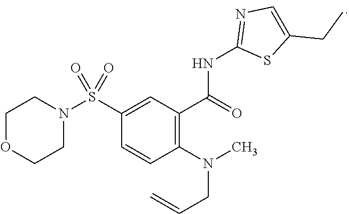 [SMILES: C=CCN(C)c1ccc(S(=O)(=O)N2CCOCC2)cc1C(=O)Nc1ncc(CC)s1]

In various embodiments, the compound of formula (III) is

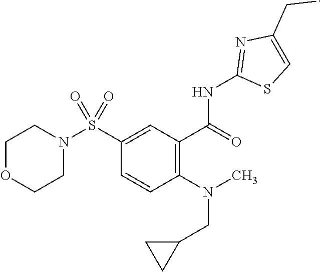

In various embodiments, the compound of formula (III) is

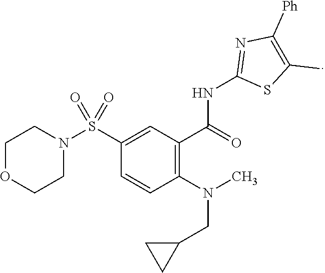

In various embodiments, the compound of formula (III) is

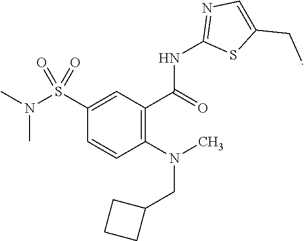

In certain embodiments, the compounds of the present disclosure preferentially inhibit the STT3A subunit as compared to the STT3B subunit.

In certain embodiments, the compounds of the present disclosure preferentially inhibit the STT3B subunit as compared to the STT3A subunit.

The compositions described herein include a pharmaceutical composition that includes at least one pharmaceutically acceptable carrier and at least one agent of formula (I), formula (II), or formula (III). The pharmaceutical compositions described herein can further include at least one additional therapeutic compound that treats or prevents cancer.

The compounds of the disclosure may possess one or more stereocenters, and each stereocenter may exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the disclosure may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the disclosure are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

### Methods of Synthesizing Compounds

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

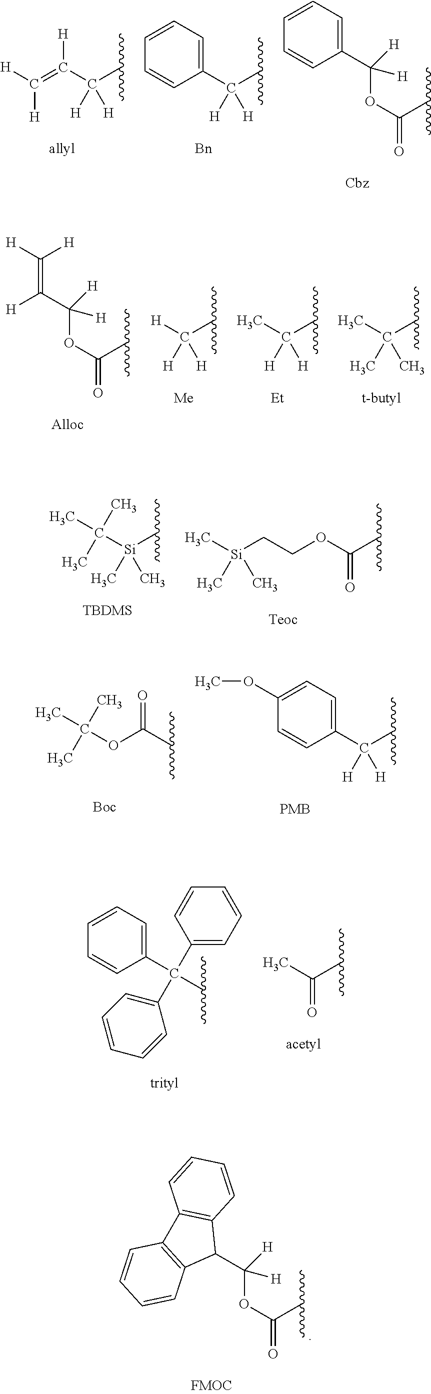

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

Certain compounds of the disclosure in Formula I or II can be conveniently prepared from the corresponding substituted 4-bromobenzenesulfonyl chloride (A-1) using general Scheme A set forth herein. In this method, the desired amine A-2 is reacted with sulfonyl chloride A-1 to produce sulfonamide A-3. Subsequent treatment with amine A-4 using a palladium catalyst like Pd₂(dba)₃, and a ligand like xantphos gives intermediate A-5. This reaction, commonly called a Buchwald-Hartwig amination, can be used with a wide variety of palladium catalyst, ligands and amines. Bromination of the central ring using a brominating reagent like bromine or NBS conveniently gives bromide A-6. Treatment of bromide A-6 with amine A-7 and a palladium catalyst like Pd(OAc)₂ and a ligand like xantphos while under a carbon monoxide atmosphere gives the desired amide A-8.

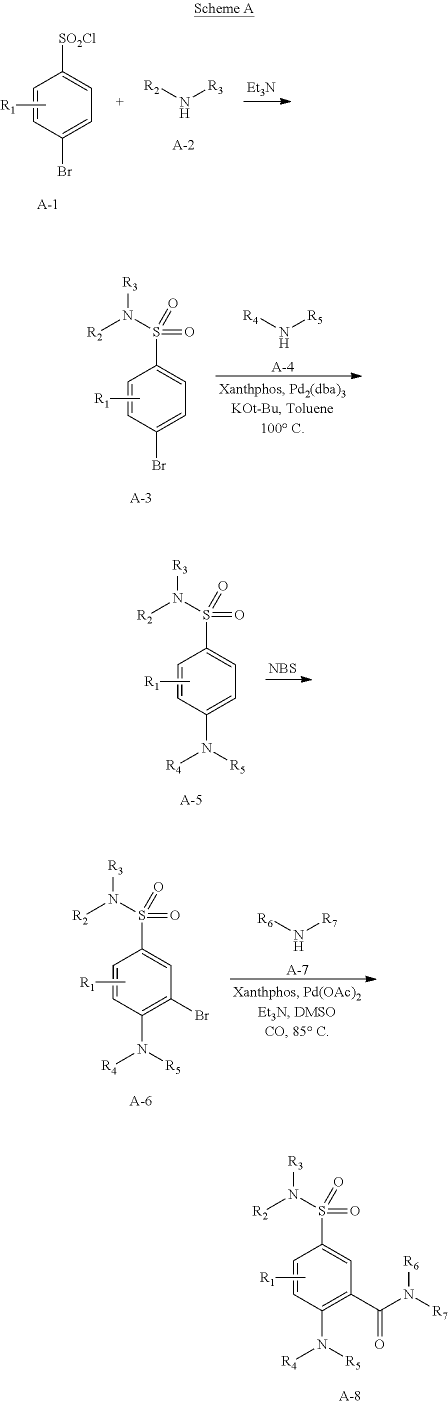

Certain examples of general formula I or II can often be made using general Method B. In this method, the desired 5-(chlorosulfonyl)-2-fluorobenzoic acid B-1 is coupled with amine B-2 to form sulfonamide B-3. Subsequent coupling with amine B-4 using standard coupling methods like EDC, CDI or HATU give the desired amide B-5. Alternatively, carboxylic acid B-3 can be converted to the corresponding acid chloride and coupled directly with amine B-4 using a simple base like triethylamine or potassium carbonate. Nucleophilic aromatic substitution of the fluoride with amine B-6 gives the target compound B-7. This reaction can be accomplished using many general methods, but simply heating in DMSO or using a microwave reactor is a non-limiting embodiment.

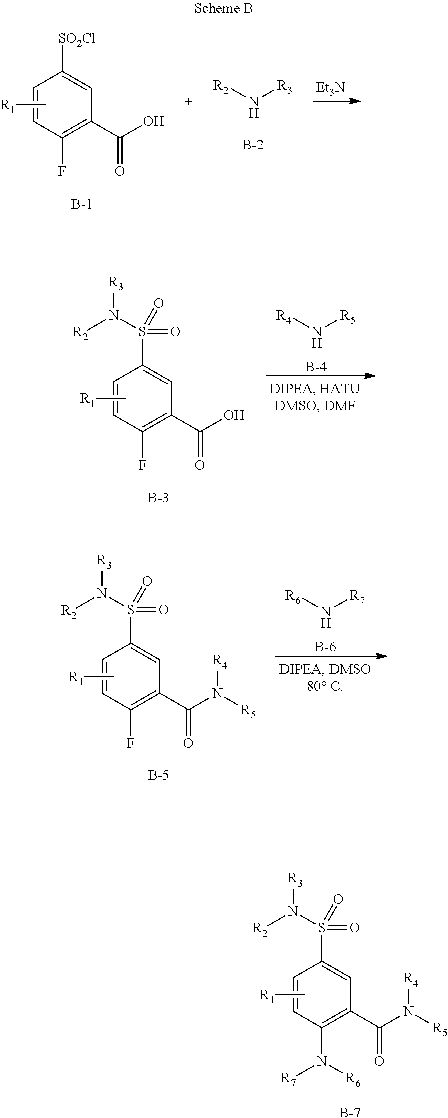

Certain examples of general formula II where Y is nitrogen can often be made using general Method C. In this method, 2-oxo-1,2-dihydropyridine-3-carboxylic acid (C-1) is treated with fuming sulfuric acid with heat to give sulfonic acid C-2. Esterification with an alcohol, non-limiting examples including methanol and ethanol, give the corresponding ester C-3. Treatment with thionyl chloride in dichloromethane with catalytic DMF gives sulfonyl chloride C-4. Coupling with amine C-5 using triethylamine in dichloromethane at 0° C. selectively gives the desired sulfonamide C-6. Subsequent coupling with amine C-7 at 60° C. gives amine C-8. Hydrolysis of the ester with aqueous sodium hydroxide followed by amide a coupling reaction with amine C-10 give target compound C-11.

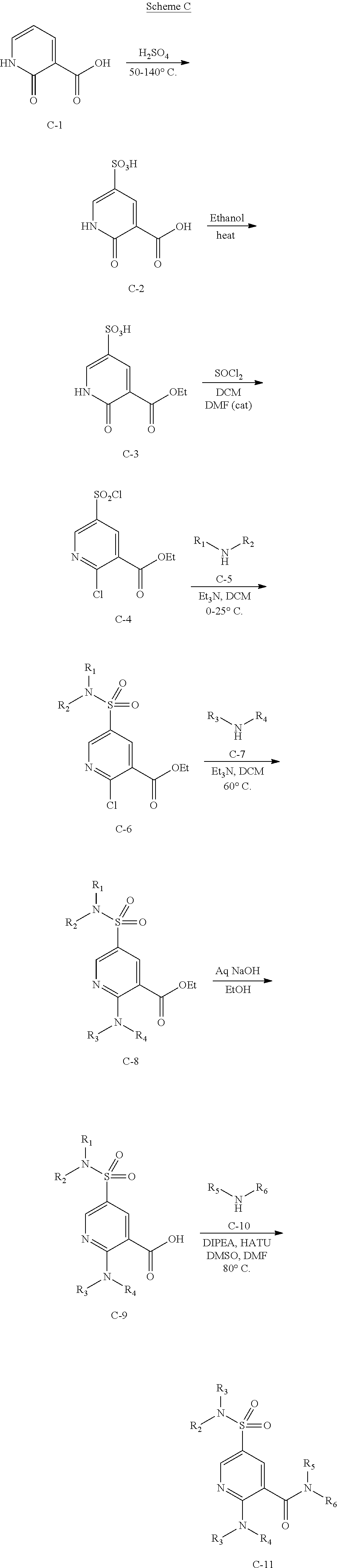

Certain examples of general formula II where Y is nitrogen can often be made using general Method D. In this method 5-bromo-6-chloropyridine-3-sulfonyl chloride (D-1) is treated with amine D-2 to give sulfonamide D-3. Nucleophilic aromatic substitution of the chloride with amine D-4 gives intermediate D-5. Carbonylative coupling with amine D-6 using xantphos, a palladium catalyst, triethylamine and carbon monoxide gives target compound D-7.

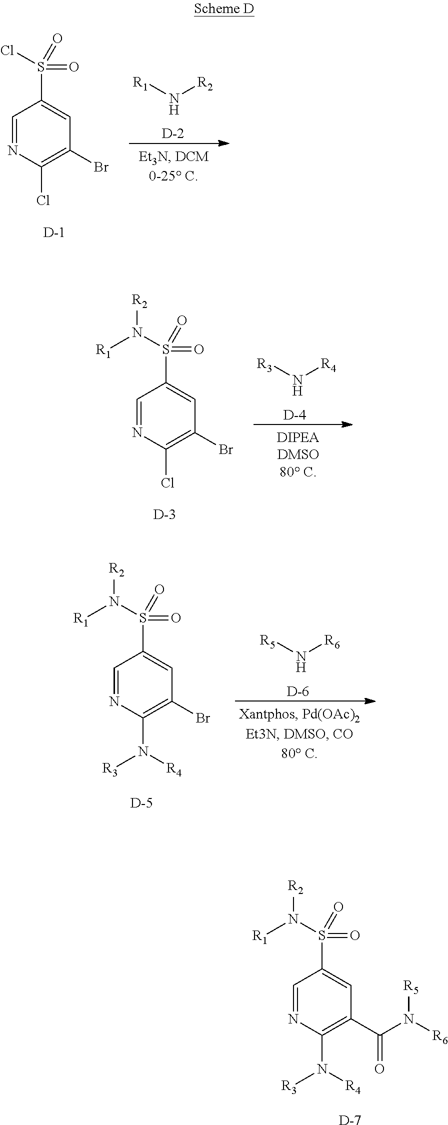

Certain examples of general formula II where X and Y are nitrogen can often be made using general Method E. In this method 3,6-dichloropyridazine-4-carboxylic acid (E-1) is esterified to give ester E-2. This can be accomplished using many known methods, but iodomethane or trimethylsilyldiazomethane are often convenient. Nucleophilic aromatic substitution with amine E-3 selectively gives intermediate E-4. Subsequent displacement of the second chloride with benzylmercaptan facilitated by use of abase like sodium hydride gives the corresponding sulfide (E-5) which is readily oxidized to the sulfonyl chloride (E-6) using NCS in aqueous acetic acid. Reaction with amine E-7 using a base like triethylamine in dichloromethane gives sulfonamide E-8. Hydrolysis of the ester gives carboxylic acid E-9, which after treatment with oxalyl chloride gives acid chloride E-10. Finally, coupling with amine E-11 gives target amide E-12.

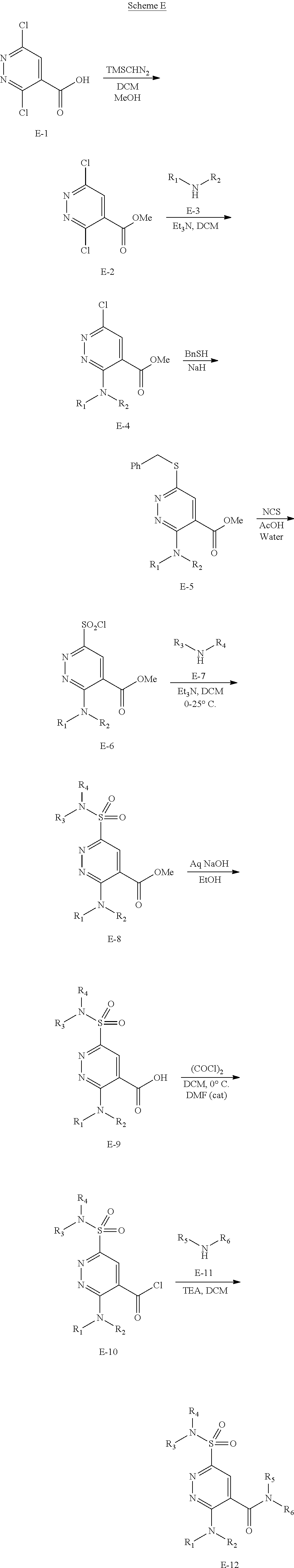

Certain examples of general formula II where the central ring is substituted with a fluorine can often be made using general method F. In this method 3,4-difluorobenzenesulfonyl chloride (F-1) is treated with amine F-2 using mild conditions to selectively give the desired sulfonamide F-3. Subsequent treatment with amine F-4 at elevated temperatures gives the nucleophilic aromatic substitution product F-5. Bromination with a brominating reagent, non-limiting examples including NBS and bromine, gives bromide F-6. Finally, carbonylative coupling with amine F-7 gives target compound F-8.

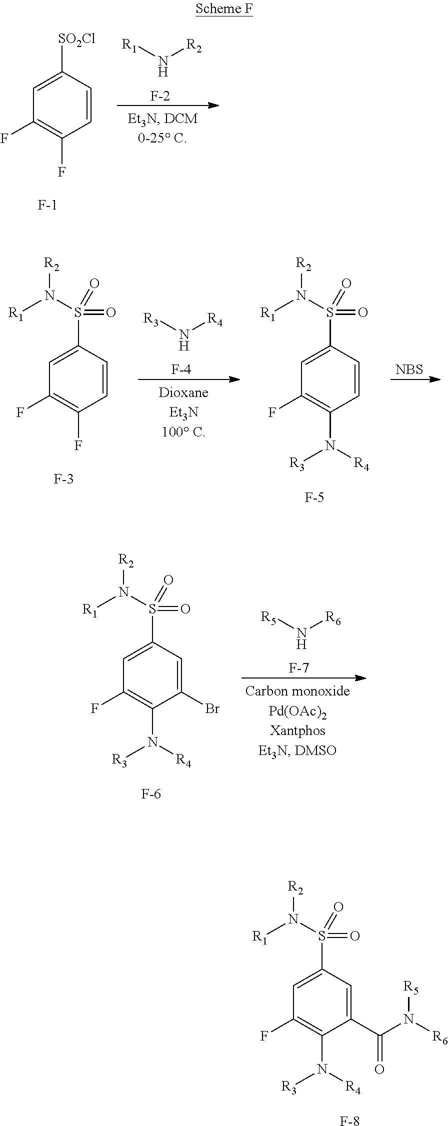

Those having skill in the art will recognize that the starting materials and reaction conditions may be varied, the sequence of the reactions altered, and additional steps employed to produce compounds encompassed by the present disclosure, as demonstrated by the following examples. In some cases, protection of certain reactive functionalities may be necessary to achieve some of the transformations contemplated herein. In general, the need for such protecting groups as well as the conditions necessary to attach and remove such groups will be apparent to those skilled in the art of organic synthesis.

The preparation of the compounds of the present disclosure is illustrated further elsewhere herein. which is not to be construed as limiting the disclosure in scope or spirit to the specific procedures and compounds described in them.

### Compositions

The disclosure includes a pharmaceutical composition comprising at least one compound of the disclosure and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

### Methods

The disclosure includes a method of treating or preventing a disease associated with N-linked glycosylation in a subject in need thereof. In certain embodiments, the disease comprises a cancer.

As demonstrated herein, the compounds of the present disclosure inhibit N-linked glycosylation. In certain embodiments, such inhibition comprises oligosaccharyltransferase (OST) inhibition. In other embodiments, the compounds of the present disclosure inhibit growth and/or kill cancer cells that are dependent on receptor tyrosine kinases (RTKs) for proliferation. Thus, provided herein is a method of inhibiting or disrupting N-linked glycosylation in a cell, which includes contacting the cell with an effective amount of at least one agent selected from the group consisting of formula (I), formula (II), and formula (III) and/or a pharmaceutical composition of the present disclosure.

The agent can inhibit or disrupt at least one cell process selected from the group consisting of oligosaccharyltransferase function and receptor tyrosine kinase cell surface expression.

In various embodiments, the cell is a receptor tyrosine kinase-dependent cancer cell. The agent can cause, in some embodiments, at least one cellular effect selected from the group consisting of cell cycle arrest and/or senescence, and cell proliferation blockage or inhibition. The cell can be a mammalian cell, including a human cell. The agent can, in various embodiments, be administered to the mammal by any route of administration described herein.

In certain embodiments, a method of preventing or treating a cancer in a subject in need thereof is provided herein and includes, administering to the subject a therapeutically effective amount of at least one agent selected from the group consisting of formula (I), formula (II), and formula (III) or a pharmaceutical composition of the present disclosure. The agent can block or inhibit cell surface expression of the receptor tyrosine kinase in a cell from the cancer.

In some embodiments the method includes administering to the subject at least one other therapeutic compound. The agent and the at least one additional therapeutic compound can be co-administered to the subject. In some embodiments, the agent and the at least one additional therapeutic compound are coformulated.

The methods of the disclosure include administering to the subject a therapeutically effective amount of at least one compound of the disclosure, which is optionally formulated in a pharmaceutical composition. In certain embodiments, the method further includes administering to the subject an additional therapeutic agent that treats or prevents cancer.

Agents of formula (I), formula (II), or formula (III) can be administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.

Examples of cancers that can be treated or prevented by the present disclosure include but are not limited to: squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer comprises small cell lung cancer, non-small cell lung cancer, head and neck squamous cell carcinoma, breast cancer, gastric cancer, cervical cancer, colon cancer, and glioma.

In certain embodiments, administering the compound and/or pharmaceutical composition of the disclosure to the subject allows for administering a lower dose of the additional therapeutic agent as compared to the dose of the additional therapeutic agent alone that is required to achieve similar results in treating or preventing a cancer in the subject. For example, in certain embodiments, the compound and/or composition of the disclosure enhances the anti-cancer activity of the additional therapeutic compound, thereby allowing for a lower dose of the additional therapeutic compound to provide the same effect.

In certain embodiments, the compound of the disclosure and the therapeutic agent are co-administered to the subject. In other embodiments, the compound of the disclosure and the therapeutic agent are coformulated and co-administered to the subject.

In certain embodiments, the subject is a mammal. In other embodiments, the mammal is a human.

### Combination Therapies

The compounds useful within the methods of the disclosure may be used in combination with one or more additional therapeutic agents useful for treating a cancer. These additional therapeutic agents may comprise compounds that are commercially available or synthetically accessible to those skilled in the art. These additional therapeutic agents are known to treat, prevent, or reduce the symptoms, of a cancer.

In non-limiting examples, the compounds useful within the disclosure may be used in combination with one or more of the following therapeutic agents: Erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), pemetrexed (ALIMTA®, Eli Lilly), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, rapamycin, oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), trametinib (Mek inhibitor), selumetinib (Mek inhibitor), vemurafenib (B-raf inhibitor), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA®, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®), ALK TKI inhibitors, antibodies such as avastin and cetuximab that target VEGFR and EGFR respectively, other RTK TKIs for PDGFR or RET, immunotherapies such as ipiliumimab (targeting CTLA-4), and nivolumab (targeting PD-1), pembrolizumab (targeting PD-1), cemiplimab (targeting PD-1), atezolizumab (targeting PD-L1), avelumab (targeting PD-L1), durvalumab (targeting PD-L1), and radiation therapy.

In certain embodiments, the compounds of the present disclosure are used in combination with radiation therapy. In other embodiments, the combination of administration of the compounds of the present disclosure and application of radiation therapy is more effective in treating or preventing cancer than application of radiation therapy by itself. In yet other embodiments, the combination of administration of the compounds of the present disclosure and application of radiation therapy allows for use of lower amount of radiation therapy in treating the subject.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to herein may be applied to experimental data to generate a corresponding graph to aid in assessing the effects of the drug combination. The corresponding graphs associated with the equations referred to herein are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, preferably a mammal, more preferably a human, may be carried out using known procedures, at dosages and for periods of time effective to treat a cancer in the patient. An effective amount of the therapeutic compound necessary to achieve a therapeutic effect may vary according to factors such as the state of the disease or disorder in the patient; the age, sex, and weight of the patient; and the ability of the therapeutic compound to treat a cancer in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a cancer in a patient.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a cancer in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present disclosure also includes a multi-layer tablet comprising a layer providing for the delayed release of one or more compounds of the disclosure, and a further layer providing for the immediate release of a medication for treatment of G-protein receptor-related diseases or disorders. Using a wax/pH-sensitive polymer mix, a gastric insoluble composition may be obtained in which the active ingredient is entrapped, ensuring its delayed release.

Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In one embodiment of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present disclosure depends on the age, sex and weight of the patient, the current medical condition of the patient and the progression of a cancer in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

### Methods and Materials

### Cell Lines and Culture Conditions

The CHO-ER-LucT cells have been previously described (Contessa et al., 2010, Clinical Cancer Research, 16(12):3205-14) and are used for testing analog IC₅₀ for inhibition of N-linked glycosylation in 96 or 384 well plate format. In these cells induction of luminescence is dependent on inhibition of glycosylation (and OST inhibition), and is determined after 24 hrs of exposure to the analog following the addition of luciferin substrate. The luminescent signal is then quantified using standard plate readers that detect luminescence (e.g., Biotek synergy). Experiments are performed over a seven-point dose dilution to generate dose response data and to identify IC₅₀ values and maximal activity compared to controls. Data is transformed to log 10 values to generate a sigmoidal shaped dose response curve and fitted using nonlinear regression (curve fit) in GraphPad Prism7. IC₅₀ values for OST inhibitors are reported in Table 2.

Inhibition of glycosylation is then confirmed by analysis of glycoprotein size using western blots. Increased gel mobility of proteins such as the EGFR or Halo3N indicate loss of protein glycosylation (FIGS. 1-2).

### Chemical Synthesis of NGI-1

Comparator compound NGI-1 can be prepared using the general procedure shown herein:

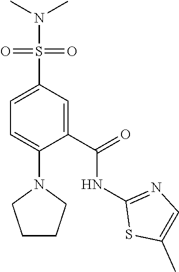 [SMILES: Cc1cnc(NC(=O)c2cc(S(=O)(=O)N(C)C)ccc2N2CCCC2)s1]

### Methyl 5-(chlorosulfonyl)-2-hydroxybenzoate

To a round-bottom flask was added the chlorosulfonic acid (30 mL, 450 mmol). The flask was cooled to 0° C. and the methyl 2-hydroxybenzoate (6.4 mL, 50.0 mmol) was added dropwise over 30 min. The reaction was allowed to warm to rt and was then heated to 40° C. After 2 h the reaction was cooled to rt and poured into ice water (200 mL). The solid precipitate was filtered to produce methyl 5-(chlorosulfonyl)-2-hydroxybenzoate (11.2 g, 44.7 mmol, 90% yield) as a white solid. ¹H NMR (400 MHz, CDCl₃) δ 11.55 (s, 1H), 8.57 (d, J=2.6 Hz, 1H), 8.09 (dd, J=9.0, 2.6 Hz, 1H), 7.18 (d, J=9.0 Hz, 1H), 4.04 (s, 3H).

### Methyl 5-(N,N-dimethylsulfamoyl)-2-hydroxybenzoate

To a vial was added dimethylamine hydrochloride (1.46 g, 18.0 mmol) and DIPEA (4.2 mL, 23.9 mmol) in acetonitrile (4 mL) and the reaction stirred at rt for 30 min. Methyl 5-(chlorosulfonyl)-2-hydroxybenzoate (1.50 g, 6.0 mmol) was then added portion-wise over 10 minutes and after addition the reaction stirred at rt for 2 h. At this time the reaction was concentrated and the residue was diluted with saturated NaHCO₃ (20 mL) and extracted with EtOAc (3×20 mL). The organic layers were combined and dried over MgSO₄, filtered and concentrated. The crude product was dissolved in 50% EtOAc:hexanes (20 mL) and filtered through a silica plug. The eluent was collected and concentrated to provide methyl 5-(N,N-dimethylsulfamoyl)-2-hydroxybenzoate (1.36 g, 5.25 mmol, 88% yield) as a white solid. ¹H NMR (400 MHz, CDCl₃) δ 11.24 (s, 1H), 8.30 (d, J=2.3 Hz, 1H), 7.84 (dd, J=8.8, 2.4 Hz, 1H), 7.12 (d, J=8.8 Hz, 1H), 4.00 (s, 3H), 2.71 (s, 6H).

### Methyl 5-(N,N-dimethylsulfamoyl)-2-(((trifluoromethyl)sulfonyl)oxy)benzoate

To a vial was added methyl 5-(N,N-dimethylsulfamoyl)-2-hydroxybenzoate (0.36 g, 1.40 mmol) and CH₂Cl₂ (4 mL) followed by pyridine (0.23 mL, 2.79 mmol). The reaction was then cooled to 0° C. and the 1.0 M solution of trifluoromethanesulfonic anhydride in CH₂Cl₂ (1.68 mL, 1.68 mmol) was added dropwise over 10 minutes. The reaction was then allowed to warm to rt and stirred for 3 h at which point it was washed with saturated NaHCO₃ (10 mL) and the CH₂Cl₂ was extracted. The remaining aqueous layer was extracted further with CH₂Cl₂ (2×10 mL). The organic layers were combined and dried over MgSO₄, filtered and concentrated to produce methyl 5-(N,N-dimethylsulfamoyl)-2-(((trifluoromethyl)sulfonyl)oxy)benzoate (0.50 g, 1.28 mmol, 92% yield) as a white solid. ¹H NMR (400 MHz, CDCl₃) δ 8.47 (d, J=2.4 Hz, 1H), 8.03 (dd, J=8.6, 2.4 Hz, 1H), 7.49 (d, J=8.6 Hz, 1H), 4.01 (s, 3H), 2.80 (s, 6H).

### Methyl 5-(N,N-dimethylsulfamoyl)-2-(pyrrolidin-1-yl)benzoate

To a vial was added methyl 5-(N,N-dimethylsulfamoyl)-2-(((trifluoromethyl) sulfonyl)oxy)benzoate (0.96 g, 2.46 mmol), pyrrolidine (0.61 mL, 7.38 mmol) and acetonitrile. The reaction was heated to 80° C. and stirred for 1 h, at which point the reaction was removed from heat and allowed to cool to rt. The reaction was then concentrated and the residue was re-dissolved in EtOAc (15 mL) and washed with saturated NaHCO₃ (15 mL), dried over MgSO₄, filtered and concentrated to produce methyl 5-(N,N-dimethylsulfamoyl)-2-(pyrrolidin-1-yl)benzoate (0.73 g, 2.34 mmol, 95% yield) as a light yellow solid. ¹H NMR (400 MHz, CDCl₃) δ 7.97 (d, J=2.4 Hz, 1H), 7.64 (dd, J=9.0, 2.4 Hz, 1H), 6.80 (d, J=9.0 Hz, 1H), 3.90 (s, 3H), 3.34-3.25 (m, 4H), 2.68 (s, 6H), 2.03-1.97 (m, 4H).

### 5-(N,N-Dimethylsulfamoyl)-2-(pyrrolidin-1-yl)benzoic acid

To a vial was added methyl 5-(N,N-dimethylsulfamoyl)-2-(pyrrolidin-1-yl)benzoate (0.73 g, 2.34 mmol), MeOH (4 mL) and 5.0 M NaOH in water (4.69 mL, 23.4 mmol) and the reaction stirred at 90° C. for 1.5 h. The reaction was then allowed to cool to rt and the methanol was evaporated in vacuo. The remaining aqueous layer was acidified with 6.0 M HCl to pH 2-3 and extracted with CH₂Cl₂ (4×10 mL). The organic layers were combined and dried over MgSO₄, filtered and concentrated to produce 5-(N,N-dimethylsulfamoyl)-2-(pyrrolidin-1-yl)benzoic acid (0.61 g, 2.05 mmol, 87% yield) as a white solid. ¹H NMR (400 MHz, CDCl₃) δ 8.38 (d, J=2.4 Hz, 1H), 7.82 (dd, J=8.8, 2.2 Hz, 1H), 7.17 (d, J=8.7 Hz, 1H), 3.37-3.27 (m, 4H), 2.73 (d, J=0.5 Hz, 6H), 2.13-2.03 (m, 4H).

### 5-(N,N-Dimethylsulfamoyl)-N-(5-methylthiazol-2-yl)-2-(pyrrolidin-1-yl)benzamide NGI-1

To a vial was added 5-(N,N-dimethylsulfamoyl)-2-(pyrrolidin-1-yl)benzoic acid (0.20 g, 0.66 mmol), HBTU (0.37 g, 0.99 mmol), DIPEA (0.29 mL, 1.65 mmol) and DMF (1.0 mL) and the reaction stirred at RT for 15 minutes. Then 5-methyl thiazol-2-amine (0.11 g, 0.99 mmol) was added to the reaction and it was heated to 100° C. and stirred for 3.5 h. The this time the reaction was allowed to cool to rt and diluted with water (15 mL) then extracted with CH₂Cl₂ (2×15 mL). The organic layers were combined and washed with water (4×30 mL), dried over MgSO₄, filtered and adsorbed to Celite then purified by reverse-phase flash chromatography (10-100% MeCN:water). The fractions containing the product were then concentrated and adsorbed to silica and purified by flash chromatography (0-5% MeOH:CH₂Cl₂) to produce 5-(N,N-dimethylsulfamoyl)-N-(5-methylthiazol-2-yl)-2-(pyrrolidin-1-yl)benzamide (0.091 g, 0.23 mmol, 35% yield). ¹H NMR (400 MHz, CDCl₃) δ 12.77 (s, 1H), 7.95 (d, J=2.3 Hz, 1H), 7.73 (dd, J=9.0, 2.3 Hz, 1H), 6.89 (d, J=9.0 Hz, 1H), 6.77 (s, 1H), 3.40-3.28 (m, 4H), 2.71 (s, 6H), 2.40 (d, J=1.3 Hz, 3H), 2.05-1.95 (m, 4H). ¹³C NMR (125 MHz, CDCl₃) δ 166.30, 157.62, 149.11, 133.89, 130.95, 130.40, 127.40, 121.27, 119.30, 114.16, 50.53, 38.00, 25.75, 11.55. LCMS-UV Purity at 214 nm: 100%. HRMS (ESI): m/z calcd for C₁₇H₂₂N₄O₃S₂ (M+H⁺) 395.1211, found 395.1211.

TABLE 1
Selected Examples

| Example | | |
|---|---|---|
| No. | Structure | Name |
| 1 | 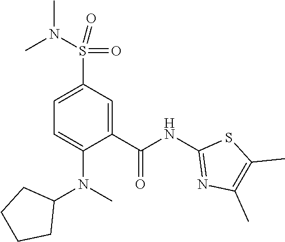 [SMILES: Cc1nc(NC(=O)c2cc(S(=O)(=O)N(C)C)ccc2N(C)C2CCCC2)sc1C] | 2-(cyclopentyl(methyl)amino)-5-(N,N- dimethylsulfamoyl)-N-(4,5-dimethylthiazol- 2-yl)benzamide |
| 2 | 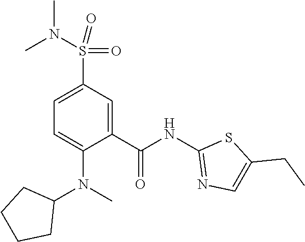 [SMILES: CCc1cnc(NC(=O)c2cc(S(=O)(=O)N(C)C)ccc2N(C)C2CCCC2)s1] | 2-(cyclopentyl(methyl)amino)-5-(N,N- dimethylsulfamoyl)-N-(5-ethylthiazol-2- yl)benzamide |
| 3 | 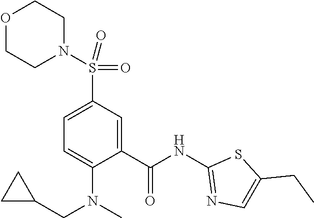 [SMILES: CCc1cnc(NC(=O)c2cc(S(=O)(=O)N3CCOCC3)ccc2N(C)CC2CC2)s1] | 2-((cyclopropylmethyl)(methyl)amino)-N-(5- ethylthiazol-2-yl)-5- (morpholinosulfonyl)benzamide |
| 4 | 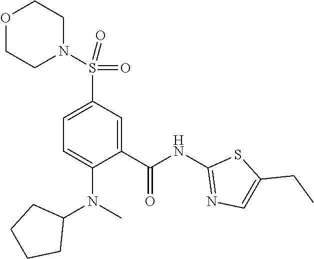 [SMILES: CCc1cnc(NC(=O)c2cc(S(=O)(=O)N3CCOCC3)ccc2N(C)C2CCCC2)s1] | 2-(cyclopentyl(methyl)amino)-N-(5- ethylthiazol-2-yl)-5- (morpholinosulfonyl)benzamide |
| 5 | 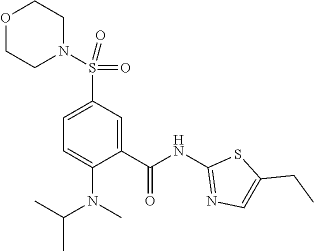 [SMILES: CCc1cnc(NC(=O)c2cc(S(=O)(=O)N3CCOCC3)ccc2N(C)C(C)C)s1] | N-(5-ethylthiazol-2-yl)-2- (isopropyl(methyl)amino)-5- (morpholinosulfonyl)benzamide |
| 6 | 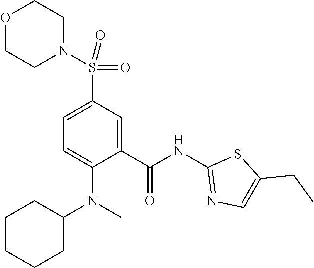 [SMILES: CCc1cnc(NC(=O)c2cc(S(=O)(=O)N3CCOCC3)ccc2N(C)C2CCCCC2)s1] | 2-(cyclohexyl(methyl)amino)-N-(5- ethylthiazol-2-yl)-5- (morpholinosulfonyl)benzamide |
| 7 | 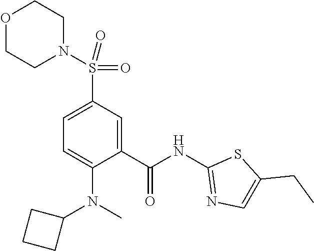 [SMILES: CCc1cnc(NC(=O)c2cc(S(=O)(=O)N3CCOCC3)ccc2N(C)C2CCC2)s1] | 2-(cyclobutyl(methyl)amino)-N-(5- ethylthiazol-2-yl)-5- (morpholinosulfonyl)benzamide |
| 8 | 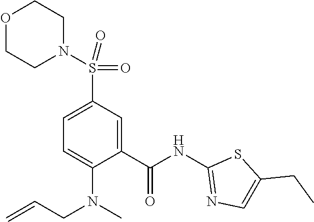 [SMILES: C=CCN(C)c1ccc(S(=O)(=O)N2CCOCC2)cc1C(=O)Nc1ncc(CC)s1] | 2-(allyl(methyl)amino)-N-(5-ethylthiazol-2- yl)-5-(morpholinosulfonyl)benzamide |
| 9 | 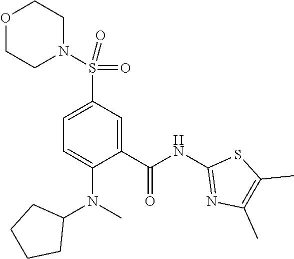 [SMILES: Cc1nc(NC(=O)c2cc(S(=O)(=O)N3CCOCC3)ccc2N(C)C2CCCC2)sc1C] | 2-(cyclopentyl(methyl)amino)-N-(4,5- dimethylthiazol-2-yl)-5- (morpholinosulfonyl)benzamide |
| 10 | 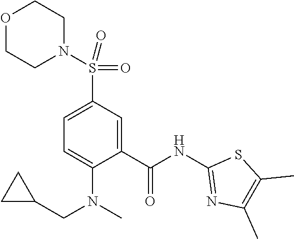 [SMILES: Cc1nc(NC(=O)c2cc(S(=O)(=O)N3CCOCC3)ccc2N(C)CC2CC2)sc1C] | 2-((cyclopropylmethyl)(methyl)amino)-N- (4,5-dimethylthiazol-2-yl)-5- (morpholinosulfonyl)benzamide |
| 11 | 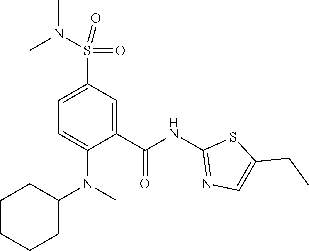 [SMILES: CCc1cnc(NC(=O)c2cc(S(=O)(=O)N(C)C)ccc2N(C)C2CCCCC2)s1] | 2-(cyclohexyl(methyl)amino)-5-(N,N- dimethylsulfamoyl)-N-(5-ethylthiazol-2- yl)benzamide |
| 12 | 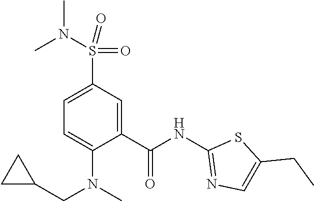 [SMILES: CCc1cnc(NC(=O)c2cc(S(=O)(=O)N(C)C)ccc2N(C)CC2CC2)s1] | 2-((cyclopropylmethyl)(methyl)amino)-5- (N,N-dimethylsulfamoyl)-N-(5-ethylthiazol- 2-yl)benzamide |
| 13 | 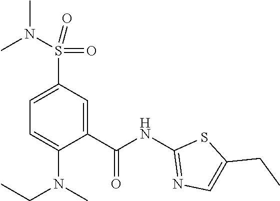 [SMILES: CCc1cnc(NC(=O)c2cc(S(=O)(=O)N(C)C)ccc2N(C)CC)s1] | 5-(N,N-dimethylsulfamoyl)-2- (ethyl(methyl)amino)-N-(5-ethylthiazol-2- yl)benzamide |
| 14 | 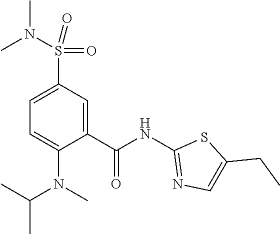 [SMILES: CCc1cnc(NC(=O)c2cc(S(=O)(=O)N(C)C)ccc2N(C)C(C)C)s1] | 5-(N,N-dimethylsulfamoyl)-N-(5- ethylthiazol-2-yl)-2- (isopropyl(methyl)amino)benzamide |
| 15 | 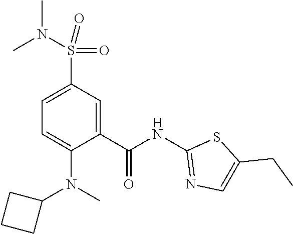 [SMILES: CCc1cnc(NC(=O)c2cc(S(=O)(=O)N(C)C)ccc2N(C)C2CCC2)s1] | 2-(cyclobutyl(methyl)amino)-5-(N,N- dimethylsulfamoyl)-N-(5-ethylthiazol-2- yl)benzamide |
| 16 | 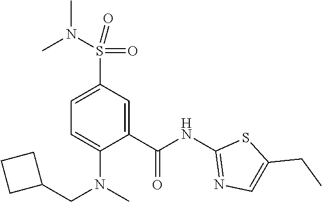 [SMILES: CCc1cnc(NC(=O)c2cc(S(=O)(=O)N(C)C)ccc2N(C)CC2CCC2)s1] | 2-((cyclobutylmethyl)(methyl)amino)-5- (N,N-dimethylsulfamoyl)-N-(5-ethylthiazol- 2-yl)benzamide |
| 17 | 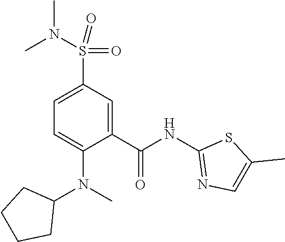 [SMILES: Cc1cnc(NC(=O)c2cc(S(=O)(=O)N(C)C)ccc2N(C)C2CCCC2)s1] | 2-(cyclopentyl(methyl)amino)-5-(N,N- dimethylsulfamoyl)-N-(5-methylthiazol-2- yl)benzamide |
| 18 | 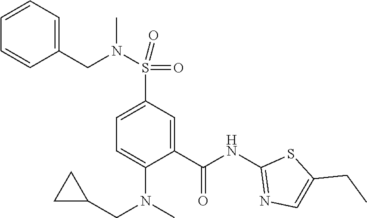 [SMILES: CCc1cnc(NC(=O)c2cc(S(=O)(=O)N(C)Cc3ccccc3)ccc2N(C)CC2CC2)s1] | 5-(N-benzyl-N-methylsulfamoyl)-2- ((cyclopropylmethyl)(methyl)amino)-N-(5- ethylthiazol-2-yl)benzamide |
| 19 | 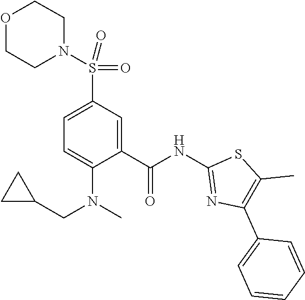 [SMILES: Cc1sc(NC(=O)c2cc(S(=O)(=O)N3CCOCC3)ccc2N(C)CC2CC2)nc1-c1ccccc1] | 2-((cyclopropylmethyl)(methyl)amino)-N-(5- methyl-4-phenylthiazol-2-yl)-5- (morpholinosulfonyl)benzamide |
| 20 | 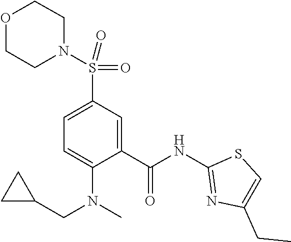 [SMILES: CCc1csc(NC(=O)c2cc(S(=O)(=O)N3CCOCC3)ccc2N(C)CC2CC2)n1] | 2-((cyclopropylmethyl)(methyl)amino)-N-(4- ethylthiazol-2-yl)-5- (morpholinosulfonyl)benzamide |
| 21 | 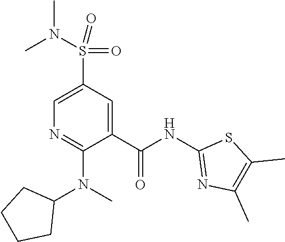 [SMILES: Cc1nc(NC(=O)c2cc(S(=O)(=O)N(C)C)cnc2N(C)C2CCCC2)sc1C] | 2-(cyclopentyl(methyl)amino)-5-(N,N- dimethylsulfamoyl)-N-(4,5-dimethylthiazol- 2-yl)nicotinamide |
| 22 | 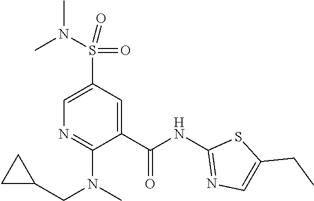 [SMILES: CCc1cnc(NC(=O)c2cc(S(=O)(=O)N(C)C)cnc2N(C)CC2CC2)s1] | 2-((cyclopropylmethyl)(methyl)amino)-5- (N,N-dimethylsulfamoyl)-N-(5-ethylthiazol- 2-yl)nicotinamide |
| 23 | 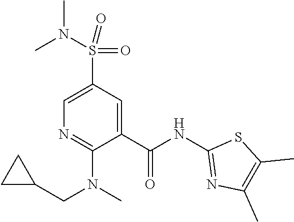 [SMILES: Cc1nc(NC(=O)c2cc(S(=O)(=O)N(C)C)cnc2N(C)CC2CC2)sc1C] | 2-((cyclopropylmethyl)(methyl)amino)-5- (N,N-dimethylsulfamoyl)-N-(4,5- dimethylthiazol-2-yl)nicotinamide |
| 24 | 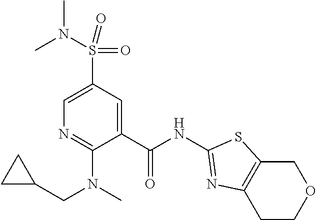 [SMILES: CN(CC1CC1)c1ncc(S(=O)(=O)N(C)C)cc1C(=O)Nc1nc2c(s1)COCC2] | 2-((cyclopropylmethyl)(methyl)amino)-N- (6,7-dihydro-4H-pyrano[4,3-d]thiazol-2-yl)-5- (N,N-dimethylsulfamoyl)nicotinamide |
| 25 | 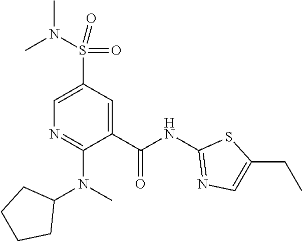 [SMILES: CCc1cnc(NC(=O)c2cc(S(=O)(=O)N(C)C)cnc2N(C)C2CCCC2)s1] | 2-(cyclopentyl(methyl)amino)-5-(N,N- dimethylsulfamoyl)-N-(5-ethylthiazol-2- yl)nicotinamide |
| 26 | 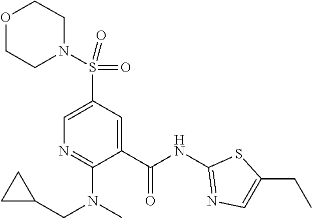 [SMILES: CCc1cnc(NC(=O)c2cc(S(=O)(=O)N3CCOCC3)cnc2N(C)CC2CC2)s1] | 2-((cyclopropylmethyl)(methyl)amino)-N-(5- ethylthiazol-2-yl)-5- (morpholinosulfonyl)nicotinamide |
| 27 | 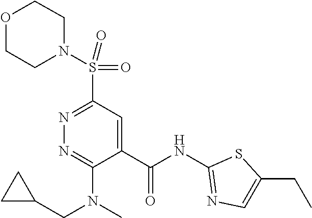 [SMILES: CCc1cnc(NC(=O)c2cc(S(=O)(=O)N3CCOCC3)nnc2N(C)CC2CC2)s1] | 3-((cyclopropylmethyl)(methyl)amino)-N-(5- ethylthiazol-2-yl)-6- (morpholinosulfonyl)pyridazine-4- carboxamide |
| 28 | 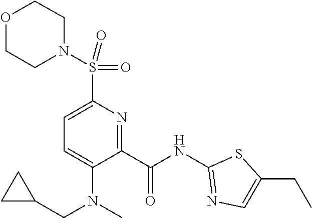 [SMILES: CCc1cnc(NC(=O)c2nc(S(=O)(=O)N3CCOCC3)ccc2N(C)CC2CC2)s1] | 3-((cyclopropylmethyl)(methyl)amino)-N-(5- ethylthiazol-2-yl)-6- (morpholinosulfonyl)picolinamide |
| 29 | 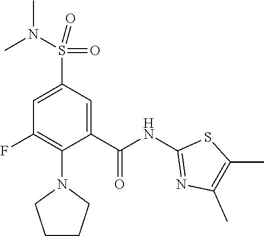 | 5-(N,N-dimethylsulfamoyl)-N-(4,5- dimethylthiazol-2-yl)-3-fluoro-2-(pyrrolidin- 1-yl)benzamide |
| 30 | 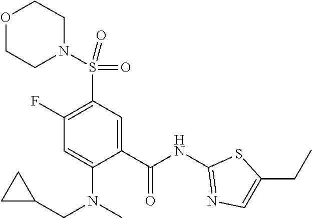 [SMILES: CCc1cnc(NC(=O)c2cc(S(=O)(=O)N3CCOCC3)c(F)cc2N(C)CC2CC2)s1] | 2-((cyclopropylmethyl)(methyl)amino)-N-(5- ethylthiazol-2-yl)-4-fluoro-5- (morpholinosulfonyl)benzamide |
| 31 | 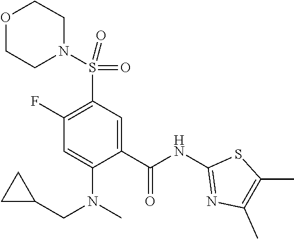 [SMILES: Cc1nc(NC(=O)c2cc(S(=O)(=O)N3CCOCC3)c(F)cc2N(C)CC2CC2)sc1C] | 2-((cyclopropylmethyl)(methyl)amino)-N- (4,5-dimethylthiazol-2-yl)-4-fluoro-5- (morpholinosulfonyl)benzamide |
| 32 | 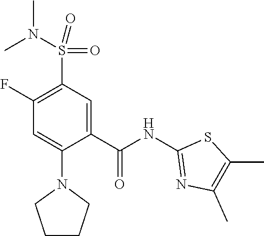 | 5-(N,N-dimethylsulfamoyl)-N-(4,5- dimethylthiazol-2-yl)-4-fluoro-2-(pyrrolidin- 1-yl)benzamide |
| 33 | 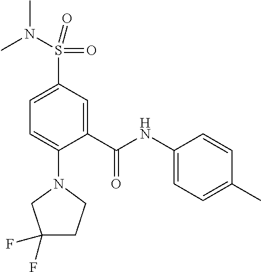 | 2-(3,3-difluoropyrrolidin-1-yl)-5-(N,N- dimethylsulfamoyl)-N-(p-tolyl)benzamide |
| 34 | 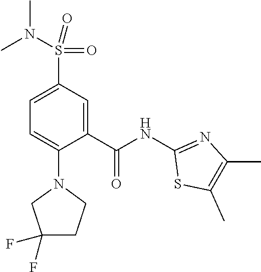 | 2-(3,3-difluoropyrrolidin-1-yl)-5-(N,N- dimethylsulfamoyl)-N-(4,5-dimethylthiazol- 2-yl)benzamide |
| 35 | 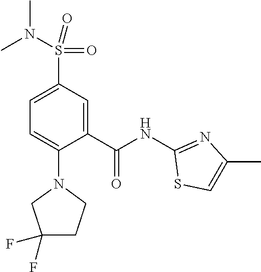 | 2-(3,3-difluoropyrrolidin-1-yl)-5-(N,N- dimethylsulfamoyl)-N-(4-methylthiazol-2- yl)benzamide |
| 36 | 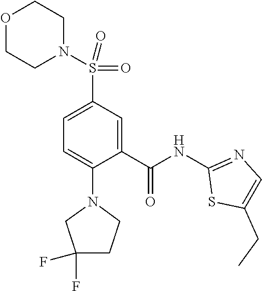 [SMILES: CCc1cnc(NC(=O)c2cc(S(=O)(=O)N3CCOCC3)ccc2N2CCC(F)(F)C2)s1] | 2-(3,3-difluoropyrrolidin-1-yl)-N-(5- ethylthiazol-2-yl)-5- (morpholinosulfonyl)benzamide |
| 37 | 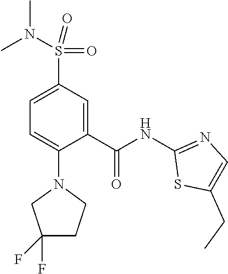 [SMILES: CCc1cnc(NC(=O)c2cc(S(=O)(=O)N(C)C)ccc2N2CCC(F)(F)C2)s1] | 2-(3,3-difluoropyrrolidin-1-yl)-5-(N,N- dimethylsulfamoyl)-N-(5-ethylthiazol-2- yl)benzamide |
| 38 | 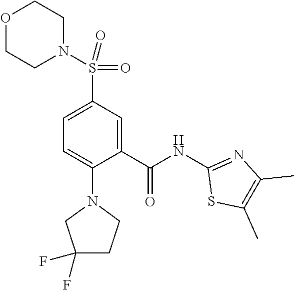 [SMILES: Cc1nc(NC(=O)c2cc(S(=O)(=O)N3CCOCC3)ccc2N2CCC(F)(F)C2)sc1C] | 2-(3,3-difluoropyrrolidin-1-yl)-N-(4,5- dimethylthiazol-2-yl)-5- (morpholinosulfonyl)benzamide |

### Example 1: Preparation of 2-(cyclopentyl(methyl)amino)-5-(N,N-dimethylsulfamoyl)-N-(2-ethylthiazol-5-yl)benzamide

### Step 1: Synthesis of 4-bromo-N,N-dimethylbenzenesulfonamide

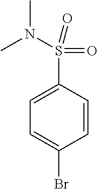

While under nitrogen, a solution of dimethylamine hydrochloride (1.72 g. 21.1 mmol) and 4-bromobenzenesulfonyl chloride (5.14 g, 20.1 mmol) in dichloromethane was cooled to 0° C. and treated with triethylamine (6.29 mL, 45.3 mmol). After stirring for 2 h at room temperature, the reaction was concentrated, diluted with ethyl acetate, and washed with water. The resulting solution was dried over anhydrous magnesium sulfate, filtered and concentrated to give the title compound as a white solid 5.25 g, 99% yield. ¹H NMR (400 MHz, Chloroform-d) δ 7.71-7.58 (m, 4H), 2.70 (s, 6H).

### Step 2: Synthesis of 4-(cyclopentylamino)-N,N-dimethylbenzenesulfonamide

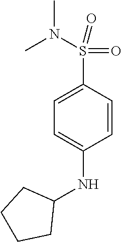 [SMILES: CN(C)S(=O)(=O)c1ccc(NC2CCCC2)cc1]

While under nitrogen, a mixture of 4-bromo-N,N-dimethylbenzene-1-sulfonamide (1.32 g, 5 mmol), (tert-butoxy)potassium (673 mg, 6 mmol), xantphos (116 mg, 0.2 mmol) and tris(dibenzylideneacetone)dipalladium(0) (92 mg, 0.1 mmol) was treated with cyclopentanamine (0.54 mL, 5.5 mmol) in toluene (5 mL) and heated to 110° C. for 12 h. After cooling to room temperature, the reaction mixture was diluted with ethyl acetate, washed with water, dried over anhydrous magnesium sulfate, filtered, and concentrated. Purification by flash column chromatography (20% ethyl acetate in hexanes) gives the title compound as a white solid (1.3 g, 97% yield). ¹H NMR (400 MHz, Chloroform-d) δ 7.61-7.45 (m, 2H), 6.60 (d, J=8.1 Hz, 2H), 3.88-3.69 (m, 1H), 2.70-2.54 (m, 6H), 2.10-1.94 (m, 2H), 1.83-1.37 (m, 6H).

### Step 3: Synthesis of 3-bromo-4-(cyclopentylamino)-N,N-dimethylbenzenesulfonamide

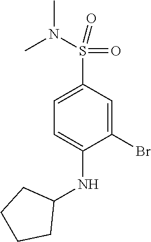 [SMILES: CN(C)S(=O)(=O)c1ccc(NC2CCCC2)c(Br)c1]

While under nitrogen, a solution of 4-(cyclopentylamino)-N,N-dimethylbenzene-1-sulfonamide (1.04 g, 3.88 mmol) in DMF (3 mL) was treated with N-bromosuccinimide (690 mg, 3.88 mmol). After stirred at 25° C. for 24 h, the reaction was concentrated, diluted with ethyl acetate, and washed with saturated aqueous NaHCO₃. The organic layer was dried over anhydrous magnesium sulfate, filtered and concentrated to give the title compound as a white solid (1.3 g, 97% yield). ¹H NMR (400 MHz, Chloroform-d) δ 7.79 (d, J=2.1 Hz, 1H), 7.54 (dd, J=8.7, 2.1 Hz, 1H), 6.66 (d, J=8.7 Hz, 1H), 4.78 (s, 1H), 3.83 (q, J=6.1 Hz, 1H), 2.66 (s, 6H), 2.13-1.99 (m, 2H), 1.79-1.51 (m, 6H).

### Step 4: Synthesis of 3-bromo-4-(cyclopentyl(methyl)amino)-N,N-dimethylbenzene sulfonamide

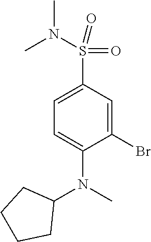 [SMILES: CN(c1ccc(S(=O)(=O)N(C)C)cc1Br)C1CCCC1]

While under nitrogen, a solution of 3-bromo-4-(cyclopentylamino)-N,N-dimethylbenzene-1-sulfonamide (782 mg, 2.25 mmol) in DMF (4.5 mL) was cooled to 0° C. and treated with NaH (162 mg, 60%, 4.05 mmol). After stirring 15 min, MeI (0.21 mL, 3.38 mmol) was added and the reaction was warmed to 60° C. After stirring for 5 h, the reaction re-cooled to 0° C. and quenched with saturated aqueous ammonium chloride and extracted with ethyl acetate. The organic layer was dried over anhydrous magnesium sulfate, filtered, and concentrated. Purification by flash column chromatography (20% ethyl acetate in hexanes) gave the title compound as a white solid (0.72 g, 88% yield). ¹H NMR (400 MHz, Chloroform-d) δ 7.95-7.87 (m, 1H), 7.60 (d, J=8.4 Hz, 1H), 7.13 (s, 1H), 3.82 (s, 1H), 2.74 (s, 3H), 2.71 (s, 6H), 1.70 (s, 2H), 1.54 (d, J=17.7 Hz, 6H).

### Step 5: Synthesis of 2-(cyclopentyl(methyl)amino)-5-(N,N-dimethylsulfamoyl)-N—(S-ethylthiazol-2-yl)benzamide

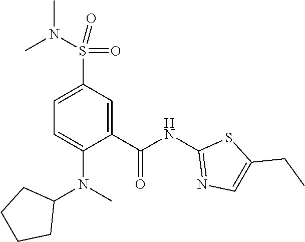 [SMILES: CCc1cnc(NC(=O)c2cc(S(=O)(=O)N(C)C)ccc2N(C)C2CCCC2)s1]

While under nitrogen, a mixture of 3-bromo-4-(cyclopentylamino)-N,N-dimethylbenzene-1-sulfonamide (110 mg, 0.305 mmol), 4,5-dimethyl-1,3-thiazol-2-amine (46.8 mg, 0.365 mmol), xantphos (8.8 mg, 0.0152 mmol) and palladium(II) acetate (3.4 mg, 0.0152 mmol) in DMSO (1 mL) was treated with triethylamine (0.06 mL, 0.457 mmol), purged with CO and stirred under CO (balloon) at 85° C. for 12 h. Once complete, the reaction was diluted with water and extracted with ethyl acetate. The organic layer was dried over anhydrous magnesium sulfate, filtered, and concentrated. Purification by preparative thin layer chromatography using a mixture of dichloromethane, methanol and saturated aqueous ammonium hydroxide (20:1:0.1) gave the title compound as an off-white solid (80 mg, 60% yield). ¹H NMR (400 MHz, Chloroform-d) δ 8.70 (d, J=2.3 Hz, 1H), 7.99-7.81 (m, 1H), 7.51 (d, J=8.4 Hz, 1H), 3.63 (s, 1H), 2.93 (s, 3H), 2.76 (s, 6H), 2.30 (s, 6H), 1.83 (s, 2H), 1.80-1.60 (m, 6H).

### Example 2: Preparation of 2-(cyclopentyl(methyl)amino)-5-(N,N-dimethylsulfamoyl)-N-(4,5-dimethylthiazol-2-yl)benzamide

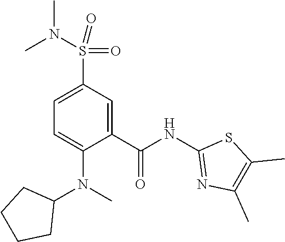 [SMILES: Cc1nc(NC(=O)c2cc(S(=O)(=O)N(C)C)ccc2N(C)C2CCCC2)sc1C]

2-(Cyclopentyl(methyl)amino)-5-(N,N-dimethylsulfamoyl)-N-(4,5-dimethylthiazol-2-yl)benzamide was prepared in a manner analogous to that set forth in Example 1, except 4,5-dimethylthiazol-2-amine was used instead of 5-ethylthiazol-2-amine in step 4. The title compound was isolated as a white solid: ¹H NMR (400 MHz, Chloroform-d) δ 8.72 (d, J=2.4 Hz, 1H), 7.93 (dd, J=8.4, 2.4 Hz, 1H), 7.53 (d, J=8.4 Hz, 1H), 7.16 (t, J=1.2 Hz, 1H), 3.59 (s, 1H), 2.86 (s, 3H), 2.81 (q, J=7.6 Hz, 2H), 2.76 (s, 6H), 1.88-1.55 (m, 8H), 1.31 (t, J=7.5 Hz, 3H).

### Example 3: Preparation of 2-((cyclopropylmethyl)(methyl)amino)-N-(5-ethylthiazol-2-yl)-5-(morpholinosulfonyl)benzamide

### Step 1: Synthesis of 2-fluoro-5-(morpholinosulfonyl)benzoic acid

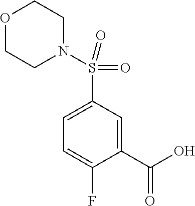 [SMILES: O=C(O)c1cc(S(=O)(=O)N2CCOCC2)ccc1F]

While under nitrogen, a solution of and 5-(chlorosulfonyl)-2-fluorobenzoic acid (2.39 g, 10 mmol) in dichloromethane (20 mL) was cooled to 0° C. and treated with triethylamine (2.78 mL, 20 mmol) and morpholine (0.88 mL, 10 mmol). After stirring for 2 days at room temperature, the reaction was concentrated, suspended in water and acidified with HCl (3N) until pH=4˜5. Extraction with ethyl acetate afforded the title compound as a white solid acid (2.44 g, 84% yield).

### Step 2: Synthesis of N-(5-ethylthiazol-2-yl)-2-fluoro-5-(morpholinosulfonyl)benzamide

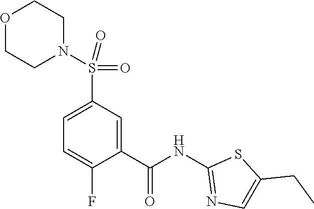 [SMILES: CCc1cnc(NC(=O)c2cc(S(=O)(=O)N3CCOCC3)ccc2F)s1]

While under nitrogen, a solution of 2-fluoro-5-(morpholine-4-sulfonyl)benzoic acid (1 g, 3.46 mmol) and 5-ethyl-1,3-thiazol-2-amine (443 mg, 3.46 mmol) in DMF (5 mL) was treated with diisopropylethylamine (0.57 mL, 3.46 mmol) and HATU (1.32 g, 3.46 mmol). After stirring for 12 h at room temperature, the reaction was diluted with ethyl acetate, washed with water, dried over anhydrous magnesium sulfate, filtered, and concentrated. Purification by flash column (30-50% ethyl acetate in hexanes) gave the title compound as a white solid (1.2 g, 87% yield). ¹H NMR (400 MHz, Chloroform-d) δ 8.53 (dd, J=6.8, 2.4 Hz, 1H), 7.97 (ddd, J=8.7, 4.6, 2.5 Hz, 1H), 7.41 (dd, J=10.8, 8.7 Hz, 1H), 7.13 (t, J=1.2 Hz, 1H), 3.73 (dd, J=5.7, 3.7 Hz, 5H), 3.07-2.98 (m, 4H), 2.82 (qd, J=7.5, 1.1 Hz, 2H), 1.32 (t, J=7.5 Hz, 3H).

### Step 3: Synthesis of 2-((cyclopropylmethyl)(methyl)amino)-N-(5-ethylthiazol-2-yl)-5-(morpholinosulfonyl)benzamide

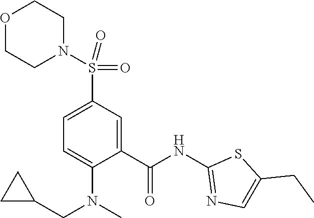 [SMILES: CCc1cnc(NC(=O)c2cc(S(=O)(=O)N3CCOCC3)ccc2N(C)CC2CC2)s1]

A solution of N-(5-ethyl-1,3-thiazol-2-yl)-2-fluoro-5-(morpholine-4-sulfonyl)benzamide (360 mg, 0.901 mmol) and 1-cyclopropyl-N-methylmethanamine hydrochloride (109 mg, 0.901 mmol) in DMSO (2 mL) was treated with and diisopropylethylamine (0.30 mL, 1.80 mmol) and warmed to 80° C. with stirring for 6 h. After cooling to room temperature, the reaction was diluted with ethyl acetate, washed with water, dried over anhydrous magnesium sulfate, filtered, and concentrated. Purification by preparative thin layer chromatography using a mixture of dichloromethane, methanol and saturated aqueous ammonium hydroxide (20:1:0.1) gave the title compound as a white solid (350 mg, 84% yield). ¹H NMR (400 MHz, Chloroform-d) δ 13.20 (s, 1H), 8.61 (d, J=2.4 Hz, 1H), 7.87 (dd, J=8.4, 2.4 Hz, 1H), 7.46 (d, J=8.5 Hz, 1H), 7.14 (t, J=1.2 Hz, 1H), 3.77-3.69 (m, 4H), 3.06-2.99 (m, 5H), 2.94 (d, J=12.8 Hz, 6H), 2.81 (qd, J=7.5, 1.2 Hz, 2H), 1.32 (t, J=7.5 Hz, 3H), 1.09-0.84 (m, 1H), 0.58-0.46 (m, 2H), 0.11 (dt, J=6.1, 4.8 Hz, 2H).

### Example 4: Preparation of 2-(cyclopentyl(methyl)amino)-N-(5-ethylthiazol-2-yl)-5-(morpholinosulfonyl)benzamide

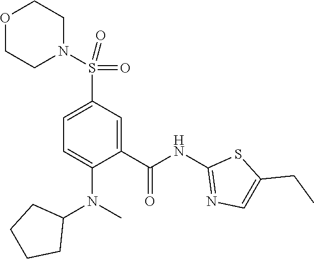 [SMILES: CCc1cnc(NC(=O)c2cc(S(=O)(=O)N3CCOCC3)ccc2N(C)C2CCCC2)s1]

2-(Cyclopentyl(methyl)amino)-N-(5-ethylthiazol-2-yl)-5-(morpholinosulfonyl)benzamide (4) was prepared in a manner analogous to that set forth in Example 3, except N-methylcyclopentanamine was used instead of 1-cyclopropyl-N-methylmethanamine in step 3. The title compound was isolated as a white solid: ¹H NMR (400 MHz, Chloroform-d) δ 8.71 (d, J=2.3 Hz, 1H), 7.90 (dd, J=8.4, 2.4 Hz, 1H), 7.54 (d, J=8.5 Hz, 1H), 7.17 (s, 1H), 3.78-3.70 (m, 4H), 3.63 (s, 1H), 3.08-3.01 (m, 4H), 2.93 (s, 2H), 2.81 (qd, J=7.5, 1.2 Hz, 2H), 2.10-1.40 (m, 6H), 1.42-1.27 (m, 3H).

### Example 5: Preparation of N-(5-ethylthiazol-2-yl)-2-(isopropyl(methyl)amino)-5-(morpholinosulfonyl)benzamide

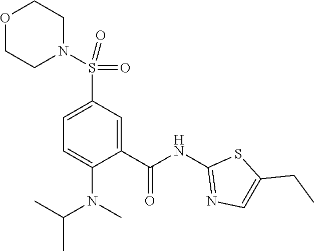 [SMILES: CCc1cnc(NC(=O)c2cc(S(=O)(=O)N3CCOCC3)ccc2N(C)C(C)C)s1]

N-(5-Ethylthiazol-2-yl)-2-(isopropyl(methyl)amino)-5-(morpholinosulfonyl)benzamide (5) was prepared in a manner analogous to that set forth in Example 3, except N-methylpropan-2-amine was used instead of 1-cyclopropyl-N-methylmethanamine in step 3. The title compound was isolated as a white solid: ¹H NMR (400 MHz, Chloroform-d) δ 13.44 (s, 1H), 8.61 (d, J=2.3 Hz, 1H), 7.88 (dd, J=8.5, 2.4 Hz, 1H), 7.41 (d, J=8.5 Hz, 1H), 7.15 (d, J=1.2 Hz, 1H), 3.78-3.70 (m, 4H), 3.55 (s, 1H), 3.08-3.00 (m, 4H), 2.92 (s, 3H), 2.81 (qd, J=7.5, 1.2 Hz, 2H), 1.35 (dt, J=23.4, 7.5 Hz, 3H), 1.19 (d, J=6.5 Hz, 6H).

### Example 6: Preparation of 2-(cyclohexyl(methyl)amino)-N-(5-ethylthiazol-2-yl)-5-(morpholinosulfonyl)benzamide

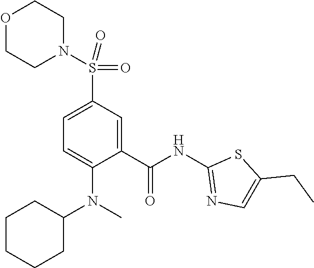 [SMILES: CCc1cnc(NC(=O)c2cc(S(=O)(=O)N3CCOCC3)ccc2N(C)C2CCCCC2)s1]

2-(Cyclohexyl(methyl)amino)-N-(5-ethylthiazol-2-yl)-5-(morpholinosulfonyl)benzamide (6) was prepared in a manner analogous to that set forth in Example 3, except N-methylcyclohexanamine was used instead of 1-cyclopropyl-N-methylmethanamine in step 3. The title compound was isolated as a white solid: ¹H NMR (400 MHz, Chloroform-d) δ 13.64 (s, 1H), 8.64 (d, J=2.3 Hz, 1H), 7.87 (dd, J=8.5, 2.4 Hz, 1H), 7.44 (d, J=8.5 Hz, 1H), 3.78-3.71 (m, 4H), 3.08-3.01 (m, 4H), 2.93 (s, 3H), 2.81 (qd, J=7.6, 1.2 Hz, 2H), 1.89 (d, J=12.1 Hz, 2H), 1.77 (d, J=13.1 Hz, 2H), 1.59 (m, 2H), 1.48-1.35 (m, 2H), 1.32 (t, J=7.5 Hz, 3H), 1.20-1.01 (m, 2H).

### Example 7: Preparation of 2-(cyclobutyl(methyl)amino)-N-(5-ethylthiazol-2-yl)-5-(morpholinosulfonyl)benzamide

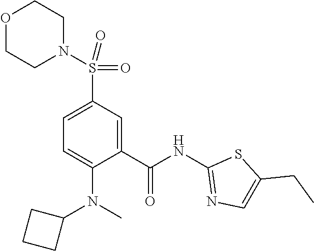 [SMILES: CCc1cnc(NC(=O)c2cc(S(=O)(=O)N3CCOCC3)ccc2N(C)C2CCC2)s1]

2-(Cyclobutyl(methyl)amino)-N-(5-ethylthiazol-2-yl)-5-(morpholinosulfonyl)benzamide (7) was prepared in a manner analogous to that set forth in Example 3, except N-methylcyclobutanamine was used instead of 1-cyclopropyl-N-methylmethanamine in step 3. The title compound was isolated as a white solid: ¹H NMR (400 MHz, Chloroform-d) δ 8.64 (d, J=2.3 Hz, 1H), 7.88 (dd, J=8.4, 2.3 Hz, 1H), 7.36 (d, J=8.5 Hz, 1H), 7.18 (s, 1H), 3.78-3.70 (m, 4H), 3.08-2.97 (m, 4H), 2.89 (s, 3H), 2.87-2.76 (m, 2H), 2.23-1.98 (m, 3H), 1.80-1.55 (m, 4H), 1.33 (t, J=7.5 Hz, 3H).

### Example 8: Preparation of 2-(allyl(methyl)amino)-N-(5-ethylthiazol-2-yl)-5-(morpholinosulfonyl)benzamide

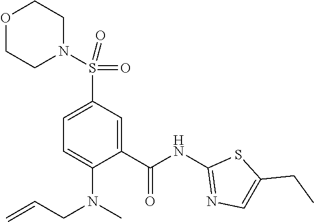 [SMILES: C=CCN(C)c1ccc(S(=O)(=O)N2CCOCC2)cc1C(=O)Nc1ncc(CC)s1]

2-(Allyl(methyl)amino)-N-(5-ethylthiazol-2-yl)-5-(morpholinosulfonyl)benzamide (8) was prepared in a manner analogous to that set forth in Example 3, except N-methylprop-2-en-1-amine was used instead of 1-cyclopropyl-N-methylmethanamine in step 3. The title compound was isolated as a white solid: ¹H NMR (400 MHz, Chloroform-d) δ 12.62 (s, 1H), 8.50 (d, J=2.3 Hz, 1H), 7.84 (dd, J=8.5, 2.3 Hz, 1H), 7.36 (d, J=8.5 Hz, 1H), 7.09 (t, J=1.1 Hz, 1H), 5.91 (ddt, J=16.8, 10.4, 6.5 Hz, 1H), 5.31-5.20 (m, 2H), 3.80-3.70 (m, 4H), 3.68 (dt, J=6.5, 1.3 Hz, 2H), 3.08-2.99 (m, 4H), 2.86 (s, 3H), 2.81 (qd, J=7.5, 1.2 Hz, 2H), 1.31 (t, J=7.5 Hz, 3H).

### Example 9: Preparation of 2-(cyclopentyl(methyl)amino)-N-(4,5-dimethylthiazol-2-yl)-5-(morpholinosulfonyl)benzamide

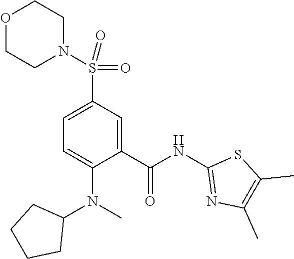 [SMILES: Cc1nc(NC(=O)c2cc(S(=O)(=O)N3CCOCC3)ccc2N(C)C2CCCC2)sc1C]

2-(Cyclopentyl(methyl)amino)-N-(4,5-dimethylthiazol-2-yl)-5-(morpholinosulfonyl)benzamide (9) was prepared in a manner analogous to that set forth in Example 3, except 4,5-dimethylthiazol-2-amine was used instead of 5-ethylthiazol-2-amine in step 2 and N-methylcyclopentanamine was used instead of 1-cyclopropyl-N-methylmethanamine in step 3. The title compound was isolated as a white solid: ¹H NMR (400 MHz, Chloroform-d) δ 13.55 (s, 1H), 8.65 (d, J=2.3 Hz, 1H), 7.86 (dd, J=8.4, 2.4 Hz, 1H), 7.49 (d, J=8.4 Hz, 1H), 3.77-3.70 (m, 4H), 3.54 (p, J=7.3 Hz, 1H), 3.07-2.98 (m, 4H), 2.81 (s, 3H), 2.30 (d, J=0.9 Hz, 3H), 2.24 (d, J=0.9 Hz, 3H), 1.88-1.68 (m, 2H), 1.67-1.48 (m, 2H), 1.19 (d, J=6.1 Hz, 4H).

### Example 10: Preparation of 2-((cyclopropylmethyl)(methyl)amino)-N-(4,5-dimethylthiazol-2-yl)-5-(morpholinosulfonyl)benzamide

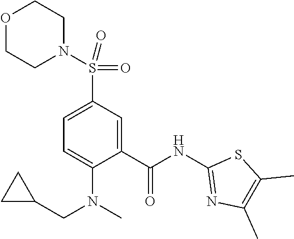 [SMILES: Cc1nc(NC(=O)c2cc(S(=O)(=O)N3CCOCC3)ccc2N(C)CC2CC2)sc1C]

2-((Cyclopropylmethyl)(methyl)amino)-N-(4,5-dimethylthiazol-2-yl)-5-(morpholinosulfonyl)benzamide (10) was prepared in a manner analogous to that set forth in Example 3, except 4,5-dimethylthiazol-2-amine was used instead of 5-ethylthiazol-2-amine in step 2. The title compound was isolated as a white solid: ¹H NMR (400 MHz, Chloroform-d) δ 12.77 (s, 1H), 8.57 (d, J=2.3 Hz, 1H), 7.85 (dd, J=8.5, 2.4 Hz, 1H), 7.43 (d, J=8.5 Hz, 1H), 3.77-3.69 (m, 4H), 3.08-2.99 (m, 4H), 2.97 (s, 3H), 2.94 (d, J=6.9 Hz, 2H), 2.30 (d, J=0.9 Hz, 3H), 2.24 (d, J=0.9 Hz, 3H), 1.03-0.83 (m, 1H), 0.56-0.46 (m, 2H), 0.09 (dt, J=6.1, 4.8 Hz, 2H).

### Example 11: Preparation of 2-(cyclohexyl(methyl)amino)-5-(N,N-dimethylsulfamoyl)-N-(5-ethylthiazol-2-yl)benzamide

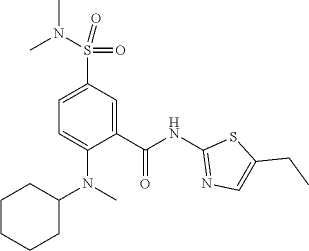 [SMILES: CCc1cnc(NC(=O)c2cc(S(=O)(=O)N(C)C)ccc2N(C)C2CCCCC2)s1]

2-(Cyclohexyl(methyl)amino)-5-(N,N-dimethylsulfamoyl)-N-(5-ethylthiazol-2-yl)benzamide (11) was prepared in a manner analogous to that set forth in Example 3, except N-methylcyclohexanamine was used instead of 1-cyclopropyl-N-methylmethanamine in step 3 and N,N-dimethylamine hydrochloride was used instead of morpholine in Step 1. The title compound was isolated as a white solid: ¹H NMR (400 MHz, Chloroform-d) δ 13.68 (s, 1H), 8.66 (d, J=2.3 Hz, 1H), 7.90 (dd, J=8.4, 2.4 Hz, 1H), 7.43 (d, J=8.4 Hz, 1H), 3.00 (s, 1H), 2.90 (s, 3H), 2.86-2.72 (m, 2H), 2.76 (s, 6H), 1.88 (d, J=12.1 Hz, 2H), 1.76 (d, J=12.8 Hz, 2H), 1.59 (d, J=11.7 Hz, 2H), 1.41 (dd, J=11.9, 3.1 Hz, 2H), 1.32 (t, J=7.5 Hz, 3H), 1.26-1.03 (m, 2H).

### Example 12: Preparation of 2-((cyclopropylmethyl)(methyl)amino)-5-(N,N-dimethylsulfamoyl)-N-(5-ethylthiazol-2-yl)benzamide

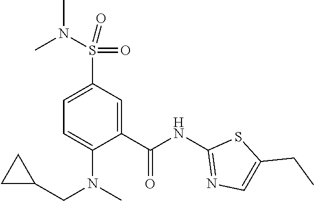 [SMILES: CCc1cnc(NC(=O)c2cc(S(=O)(=O)N(C)C)ccc2N(C)CC2CC2)s1]

2-((Cyclopropylmethyl)(methyl)amino)-5-(N,N-dimethylsulfamoyl)-N-(5-ethylthiazol-2-yl)benzamide (12) was prepared in a manner analogous to that set forth in Example 3, except N,N-dimethylamine hydrochloride was used instead of morpholine in Step 1. The title compound was isolated as a white solid: ¹H NMR (400 MHz, Chloroform-d) δ 13.45 (s, 1H), 8.67 (d, J=2.3 Hz, 1H), 7.93 (dd, J=8.4, 2.3 Hz, 1H), 7.48 (d, J=8.5 Hz, 1H), 7.14 (s, 1H), 3.01 (s, 5H), 2.86-2.70 (m, 2H), 2.75 (s, 6H), 1.32 (t, J=7.5 Hz, 3H), 0.92 (s, 1H), 0.55-0.45 (m, 2H), 0.15 (s, 2H).

### Example 13: Preparation of 5-(N,N-dimethylsulfamoyl)-2-(ethyl(methyl)amino)-N-(5-ethylthiazol-2-yl)benzamide

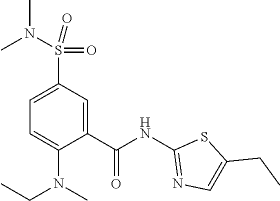 [SMILES: CCc1cnc(NC(=O)c2cc(S(=O)(=O)N(C)C)ccc2N(C)CC)s1]

5-(N,N-dimethylsulfamoyl)-2-(ethyl(methyl)amino)-N-(5-ethylthiazol-2-yl)benzamide (13) was prepared in a manner analogous to that set forth in Example 3, except N-methylethanamine was used instead of 1-cyclopropyl-N-methylmethanamine in step 3 and N,N-dimethylamine hydrochloride was used instead of morpholine in Step 1. The title compound was isolated as a white solid: ¹H NMR (400 MHz, Chloroform-d) δ 13.30 (s, 1H), 8.62 (d, J=2.3 Hz, 1H), 7.91 (dd, J=8.5, 2.4 Hz, 1H), 7.43 (d, J=8.5 Hz, 1H), 7.13 (s, 1H), 3.18 (d, J=7.9 Hz, 2H), 2.89 (s, 3H), 2.81 (qd, J=7.5, 1.0 Hz, 2H), 2.75 (s, 6H), 1.31 (t, J=7.5 Hz, 3H), 1.19 (t, J=7.2 Hz, 3H).

### Example 14: Preparation of 5-(N,N-dimethylsulfamoyl)-N-(5-ethylthiazol-2-yl)-2-(isopropyl(methyl)amino)benzamide

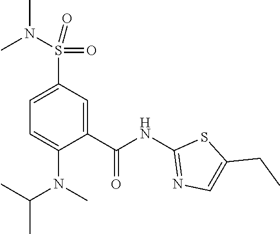 [SMILES: CCc1cnc(NC(=O)c2cc(S(=O)(=O)N(C)C)ccc2N(C)C(C)C)s1]

5-(N,N-Dimethylsulfamoyl)-N-(5-ethylthiazol-2-yl)-2-(isopropyl(methyl)amino)benzamide (14) was prepared in a manner analogous to that set forth in Example 3, except N-methylpropan-2-amine was used instead of 1-cyclopropyl-N-methylmethanamine in step 3 and N,N-dimethylamine hydrochloride was used instead of morpholine in Step 1. The title compound was isolated as a white solid: ¹H NMR (400 MHz, Chloroform-d) δ 13.50 (s, 1H), 8.66-8.55 (m, 1H), 7.91 (dd, J=8.4, 2.4 Hz, 1H), 7.40 (d, J=8.5 Hz, 1H), 7.14 (s, 1H), 3.51 (s, 1H), 2.89 (s, 3H), 2.80 (qd, J=7.5, 1.2 Hz, 2H), 2.75 (s, 6H), 1.31 (t, J=7.5 Hz, 3H), 1.18 (d, J=6.5 Hz, 6H).

### Example 15: Preparation of 2-(cyclobutyl(methyl)amino)-5-(N,N-dimethylsulfamoyl)-N-(5-ethylthiazol-2-yl)benzamide

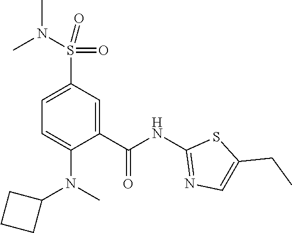 [SMILES: CCc1cnc(NC(=O)c2cc(S(=O)(=O)N(C)C)ccc2N(C)C2CCC2)s1]

2-(Cyclobutyl(methyl)amino)-5-(N,N-dimethylsulfamoyl)-N-(5-ethylthiazol-2-yl)benzamide (15) was prepared in a manner analogous to that set forth in Example 3, except N-methylcyclobutanamine was used instead of 1-cyclopropyl-N-methylmethanamine in step 3 and N,N-dimethylamine hydrochloride was used instead of morpholine in Step 1. The title compound was isolated as a white solid: ¹H NMR (400 MHz, Chloroform-d) δ 13.74 (s, 1H), 8.66 (d, J=2.3 Hz, 1H), 7.90 (dd, J=8.4, 2.3 Hz, 1H), 7.35 (d, J=8.4 Hz, 1H), 7.17 (s, 1H), 3.90-3.76 (m, 1H), 2.89-2.78 (m, 5H), 2.76 (s, 6H), 2.23-1.98 (m, 4H), 1.83-1.60 (m, 2H), 1.32 (t, J=7.5 Hz, 3H).

### Example 16: Preparation of 2-((cyclobutylmethyl)(methyl)amino)-5-(N,N-dimethylsulfamoyl)-N-(5-ethylthiazol-2-yl)benzamide

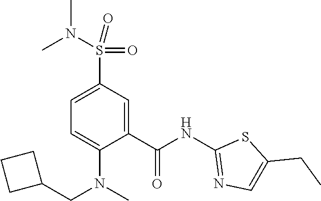 [SMILES: CCc1cnc(NC(=O)c2cc(S(=O)(=O)N(C)C)ccc2N(C)CC2CCC2)s1]

2-((Cyclobutylmethylxmethyl)amino)-5-(N,N-dimethylsulfamoyl)-N-(5-ethylthiazol-2-yl)benzamide (16) was prepared in a manner analogous to that set forth in Example 3, except 1-cyclobutyl-N-methylmethanamine was used instead of 1-cyclopropyl-N-methylmethanamine in step 3 and N,N-dimethylamine hydrochloride was used instead of morpholine in Step 1. The title compound was isolated as a white solid: ¹H NMR (400 MHz, Chloroform-d) δ 13.15 (s, 1H), 8.63 (d, J=2.4 Hz, 1H), 7.90 (dd, J=8.4, 2.4 Hz, 1H), 7.43 (d, J=8.4 Hz, 1H), 7.16 (d, J=1.2 Hz, 1H), 3.14 (d, J=7.3 Hz, 2H), 2.86-2.77 (m, 5H), 2.75 (s, 6H), 2.56 (p, J=7.7 Hz, 1H), 2.00 (dd, J=10.2, 6.9 Hz, 1H), 1.95-1.59 (m, 4H), 1.32 (t, J=7.5 Hz, 3H).

### Example 17: Preparation of 2-(cyclopentyl(methyl)amino)-5-(N,N-dimethylsulfamoyl)-N-(5-methylthiazol-2-yl)benzamide

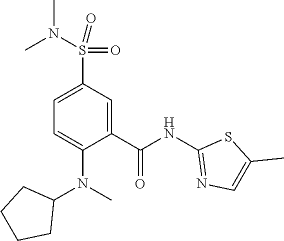 [SMILES: Cc1cnc(NC(=O)c2cc(S(=O)(=O)N(C)C)ccc2N(C)C2CCCC2)s1]

2-(Cyclopentyl(methyl)amino)-5-(N,N-dimethylsulfamoyl)-N-(5-methylthiazol-2-yl)benzamide (17) was prepared in a manner analogous to that set forth in Example 3, except N,N-dimethylamine hydrochloride was used instead of morpholine in step 1, 5-methylthiazol-2-amine was used instead of 5-ethylthiazol-2-amine in step 2, and 1-cyclopentyl-N-methylmethanamine was used instead of 1-cyclopropyl-N-methylmethanamine in step 3. The title compound was isolated as a white solid: ¹H NMR (400 MHz, Chloroform-d) δ 8.72 (d, J=2.3 Hz, 1H), 7.93 (dd, J=8.4, 2.4 Hz, 1H), 7.52 (d, J=8.5 Hz, 1H), 7.14 (d, J=1.7 Hz, 1H), 3.57 (t, J=7.4 Hz, 1H), 2.85 (s, 3H), 2.76 (s, 6H), 2.42 (d, J=1.2 Hz, 3H), 1.91-1.78 (m, 2H), 1.78-1.69 (m, 2H), 1.69-1.46 (m, 4H).

### Example 18: Preparation of 5-(N-benzyl-N-methylsulfamoyl)-2-((cyclopropylmethyl)(methyl)amino)-N-(5-ethylthiazol-2-yl)benzamide

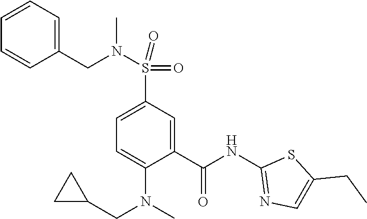 [SMILES: CCc1cnc(NC(=O)c2cc(S(=O)(=O)N(C)Cc3ccccc3)ccc2N(C)CC2CC2)s1]

5-(N-Benzyl-N-methylsulfamoyl)-2-((cyclopropylmethyl)(methyl)amino)-N-(5-ethylthiazol-2-yl)benzamide (18) was prepared in a manner analogous to that set forth in Example 3, except N-methyl-1-phenylmethanamine was used instead of morpholine in step 1. The title compound was isolated as a white solid: ¹H NMR (400 MHz, Chloroform-d) δ 13.34 (s, 1H), 8.70 (d, J=2.4 Hz, 1H), 7.96 (dd, J=8.4, 2.4 Hz, 1H), 7.46 (d, J=8.5 Hz, 1H), 7.36-7.22 (m, 5H), 7.15 (s, 1H), 4.20 (s, 2H), 2.96 (s, 3H), 2.94 (d, J=6.9 Hz, 2H), 2.87-2.76 (m, 2H), 2.64 (s, 3H), 1.32 (t, J=7.5 Hz, 3H), 0.97 (tdd, J=12.8, 7.1, 3.7 Hz, 1H), 0.58-0.48 (m, 2H), 0.12 (d, J=5.1 Hz, 2H).

### Example 19: Preparation of 2-((cyclopropylmethyl)(methyl)amino)-N-(5-methyl-4-phenylthiazol-2-yl)-5-(morpholinosulfonyl)benzamide

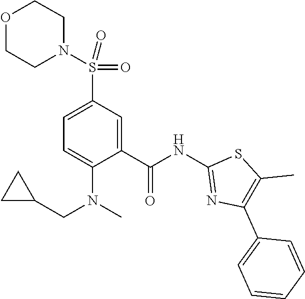 [SMILES: Cc1sc(NC(=O)c2cc(S(=O)(=O)N3CCOCC3)ccc2N(C)CC2CC2)nc1-c1ccccc1]

2-((Cyclopropylmethyl)(methyl)amino)-N-(5-methyl-4-phenylthiazol-2-yl)-5-(morpholinosulfonyl)benzamide (19) was prepared in a manner analogous to that set forth in Example 3, except 5-methyl-4-phenylthiazol-2-amine was used instead of 5-ethylthiazol-2-amine in step 2. The title compound was isolated as a white solid: ¹H NMR (400 MHz, Chloroform-d) δ 13.01 (s, 1H), 8.61 (d, J=2.4 Hz, 1H), 7.87 (dd, J=8.5, 2.4 Hz, 1H), 7.67-7.59 (m, 2H), 7.49-7.38 (m, 3H), 7.37-7.27 (m, 1H), 3.78-3.70 (m, 4H), 3.07-3.00 (m, 4H), 2.97 (s, 3H), 2.95 (d, J=6.9 Hz, 2H), 2.54 (s, 3H), 1.08-0.95 (m, 1H), 0.61-0.52 (m, 2H), 0.16 (d, J=5.1 Hz, 2H).

### Example 20: Preparation of 2-((cyclopropylmethyl)(methyl)amino)-N-(4-ethylthiazol-2-yl)-5-(morpholinosulfonyl)benzamide

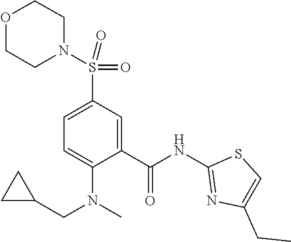 [SMILES: CCc1csc(NC(=O)c2cc(S(=O)(=O)N3CCOCC3)ccc2N(C)CC2CC2)n1]

2-((Cyclopropylmethyl)(methyl)amino)-N-(4-ethylthiazol-2-yl)-5-(morpholinosulfonyl)benzamide (20) was prepared in a manner analogous to that set forth in Example 3, except 4-ethylthiazol-2-amine was used instead of 5-ethylthiazol-2-amine in step 2. The title compound was isolated as a white solid: 1H NMR (400 MHz, Chloroform-d) δ 12.96 (s, 1H), 8.59 (d, J=2.4 Hz, 1H), 7.86 (dd, J=8.5, 2.4 Hz, 1H), 7.44 (d, J=8.5 Hz, 1H), 6.58 (s, 1H), 3.77-3.69 (m, 4H), 3.06-2.99 (m, 4H), 2.98 (s, 3H), 2.94 (d, J=6.9 Hz, 2H), 2.71 (qd, J=7.5, 1.1 Hz, 2H), 1.26 (t, J=7.5 Hz, 3H), 0.98 (qd, J=7.7, 4.1 Hz, 1H), 0.58-0.48 (m, 2H), 0.12 (q, J=5.3 Hz, 2H).

### Example 21: Preparation of 2-(cyclopentyl(methyl)amino)-5-(N,N-dimethylsulfamoyl)-N-(4,5-dimethylthiazol-2-yl)nicotinamide

### Step 1: Synthesis of 2-oxo-5-sulfo-1,2-dihydropyridine-3-carboxylic acid

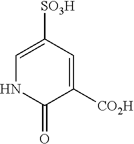 [SMILES: O=C(O)c1cc(S(=O)(=O)O)c[nH]c1=O]

2-Hydroxynicotinic acid (2.78 g, 20 mmol) was added portionwise to 26-29% fuming sulfuric acid (3 mL, 9.77 mmol) at 50° C. The resulting mixture was heated to 140° C. After 16 h, the reaction was allowed to cool to room temperature. Ice (10 g) was added and the resulting white precipitate was collected by filtration and washed with acetone. The filter cake was dried under vacuum to give the title compound as a white solid 2-oxo-5-sulfo-1,2-dihydropyridine-3-carboxylic acid (2.85 g, 65% yield). ¹H NMR (400 MHz, DMSO-d₆) δ 8.47 (s, 3H), 8.40 (d, J=2.6 Hz, 1H), 7.93 (d, J=2.6 Hz, 1H).

### Step 2: Synthesis of 5-(ethoxycarbonyl)-6-oxo-1,6-dihydropyridine-3-sulfonic acid

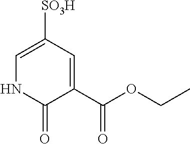 [SMILES: CCOC(=O)c1cc(S(=O)(=O)O)c[nH]c1=O]

A suspension of 2-oxo-5-sulfo-1,2-dihydropyridine-3-carboxylic acid (5.02 g, 22.9 mmol) in ethanol (50 mL) and heated to reflux. After 12 h, approximately 20 mL of ethanol was removed by distillation and fresh ethanol (20 mL) was added. Refluxing was continued for an additional 4 h and an additional 20 mL of ethanol was removed by distillation. Approximately 25 mL of toluene was added, and the solution was again heated at reflux. After 4 h, approximately 45 mL of mixed solvent was removed by distillation and the reaction was cooled to room temperature without stirring. After 12 h, the resulting off-white solid was collected by filtration and washed successively with ethyl acetate and acetone to give the title compound as an off-white solid (4.5 g, 79% yield). ¹H NMR (400 MHz, DMSO-d₆) δ 11.30 (s, 2H), 8.10 (d, J=2.6 Hz, 1H), 7.65 (d, J=2.7 Hz, 1H), 4.17 (q, J=7.1 Hz, 2H), 1.22 (t, J=7.1 Hz, 3H).

### Step 3: Synthesis of ethyl 2-chloro-5-(chlorosulfonyl)nicotinate

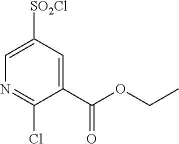 [SMILES: CCOC(=O)c1cc(S(=O)(=O)Cl)cnc1Cl]

A suspension of 5-(ethoxycarbonyl)-6-oxo-1,6-dihydropyridine-3-sulfonic acid (742 mg, 3 mmol) in thionyl chloride (4.4 mL) was treated with dimethylformamide (2 drops) and heated to reflux for 3 h. After cooling to room temperature, the excess of thionyl chloride was removed by distillation and the resulting product (colorless oil) was used directly in the next step without further purification.

### Step 4: Synthesis of ethyl 2-chloro-5-(N,N-dimethylsulfamoyl)nicotinate

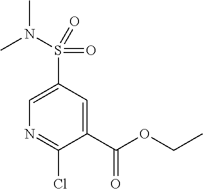 [SMILES: CCOC(=O)c1cc(S(=O)(=O)N(C)C)cnc1Cl]

A solution of ethyl 2-chloro-5-(chlorosulfonyl)pyridine-3-carboxylate (852 mg, 3 mmoL) and dimethylamine hydrochloride (245 mg, 3 mmol) in dichloromethane was cooled to 0° C. (ice-bath) and treated with triethyl amine (1.04 mL, 7.5 mmol). After 2 h, the ice-bath was removed and stirring was continued for 12 h. The resulting mixture was concentrated re-dissolved in ethyl acetate, washed successively with saturated aqueous NaHCO₃ and water. The organic layer was dried over sodium sulfate, filtered, and concentrated. Recrystallization from 20% ethyl acetate in hexanes gave the title compound as a white solid (495 mg, 56% yield over 2 steps). ¹H NMR (400 MHz, Chloroform-d) δ 8.82 (d, J=2.5 Hz, 1H), 8.42 (d, J=2.5 Hz, 1H), 4.44 (q, J=7.1 Hz, 2H), 2.78 (s, 6H), 1.41 (t, J=7.1 Hz, 3H).

### Step 5: Synthesis of ethyl 2-(cyclopentyl(methyl)amino)-5-(N,N-dimethylsulfamoyl)nicotinate

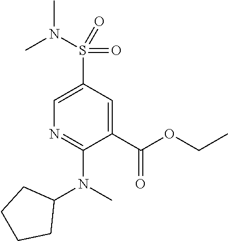 [SMILES: CCOC(=O)c1cc(S(=O)(=O)N(C)C)cnc1N(C)C1CCCC1]

A solution of N-methylcyclopentanamine hydrochloride (339 mg, 2.5 mmol) and ethyl 2-chloro-5-(dimethylsulfamoyl)-1,2-dihydropyridine-3-carboxylate (732 mg, 2.5 mmol) in dioxane (5 mL) was treated with triethylamine (0.76 mL, 5.5 mmol) and warmed to 60° C. for 4 h. After cooling to room temperature, the reaction was concentrated, re-dissolved in ethyl acetate, washed with water, dried over sodium sulfate, filtered and concentrated to give the title compound as a yellow oil (0.88 g, 99% yield). ¹H NMR (400 MHz, Chloroform-d) δ 8.50 (d, J=2.4 Hz, 1H), 8.14 (d, J=2.4 Hz, 1H), 4.89 (p, J=8.4 Hz, 1H), 4.33 (q, J=7.1 Hz, 2H), 2.80 (s, 3H), 2.71 (s, 6H), 2.06-1.87 (m, 2H), 1.77-1.51 (m, 6H), 1.36 (t, J=7.1 Hz, 3H).

### Step 6: Synthesis of 2-(cyclopentyl(methyl)amino)-5-(N,N-dimethylsulfamoyl)nicotinic acid

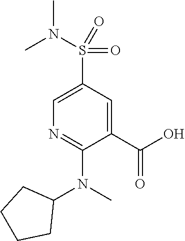 [SMILES: CN(c1ncc(S(=O)(=O)N(C)C)cc1C(=O)O)C1CCCC1]

A solution of ethyl 2-[cyclopentyl(methyl)amino]-5-(dimethylsulfamoyl)pyridine-3-carboxylate (880 mg, 2.47 mmol) in ethanol (5 mL) was treated with aqueous 2N NaOH (2.5 mL, 2N, 5 mmol) and stirred at 80° C. for 5 h. After cooling to room temperature, the reaction mixture was quenched with aqueous HCl (3 mL, 6N in dioxane, 18 mmol) and concentrated give the title compound as a white solid that was used in the subsequent step without further purification.

### Step 7: Synthesis of 2-(cyclopentyl(methyl)amino)-5-(N,N-dimethylsulfamoyl)-N-(4,5-dimethylthiazol-2-yl)nicotinamide

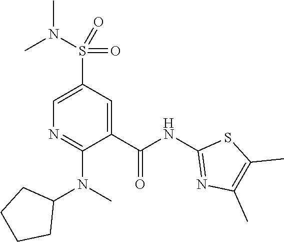 [SMILES: Cc1nc(NC(=O)c2cc(S(=O)(=O)N(C)C)cnc2N(C)C2CCCC2)sc1C]

While under nitrogen, a solution of 2-[cyclopentyl(methyl)amino]-N-(dimethyl-1,3-thiazol-2-yl)-5-(dimethylsulfamoyl)pyridine-3-carboxamide (40 mg, 0.122 mmol), 4,5-dimethyl-1,3-thiazol-2-amine (17.2 mg, 0.134 mmol) and diisopropylethylamine (19.0 mg, 0.147 mmol) in DMF (0.5 mL) was treated with HATU (55.7 mg, 0.147 mmol) and warmed to 80° C. After 3 h, the reaction was cooled to room temperature, diluted with ethyl acetate, washed with water, dried over sodium sulfate, filtered, and concentrated. Purification by preparative thin layer chromatography using a mixture of dichloromethane, methanol and saturated aqueous ammonium hydroxide (20:1:0.1) gave the title compound as an off-white solid (25 mg, 47% yield). ¹H NMR (400 MHz, Chloroform-d) δ 8.63 (d, J=2.4 Hz, 1H), 8.30 (d, J=2.5 Hz, 1H), 4.37 (p, J=7.9 Hz, 1H), 2.88 (s, 3H), 2.71 (s, 6H), 2.28 (d, J=0.9 Hz, 3H), 2.10-2.00 (m, 3H), 1.98-1.79 (m, 2H), 1.76-1.47 (m, 6H).

### Example 22: Preparation of 2-((cyclopropylmethyl)(methyl)amino)-5-(N,N-dimethylsulfamoyl)-N-(5-ethylthiazol-2-yl)nicotinamide

### Step 1: Synthesis of 5-bromo-6-chloro-N,N-dimethylpyridine-3-sulfonamide

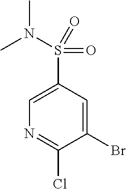

A solution of dimethylamine hydrochloride (257 mg, 3.15 mmol) and 5-bromo-6-chloropyridine-3-sulfonyl chloride (873 mg, 3 mmol) in dichloromethane (20 mL) was cooled to 0° C. (ice-bath) and treated with triethylamine (0.94 mL, 6.75 mmol). After stirring for 10 min, the ice bath was removed and stirring was continued for 4 h at room temperature. The resulting reaction mixture was concentrated, diluted with ethyl acetate, and washed with water. The organic layer was dried over sodium sulfate, filtered and concentrated to give the title compound as a white solid (0.88 g. 98% yield). ¹H NMR (400 MHz, Chloroform-d) δ 8.68 (d, J=2.2 Hz, 1H), 8.26 (d, J=2.2 Hz, 1H), 2.79 (s, 6H).

### Step 2: Synthesis of 5-bromo-6-((cyclopropylmethyl)(ethyl)amino)-N,N-dimethylpyridine-3-sulfonamide

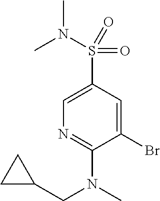 [SMILES: CN(CC1CC1)c1ncc(S(=O)(=O)N(C)C)cc1Br]

A solution of 5-bromo-6-chloro-N,N-dimethylpyridine-3-sulfonamide (420 mg, 1.40 mmol), (cyclopropylmethyl)(methyl)amine hydrochloride (171 mg, 1.40 mmol) in DMSO (2 mL) was treated with diisopropylethylamine (0.51 mL, 3.08 mmol) and warmed to 80° C. After stirring for 12 h, the reaction was cooled to room temperature, diluted with ethyl acetate and washed with water. The organic layer was dried over sodium sulfate, filtered and concentrated. Purification by flash column (20% ethyl acetate in hexanes) gave the title compound as a white solid (430 mg, 88% yield). ¹H NMR (400 MHz, Chloroform-d) δ 8.46 (d, J=2.1 Hz, 1H), 8.02 (d, J=2.2 Hz, 1H), 3.44 (d, J=6.7 Hz, 2H), 3.22 (s, 3H), 2.72 (s, 6H), 1.19-1.05 (m, 1H), 0.60-0.49 (m, 2H), 0.22 (dt, J=6.2, 4.7 Hz, 2H).

### Step 3: Synthesis of 2-((cyclopropylmethyl)(methyl)amino)-5-(N,N-dimethylsulfamoyl)-N-(5-ethylthiazol-2-yl)nicotinamide

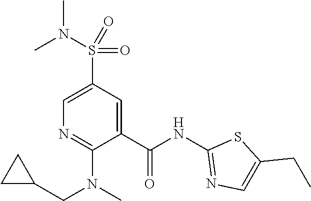 [SMILES: CCc1cnc(NC(=O)c2cc(S(=O)(=O)N(C)C)cnc2N(C)CC2CC2)s1]

While under nitrogen, a mixture of 2-[(cyclopropylmethyl)(methyl)amino]-5-(dimethylsulfamoyl)-N-(5-ethyl-1,3-thiazol-2-yl)pyridine-3-carboxamide (60 mg, 0.172 mmol), 2-[(cyclopropylmethyl)(methyl)amino]-5-(dimethylsulfamoyl)-N-(5-ethyl-1,3-thiazol-2-yl)pyridine-3-carboxamide (24.3 mg, 0.189 mmol), xantphos (5.0 mg, 0.0086 mmol) and palladium(II) acetate (1.9 mg, 0.0086 mmol) was dissolved in DMSO (1 mL) and triethylamine (0.04 mL, 0.258 mmol). The resulting solution was purged with CO and stirred under CO (balloon) at 70° C. for 6 h. After cooling to room temperature, the reaction mixture was diluted with water and extracted with ethyl acetate. The organic layer was dried over sodium sulfate, filtered and concentrated. Purification by preparative thin layer chromatography using a mixture of dichloromethane, methanol and saturated aqueous ammonium hydroxide (20:1:0.1) gave the title compound as an off-white solid (39 mg, 53% yield). ¹H NMR (400 MHz, Chloroform-d) δ 8.67 (d, J=2.4 Hz, 1H), 8.29 (d, J=2.4 Hz, 1H), 6.76 (s, 1H), 3.51-3.44 (m, 2H), 3.11 (s, 3H), 2.81-2.67 (m, 2H), 2.74 (s, 6H), 1.29 (t, J=7.5 Hz, 3H), 1.09-0.93 (m, 1H), 0.55-0.44 (m, 2H), 0.28-0.17 (m, 2H).

### Example 23: Preparation of 2-((cyclopropylmethyl)(methyl)amino)-5-(N,N-dimethylsulfamoyl)-N-(4,5-dimethylthiazol-2-yl)nicotinamide

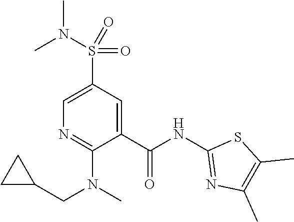 [SMILES: Cc1nc(NC(=O)c2cc(S(=O)(=O)N(C)C)cnc2N(C)CC2CC2)sc1C]

2-((Cyclopropylmethyl)(methyl)amino)-5-(N,N-dimethylsulfamoyl)-N-(4,5-dimethylthiazol-2-yl)nicotinamide (23) was prepared in a manner analogous to that set forth in Example 22, except 4,5-dimethylthiazol-2-amine was used instead of 5-ethylthiazol-2-amine in step 3. The title compound was isolated as a white solid: ¹H NMR (400 MHz, Chloroform-d) δ 8.61 (d, J=2.5 Hz, 1H), 8.24 (d, J=2.4 Hz, 1H), 3.38 (d, J=6.8 Hz, 2H), 3.09 (s, 3H), 2.69 (s, 6H), 2.27 (d, J=0.9 Hz, 3H), 2.04 (s, 2H), 1.10-0.90 (m, 1H), 0.56-0.46 (m, 2H), 0.18 (dt, J=6.0, 4.7 Hz, 2H).

### Example 24: Preparation of 2-((cyclopropylmethyl)(methyl)amino)-N-(6,7-dihydro-4H-pyrano[4,3-d]thiazol-2-yl)-5-(N,N-dimethylsulfamoyl)nicotinamide

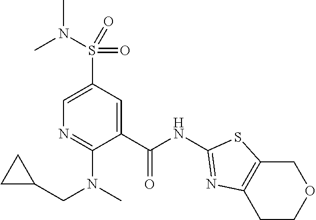 [SMILES: CN(CC1CC1)c1ncc(S(=O)(=O)N(C)C)cc1C(=O)Nc1nc2c(s1)COCC2]

2-((Cyclopropylmethyl)(methyl)amino)-N-(6,7-dihydro-4H-pyrano[4,3-d]thiazol-2-yl)-5-(N,N-dimethylsulfamoyl)nicotinamide (24) was prepared in a manner analogous to that set forth in Example 22, except 6,7-dihydro-4H-pyrano[4,3-d]thiazol-2-amine was used instead of 5-ethylthiazol-2-amine in step 3. The title compound was isolated as a white solid: ¹H NMR (400 MHz, Chloroform-d) δ 8.67 (d, J=2.4 Hz, 1H), 8.38 (d, J=2.4 Hz, 1H), 4.77 (t, J=1.8 Hz, 2H), 3.99 (t, J=5.5 Hz, 2H), 3.39 (d, J=6.9 Hz, 2H), 3.11 (s, 3H), 2.74 (s, 6H), 2.75-2.65 (m, 2H), 1.07-0.96 (m, 1H), 0.59-0.46 (m, 2H), 0.19 (dt, J=6.1, 4.7 Hz, 2H).

### Example 25: Preparation of 2-(cyclopentyl(methyl)amino)-5-(N,N-dimethylsulfamoyl)-N-(5-ethylthiazol-2-yl)nicotinamide

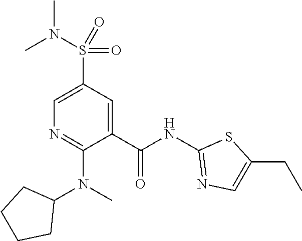 [SMILES: CCc1cnc(NC(=O)c2cc(S(=O)(=O)N(C)C)cnc2N(C)C2CCCC2)s1]

2-(Cyclopentyl(methyl)amino)-5-(N,N-dimethylsulfamoyl)-N-(5-ethylthiazol-2-yl)nicotinamide (25) was prepared in a manner analogous to that set forth in Example 22, except N-methylcyclopentanamine was used instead of (cyclopropylmethyl)(methyl)amine hydrochloride in step 2. The title compound was isolated as a white solid: ¹H NMR (400 MHz, Chloroform-d) δ 8.66 (d, J=2.4 Hz, 1H), 8.23 (d, J=2.4 Hz, 1H), 6.65 (s, 1H), 4.56 (p, J=7.9 Hz, 1H), 2.85 (s, 3H), 2.73 (s, 6H), 2.80-2.64 (m, 2H), 1.83 (s, 2H), 1.74-1.47 (m, 6H), 1.28 (t, J=7.5 Hz, 3H).

### Example 26: Preparation of 2-((cyclopropylmethyl)(methyl)amino)-N-(5-ethylthiazol-2-yl)-5-(morpholinosulfonyl)nicotinamide

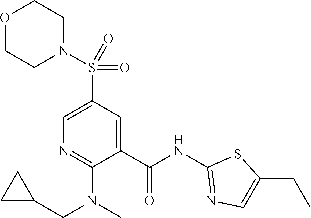 [SMILES: CCc1cnc(NC(=O)c2cc(S(=O)(=O)N3CCOCC3)cnc2N(C)CC2CC2)s1]

2-((Cyclopropylmethyl)(methyl)amino)-N-(5-ethylthiazol-2-yl)-5-(morpholinosulfonyl)nicotinamide (26) was prepared in a manner analogous to that set forth in Example 22, except morpholine was used instead of dimethylamine hydrochloride in step 1. The title compound was isolated as a white solid: ¹H NMR (400 MHz, Chloroform-d) δ 8.59 (t, J=1.7 Hz, 1H), 8.14 (d, J=2.4 Hz, 1H), 6.69 (s, 1H), 3.73 (t, J=4.6 Hz, 4H), 3.42 (d, J=6.8 Hz, 2H), 3.08 (s, 3H), 3.01 (t, J=4.8 Hz, 4H), 2.76 (q, J=7.6 Hz, 2H), 1.28 (t, J=7.5 Hz, 3H), 1.11-1.01 (m, 1H), 0.54 (t, J=6.8 Hz, 2H), 0.21 (d, J=5.1 Hz, 2H).

### Example 27: Preparation of 3-((cyclopropylmethyl)(methyl)amino)-N-(5-ethylthiazol-2-yl)-6-(morpholinosulfonyl)pyridazine-4-carboxamide

### Step 1: Synthesis of methyl 3,6-dichloropyridazine-4-carboxylate

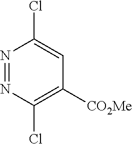 [SMILES: COC(=O)c1cc(Cl)nnc1Cl]

A solution of 3,6-dichloropyridazine-4-carboxylic acid (1.93 g, 10 mmol) in dichloromethane (10 mL) and MeOH (3 mL) was cooled to 0° C. and treated with (trimethylsilyl)diazomethane (2M in diethylether, 7.5 mL, 15 mmol). After stirring at room temperature for 2 h, the reaction was diluted with ethyl acetate, washed with water, and concentrated to give the title compound as a pale-yellow oil (2.0 g, 97% yield) which was used without further purification.

### Step 2: Synthesis of methyl 6-chloro-3-((cyclopropylmethyl)(methyl)amino)pyridazine-4-carboxylate

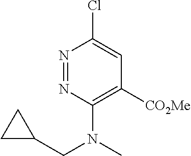 [SMILES: COC(=O)c1cc(Cl)nnc1N(C)CC1CC1]

A solution of methyl 3,6-dichloropyridazine-4-carboxylate (2.0 g, 9.661 mmol), 1-cyclopropyl-N-methylmethanamine (1.18 g, 9.66 mmol) in acetonitrile (25 mL) was treated with diisopropylethylamine (3.2 mL, 19.3 mmol) and stirred at 25° C. for 24 h. The resulting reaction mixture was diluted with ethyl acetate and washed with water, dried over sodium sulfate, filtered, and concentrated to give the title compound as a bright yellow oil (2.4 g, 93% yield) which was used without further purification.

### Step 3: Synthesis of methyl 6-(benzylthio)-3-((cyclopropylmethyl)(methy)amino)pyridazine-4-carboxylate

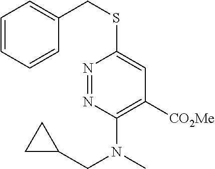 [SMILES: COC(=O)c1cc(SCc2ccccc2)nnc1N(C)CC1CC1]

A suspension of NaH (60%, 56 mg, 1.39 mmol) in anhydrous THF (1 mL) was cooled to at 0° C. (ice-bath) and treated with benzyl mercaptan (150 mg, 1.21 mmol). After stirring at 0° C. for 0.5 h, methyl 6-chloro-3-[(cyclopropylmethyl)(methyl)amino]pyridazine-4-carboxylate (238 mg, 0.929 mmol) was added and ice-bath was removed. After stirring for 2 h the reaction mixture was warmed to 70° C. for 12 h. Once complete, the reaction mixture was cooled to room temperature, diluted with ethyl acetate, washed with water, dried over sodium sulfate, filtered, and concentrated. Purification by flash column (10% ethyl acetate in hexanes) gave the title compound as a bright yellow oil (180 mg, 56% yield). ¹H NMR (400 MHz, Chloroform-d) δ 7.38 (d, J=7.5 Hz, 2H), 7.32-7.17 (m, 5H), 4.51 (s, 2H), 3.89 (d, J=1.2 Hz, 3H), 3.34 (d, J=6.7 Hz, 2H), 3.07 (d, J=1.2 Hz, 3H), 1.15-1.03 (m, 1H), 0.59-0.49 (m, 2H), 0.21 (t, J=5.3 Hz, 2H).

### Step 4: Synthesis of methyl 6-(chlorosulfonyl)-3-((cyclopropylmethyl)(methyl)amino) pyridazine-4-carboxylate

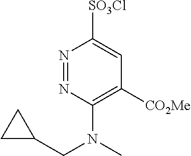

A solution of methyl 6-(benzylsulfanyl)-3-[(cyclopropylmethyl)(methyl)amino]pyridazine-4-carboxylate (290 mg, 0.844 mmol) in acetic acid (1 mL) and water (0.5 mL) was cooled to 0° C. and carefully treated with N-chlorosuccinimide (451 mg, 3.38 mmol). After the addition was complete stirring was continued at 25° C. for 2 h. The resulting solution was concentrated, diluted with water and extracted with ethyl acetate to give the title compound as a yellow oil (250 mg, 93% yield) that was used without further purification.

### Step 5: Synthesis of methyl 3-((cyclopropylmethyl)(methyl)amino)-6-(morpholinosulfonyl)pyridazine-4-carboxylate

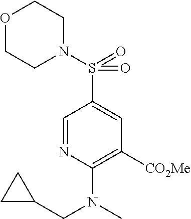 [SMILES: COC(=O)c1cc(S(=O)(=O)N2CCOCC2)cnc1N(C)CC1CC1]

A solution of methyl 6-(chlorosulfonyl)-3-[(cyclopropylmethyl)(methyl)amino]pyridazine-4-carboxylate (250 mg, 0.782 mmol) and morpholine (68 mg, 0.782 mmol) in dichloromethane (5 mL) was cooled to 0° C. (ice-bath) and treated with triethylamine (0.12 mL, 0.86 mmol). After stirring 16 h at room temperature, the reaction was concentrated, diluted with ethyl acetate and washed with water. The organic layer was dried over sodium sulfate, filtered and concentrated. Purification by preparative thin layer chromatography using a mixture of dichloromethane, methanol and saturated aqueous ammonium hydroxide (20:1:0.1) gave the title compound as a yellow solid (185 mg, 64% yield). ¹H NMR (400 MHz, Chloroform-d) δ 7.94 (s, 1H), 3.93 (s, 3H), 3.76 (t, J=4.7 Hz, 4H), 3.64 (d, J=6.8 Hz, 2H), 3.38 (t, J=4.7 Hz, 4H), 3.12 (s, 3H), 1.21-1.10 (m, 1H), 0.59 (p, J=6.6, 6.1 Hz, 2H), 0.31 (t, J=5.2 Hz, 2H).

### Step 6: Synthesis of 3-((cyclopropylmethyl)(methyl)amino)-6-(morpholinosulfonyl) pyridazine-4-carboxylic acid

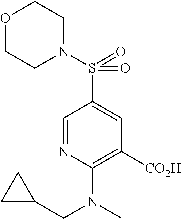 [SMILES: CN(CC1CC1)c1ncc(S(=O)(=O)N2CCOCC2)cc1C(=O)O]

A solution of methyl 3-[(cyclopropylmethyl)(methyl)amino]-6-(morpholine-4-sulfonyl)pyridazine-4-carboxylate (130 mg, 0.351 mmol) in THF (1 mL) and MeOH (1 mL) was cooled to 0° C. and treated with aqueous LiOH (0.7 mL, 1N, 0.70 mmol). After 2 h at RT, the reaction was neutralized with 1N HCl (0.7 mL) and concentrated to give the title compound as a yellow solid that was used without further purification.

### Step 7: Synthesis of 3-((cyclopropylmethyl)(methyl)amino)-N-(5-ethylthiazol-2-yl)-6-(morpholinosulfonyl)pyridazine-4-carboxamide

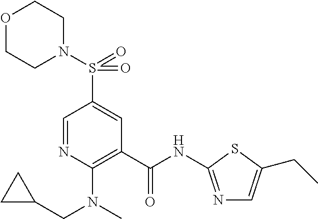 [SMILES: CCc1cnc(NC(=O)c2cc(S(=O)(=O)N3CCOCC3)cnc2N(C)CC2CC2)s1]

A solution of 3-[(cyclopropylmethyl)(methyl)amino]-6-(morpholine-4-sulfonyl)pyridazine-4-carboxylic acid (80 mg, 0.225 mmol) in dichloromethane was cooled to 0° C. and treated with oxalyl chloride (0.05 mL, 0.561 mmol) and N,N-dimethylformamide (2 drops). After stirring for 2 h, the reaction mixture was concentrated under vacuum. The resulting oil was re-dissolved in dichloromethane (1 mL) and treated with a second solution of 5-ethyl-1,3-thiazol-2-amine (34.5 mg, 0.269 mmol) and triethylamine (0.15 mL, 1.12 mmol) in dichloromethane (1 mL) at 0° C. The resulting mixture was stirred at room temperature for 5 h, concentrated, diluted with ethyl acetate, washed with water, dried over sodium sulfate, filtered and concentrated. Purification by preparative thin layer chromatography using a mixture of dichloromethane, methanol and saturated aqueous ammonium hydroxide (20:1:0.1) to give the title compound as a yellow solid (25 mg, 24% yield). ¹H NMR (400 MHz, Chloroform-d) δ 12.91 (s, 1H), 7.89 (s, 1H), 6.57 (s, 1H), 3.76 (dd, J=5.9, 3.6 Hz, 4H), 3.62 (d, J=6.8 Hz, 2H), 3.44-3.36 (m, 4H), 3.18 (s, 3H), 2.80-2.69 (m, 2H), 1.27 (t, J=7.5 Hz, 3H), 1.20-1.06 (m, 1H), 0.61-0.51 (m, 2H), 0.27 (d, J=5.1 Hz, 2H).

### Example 28: Preparation of 3-((cyclopropylmethyl)(methyl)amino)-N-(5-ethylthiazol-2-yl)-6-(morpholinosulfonyl)picolinamide

### Step 1: Synthesis of 2-(benzylthio)-5-bromopyridine

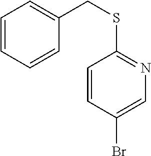 [SMILES: Brc1ccc(SCc2ccccc2)nc1]

A suspension of NaH (60%, 677 mg, 16.7 mmol) in THF (5 mL) was cooled to 0° C. (ice-bath) and treated with benzyl mercaptan (1.78 g, 14.3 mmol). After stirring for 30 min, 5-bromo-2-chloropyridine (2.29 g, 11.9 mmol) was added and stirring was continued for 1 h at room temperature then at 70° C. for 5 h. After cooling to room temperature, the reaction was diluted with dichloromethane, washed with water, dried over sodium sulfate, filtered and concentrated to give the title compound as a light yellow oil (3.28 g, 98% yield) that was used without further purification.

### Step 2: Synthesis of 5-bromopyridine-2-sulfonyl chloride

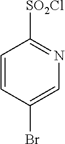 [SMILES: O=S(=O)(Cl)c1ccc(Br)cn1]

A solution of 2-(benzylsulfanyl)-5-bromopyridine (2.1 g, 7.49 mmol) in acetic acid (10 mL) and water (5 mL) was cooled to 0° C. and carefully treated with N-chlorosuccinimide (4.0 g, 30.0 mmol). After stirring for 2 h at room temperature, the solution was concentrated, diluted with ethyl acetate and washed with water to give the title compound as a yellow oil (1.9 g, 99% yield) that was used without further purification.

### Step 3: Synthesis of 4-((5-bromopyridin-2-yl)sulfonyl)morpholine

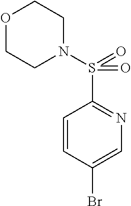 [SMILES: O=S(=O)(c1ccc(Br)cn1)N1CCOCC1]

A solution of 5-bromopyridine-2-sulfonyl chloride (192 mg, 7.5 mmol) and triethylamine (1.6 mL, 11.3 mmol) in dichloromethane (10 mL) was cooled to 0° C. (ice-bath) and treated with morpholine (849 mg, 9.75 mmol). After the addition was complete, the ice bath was removed and stirring was continued for 16 h at room temperature. The resulting mixture was concentrated, diluted with ethyl acetate, washed successively with water and saturated aqueous sodium chloride, dried over sodium sulfate, filtered and concentrated. The crude product was purified by flash column (30% ethyl acetate in hexanes) to give the title compound as a white solid (2.2 g, 95% yield). ¹H NMR (400 MHz, Chloroform-d) δ 8.75 (d, J=2.2 Hz, 1H), 8.04 (dd, J=8.3, 2.2 Hz, 1H), 7.81 (d, J=8.3 Hz, 1H), 3.76-3.69 (m, 4H), 3.30 (t, J=4.8 Hz, 4H).

### Step 4: Synthesis of N-(cyclopropylmethyl)-6-(morpholinosulfonyl)pyridin-3-amine

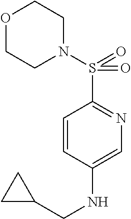 [SMILES: O=S(=O)(c1ccc(NCC2CC2)cn1)N1CCOCC1]

While under nitrogen, a solution of 4-[(5-bromopyridin-2-yl)sulfonyl]morpholine (400 mg, 1.30 mmol), potassium tert-butoxide (175.4 mg, 1.56 mmol), 1-cyclopropyl-N-methylmethanamine (101.9 mg, 1.43 mmol), xantphos (22.6 mg, 0.0392 mmol) and tris(dibenzylideneacetone)dipalladium(0) (17.9 mg, 0.0195 mmol) in toluene (5 mL) was heated at 105° C. for 16 h. After cooling to room temperature, the reaction mixture was diluted with ethyl acetate, washed successively with water and saturated aqueous sodium chloride, dried over sodium sulfate, filtered and concentrated to give the title compound as a colorless oil (380 mg, 98% yield) that was used without further purification. ¹H NMR (400 MHz, Chloroform-d) δ 8.02 (d, J=2.8 Hz, 1H), 7.70 (d, J=8.6 Hz, 1H), 7.41-7.34 (m, 1H), 3.71 (t, J=4.7 Hz, 4H), 3.21 (t, J=4.7 Hz, 4H), 2.34 (s, 2H), 1.10 (s, 1H), 0.61 (d, J=7.9 Hz, 2H), 0.28 (d, J=5.0 Hz, 2H).

### Step 5: Synthesis of 2-bromo-N-(cyclopropylmethyl)-6-(morpholinosulfonyl)pyridin-3-amine

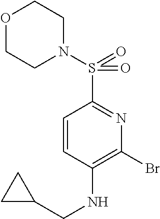 [SMILES: O=S(=O)(c1ccc(NCC2CC2)c(Br)n1)N1CCOCC1]

A solution of N-(cyclopropylmethyl)-6-(morpholine-4-sulfonyl)pyridin-3-amine (380 mg, 1.28 mmol) in DMF (5 mL) was treated with and N-bromosuccinimide (227.4 mg, 1.28 mmol) and stirred at 25° C. for 14 h. The reaction mixture was diluted with ethyl acetate, washed successively with water and saturated aqueous sodium chloride, dried over sodium sulfate, filtered and concentrated to give the title to give a brown oil (454 mg, 94% yield). ¹H NMR (400 MHz, Chloroform-d) δ 7.73-7.60 (m, 1H), 6.78 (d, J=8.4 Hz, 1H), 3.73 (q, J=4.6 Hz, 4H), 3.26 (dd, J=5.8, 3.8 Hz, 4H), 2.75 (s, 2H), 1.13 (d, J=7.3 Hz, 1H), 0.70-0.61 (m, 2H), 0.29 (dd, J=13.1, 5.2 Hz, 2H).

### Step 6: Synthesis of 2-bromo-N-(cyclopropylmethyl)-N-methyl-6-(morpholinosulfonyl)pyridin-3-anine

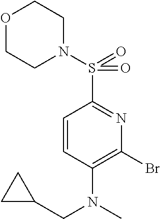 [SMILES: CN(CC1CC1)c1ccc(S(=O)(=O)N2CCOCC2)nc1Br]

A solution of 2-bromo-N-(cyclopropylmethyl)-6-(morpholine-4-sulfonyl)pyridin-3-amine (450 mg, 1.196 mmol) in DMF (2.5 mL) was cooled to 0° C. and treated with NaH (140 mg, 3.59 mmol). After stirring 15 min, iodomethane (0.11 mL, 1.79 mmol) was added, the ice-bath was removed, and the mixture was warmed to 60° C. for 5 h. After cooling to room temperature, the reaction mixture was diluted with ethyl acetate, washed successively with water and saturated aqueous sodium chloride, dried over sodium sulfate, filtered and concentrated. The crude product was purified by flash column (20% ethyl acetate in hexanes) to give the title compound as a colorless oil (220 mg, 47% yield). ¹H NMR (400 MHz, Chloroform-d) δ 7.76 (d, J=8.2 Hz, 1H), 7.34 (dd, J=21.4, 7.8 Hz, 1H), 3.79-3.70 (m, 4H), 3.43-3.24 (m, 4H), 3.09 (d, J=6.7 Hz, 2H), 2.96 (s, 3H), 1.01 (dddd, J=11.5, 8.1, 6.6, 2.4 Hz, 1H), 0.59-0.48 (m, 2H), 0.15 (dt, J=6.1, 4.7 Hz, 2H).

### Step 7: Synthesis of 3-((cyclopropylmethyl)(methyl)amino)-N—(S-ethylthiazol-2-yl)-6-(morpholinosulfonyl)picolinamide

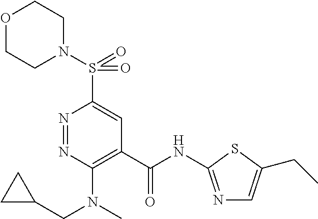 [SMILES: CCc1cnc(NC(=O)c2cc(S(=O)(=O)N3CCOCC3)nnc2N(C)CC2CC2)s1]

While under nitrogen, a solution of DMSO (2 mL) and triethylamine (0.05 mL, 0.3843 mmol) was added to a mixture of 2-bromo-N-(cyclopropylmethyl)-N-methyl-6-(morpholine-4-sulfonyl)pyridin-3-amine (100 mg, 0.256 mmol) and 5-ethyl-1,3-thiazol-2-amine (36.1 mg, 0.282 mmol), xantphos (7.4 mg, 0.0128 mmol) and Pd(OAc)₂ (2.9 mg, 0.0128 mmol). The resulting solution was purged twice with CO and stirred under CO (balloon) at 75° C. for 12 h. After cooling to room temperature, the reaction mixture was diluted with ethyl acetate, washed successively with water and saturated aqueous sodium chloride, dried over sodium sulfate, filtered and concentrated. Purification by preparative thin layer chromatography using a mixture of dichloromethane, methanol and saturated aqueous ammonium hydroxide (20:1:0.1) gave the title compound as a yellow solid (56 mg, 47% yield). ¹H NMR (400 MHz, Chloroform-d) δ 10.51 (s, 1H), 7.84 (d, J=8.9 Hz, 1H), 7.45 (d, J=8.9 Hz, 1H), 7.15 (d, J=1.1 Hz, 1H), 3.73 (dd, J=5.8, 3.6 Hz, 4H), 3.30-3.23 (m, 4H), 3.21 (d, J=6.6 Hz, 2H), 3.08 (s, 3H), 2.81 (qd, J=7.5, 1.1 Hz, 2H), 1.32 (t, J=7.5 Hz, 3H), 1.03 (t, J=6.4 Hz, OH), 0.64-0.53 (m, 2H), 0.19 (q, J=5.3 Hz, 2H).

### Example 29: Preparation of 5-(N,N-dimethylsulfamoyl)-N-(4,5-dimethylthiazol-2-yl)-3-fluoro-2-(pyrrolidin-1-yl)benzamide

### Step 1: Synthesis of 3,4-difluoro-N,N-dimethylbenzenesulfonamide

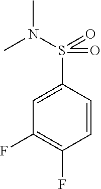

A solution of 3,4-difluorobenzenesulfonyl chloride (6.37 g, 30 mmol) and dimethylamine hydrochloride (2.57 g, 31.5 mmol) in dichloromethane was cooled to 0° C. and treated with triethylamine (9.2 mL, 66 mmol). After stirring for 4 h, the reaction was concentrated, diluted with ethyl acetate, washed successively with water and saturated aqueous sodium chloride, dried over sodium sulfate, filtered and concentrated to give the title compound as a white solid (6.4 g, 96% yield) that was used without further purification.

### Step 2: Synthesis of 3-fluoro-N,N-dimethyl-4-(pyrrolidin-1-yl)benzenesulfonamide

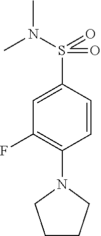 [SMILES: CN(C)S(=O)(=O)c1ccc(N2CCCC2)c(F)c1]

A solution of 3,4-difluoro-N,N-dimethylbenzene-1-sulfonamide (1.24 g, 5.60 mmol) and triethylamine (1.7 mL, 12.3 mmol) in 1,4-dioxane (5 mL) was treated with pyrrolidine (0.55 mL, 6.72 mmol) and heated to 100° C. After 4 h, the reaction was cooled to room temperature and concentrated. The resulting residue was diluted with ethyl acetate, washed successively with water and saturated aqueous sodium chloride, dried over sodium sulfate, filtered and concentrated to give the title compound as a brown solid (1.4 g, 92% yield). ¹H NMR (400 MHz, Chloroform-d) δ 7.42-7.26 (m, 2H), 6.62 (t, J=8.5 Hz, 1H), 3.50 (h, J=3.1 Hz, 4H), 2.65 (s, 6H), 2.03-1.90 (m, 4H).

### Step 3: Synthesis of 3-bromo-5-fluoro-N,N-dimethyl-4-(pyrrolidin-1-yl)benzenesulfonamide

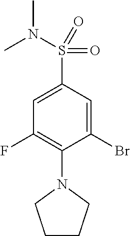 [SMILES: CN(C)S(=O)(=O)c1cc(F)c(N2CCCC2)c(Br)c1]

A solution of 3-fluoro-N,N-dimethyl-4-(pyrrolidin-1-yl)benzene-1-sulfonamide (1.4 g, 5.14 mmol) in trifluoroacetic acid (4 mL) was treated with and N-bromosuccinimide (1.19 g, 6.68 mmol). After stirred at 25° C. for 12 h, the reaction diluted with ethyl acetate, washed successively with water, saturated aqueous NaHCO₃ and saturated aqueous sodium chloride, dried over sodium sulfate, filtered and concentrated to give the title compound as a brown solid (1.6 g, 89% yield). ¹H NMR (400 MHz, Chloroform-d) δ 7.70 (dd, J=2.1, 1.4 Hz, 1H), 7.34 (dd, J=12.0, 2.2 Hz, 1H), 3.49 (tt, J=3.9, 1.6 Hz, 4H), 2.72 (s, 6H), 2.00-1.88 (m, 4H).

### Step 4: Synthesis of 5-(N,N-dimethylsulfamoyl)-N-(4,5-dimethylthiazol-2-yl)-3-fluoro-2-(pyrrolidin-1-yl)benzamide

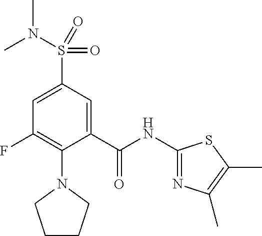 [SMILES: Cc1nc(NC(=O)c2cc(S(=O)(=O)N(C)C)cc(F)c2N2CCCC2)sc1C]

While under nitrogen, DMSO (0.6 mL) and triethylamine (0.06 mL, 0.427 mmol) were added to a mixture of 3-bromo-5-fluoro-N,N-dimethyl-4-(pyrrolidin-1-yl)benzene-1-sulfonamide (100 mg, 0.285 mmol) and 4,5-dimethyl-1,3-thiazol-2-amine (43.8 mg, 0.3417 mmol), xantphos (8.2 mg, 0.0142 mmol) and Pd(OAc)₂ (3.2 mg, 0.0142 mmol). The solution was purged twice with CO and stirred under CO (balloon) at 85° C. for 12 h. After cooling to room temperature, the reaction was cooled to room temperature, concentrated, diluted with ethyl acetate, washed successively with water and saturated aqueous sodium chloride, dried over sodium sulfate, filtered and concentrated. Purification by preparative thin layer chromatography using a mixture of dichloromethane, methanol and saturated aqueous ammonium hydroxide (20:1:0.1) gave the title compound a white solid (65 mg, 54% yield). ¹H NMR (400 MHz, Chloroform-d) δ 8.36 (dd, J=2.2, 1.1 Hz, 1H), 7.65 (dd, J=11.4, 2.2 Hz, 1H), 3.46 (q, J=5.6 Hz, 4H), 2.77 (s, 6H), 2.30 (d, J=0.9 Hz, 3H), 2.29-2.24 (m, 3H), 2.21 (td, J=5.3, 3.9, 2.1 Hz, 4H).

### Example 30: Preparation of 2-((cyclopropylmethyl)(methyl)amino)-N-(5-ethylthiazol-2-yl)-4-fluoro-5-(morpholinosulfonyl)benzamide

### Step 1: Synthesis of 4-((4-bromo-2-fluorophenyl)sulfonyl)morpholine

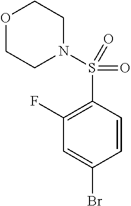 [SMILES: O=S(=O)(c1ccc(Br)cc1F)N1CCOCC1]

A solution of 4-bromo-2-fluorobenzene-1-sulfonyl chloride (5.47 g, 20 mmol) and triethylamine (6.1 mL, 44 mmol) in dichloromethane (50 mL) was cooled to 0° C. (ice-bath) and treated with morpholine (1.74 g, 20 mmol). After stirred at room temperature for 4 h, the reaction was concentrated, diluted with ethyl acetate, washed successively with water and saturated aqueous sodium chloride, dried over sodium sulfate, filtered and concentrated to give the title compound as a white solid (5.8 g, 89% yield) that was used without further purification.

### Step 2: Synthesis of N-(cyclopropylmethyl)-3-fluoro-4-(morpholinosulfonyl)aniline

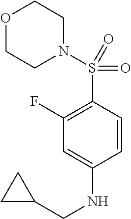 [SMILES: O=S(=O)(c1ccc(NCC2CC2)cc1F)N1CCOCC1]

While under nitrogen, a mixture of 4-(4-bromo-2-fluorobenzenesulfonyl)morpholine (1200 mg, 3.702 mmol), potassium tert-butoxide (494 mg, 4.44 mmol), cyclopropylmethanamine (289.6 mg, 4.07 mmol), xantphos (64.2 mg, 0.111 mmol) and tris(dibenzylideneacetone)dipalladium(0) (50.8 mg, 0.0555 mmol) in toluene (14 mL) was heated to 105° C. for 16 h. After stirred at room temperature, the reaction was diluted with ethyl acetate, washed successively with water and saturated aqueous sodium chloride, dried over sodium sulfate, filtered and concentrated. Purification by flash column (20% ethyl acetate in hexanes) gave the title compound as a colorless oil (1.08 g, 93% yield). ¹H NMR (400 MHz, Chloroform-d) δ 7.50 (t, J=8.3 Hz, 1H), 6.34 (dd, J=8.7, 2.3 Hz, 1H), 6.27 (dd, J=12.9, 2.3 Hz, 1H), 4.47 (s, 1H), 3.76-3.64 (m, 4H), 3.12-3.05 (m, 4H), 2.97 (dd, J=7.0, 5.0 Hz, 2H), 1.15-1.00 (m, 1H), 0.64-0.53 (m, 2H), 0.26 (dt, J=6.1, 4.7 Hz, 2H).

### Step 3: Synthesis of 2-bromo-N-(cyclopropylmethyl)-5-fluoro-4-(morpholinosulfonyl) aniline

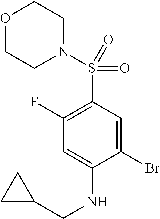 [SMILES: O=S(=O)(c1cc(Br)c(NCC2CC2)cc1F)N1CCOCC1]

While under nitrogen, a solution of N-(cyclopropylmethyl)-3-fluoro-4-(morpholine-4-sulfonyl)aniline (1.05 g, 3.34 mmol) in DMF (5 mL) was treated with N-bromosuccinimide (595 mg, 3.34 mmol). After stirring for 14 h at room temperature, the reaction mixture was diluted with ethyl acetate, washed successively with water and saturated aqueous sodium chloride, dried over sodium sulfate, filtered and concentrated to give the title compound as a white solid (1.23 g, 94% yield). ¹H NMR (400 MHz, Chloroform-d) δ 7.80 (d, J=7.2 Hz, 1H), 6.31 (d, J=12.5 Hz, 1H), 5.03 (s, 1H), 3.77-3.64 (m, 4H), 3.20-3.07 (m, 4H), 3.02 (dd, J=7.0, 4.9 Hz, 2H), 1.13 (tt, J=7.8, 4.9 Hz, 1H), 0.69-0.54 (m, 2H), 0.37-0.23 (m, 2H).

### Step 4: Synthesis of 2-bromo-N-(cyclopropylmethyl)-5-fluoro-N-methyl-4-(morpholinosulfonyl)aniline

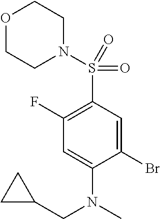 [SMILES: CN(CC1CC1)c1cc(F)c(S(=O)(=O)N2CCOCC2)cc1Br]

A solution of 2-bromo-N-(cyclopropylmethyl)-5-fluoro-4-(morpholine-4-sulfonyl)aniline (980 mg, 2.49 mmol) in DMF (5 mL) was cooled to 0° C. (ice-bath) and treated with NaH (200 mg, 60%, 4.98 mmol). After stirring for 15 min, iodomethane (0.232 mL, 3.74 mmol) was added and the ice bath was removed and the warmed to 60° C. with stirring for 3 h. The reaction was diluted with ethyl acetate, washed successively with water and saturated aqueous sodium chloride, dried over sodium sulfate, filtered and concentrated. Purification by flash column (20% ethyl acetate in hexanes) gave the title compound as a colorless oil (0.88 g, 87% yield). ¹H NMR (400 MHz, Chloroform-d) δ 7.90 (d, J=7.5 Hz, 1H), 6.78 (d, J=12.2 Hz, 1H), 3.77-3.65 (m, 4H), 3.20-3.11 (m, 4H), 3.08 (d, J=6.6 Hz, 2H), 2.95 (s, 3H), 1.08-0.96 (m, 1H), 0.59-0.48 (m, 2H), 0.14 (dt, J=5.9, 4.7 Hz, 2H).

### Step 5: Synthesis of 2-((cyclopropylmethyl)(methyl)amino)-N—(S-ethylthiazol-2-yl)-4-fluoro-5-(morpholinosulfonyl)benzamide

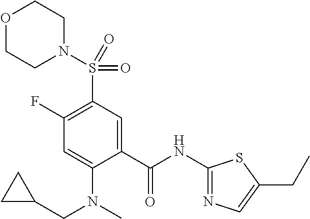 [SMILES: CCc1cnc(NC(=O)c2cc(S(=O)(=O)N3CCOCC3)c(F)cc2N(C)CC2CC2)s1]

While under nitrogen, a solution of DMSO (2 mL) and triethylamine (0.051 mL, 0.368 mmol) were added to a mixture of 2-bromo-N-(cyclopropylmethyl)-5-fluoro-N-methyl-4-(morpholine-4-sulfonyl)aniline (100 mg, 0.246 mmol) and 5-ethyl-1,3-thiazol-2-amine (31.5 mg, 0.2246 mmol), xantphos (7.4 mg, 0.0128 mmol) and Pd(OAc)₂ (2.9 mg, 0.0128 mmol) under nitrogen. The solution was purged twice with CO and stirred under CO (balloon) at 75° C. for 12 h. After cooling to room temperature, the reaction was diluted with ethyl acetate, washed with water, and concentrated. Purification by preparative thin layer chromatography using a mixture of dichloromethane, methanol and saturated aqueous ammonium hydroxide (20:1:0.1) gave the title compound as a white solid (65 mg, 55% yield). ¹H NMR (400 MHz, Chloroform-d) δ 12.33 (s, 1H), 8.59 (d, J=8.1 Hz, 1H), 7.10 (d, J=1.1 Hz, 1H), 7.06 (d, J=11.5 Hz, 1H), 3.77-3.70 (m, 4H), 3.17 (dt, J=5.5, 2.5 Hz, 4H), 2.96 (s, 3H), 2.81 (qd, J=7.5, 1.1 Hz, 2H), 1.31 (t, J=7.5 Hz, 3H), 1.05-0.90 (m, 1H), 0.60-0.51 (m, 2H), 0.13 (dt, J=6.0, 4.8 Hz, 2H).

### Example 31: Preparation of 2-((cyclopropylmethyl)(methyl)amino)-N-(4,5-dimethylthiazol-2-yl)-4-fluoro-5-(morpholinosulfonyl)benzamide

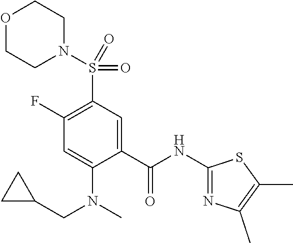 [SMILES: Cc1nc(NC(=O)c2cc(S(=O)(=O)N3CCOCC3)c(F)cc2N(C)CC2CC2)sc1C]

2-((Cyclopropylmethyl)(methyl)amino)-N-(4,5-dimethylthiazol-2-yl)-4-fluoro-5-(morpholinosulfonyl)benzamide (31) was prepared in a manner analogous to that set forth in Example 31, except 4,5-dimethylthiazol-2-amine was used instead of 5-ethylthiazol-2-amine in step 5. The title compound was isolated as a white solid: ¹H NMR (400 MHz, Chloroform-d) δ 11.85 (s, 1H), 8.55 (d, J=8.1 Hz, 1H), 7.04 (d, J=11.6 Hz, 1H), 3.73 (dd, J=5.8, 3.6 Hz, 4H), 3.16 (t, J=4.5 Hz, 4H), 2.96 (s, 3H), 2.95 (d, J=6.7 Hz, 2H), 2.33-2.27 (m, 3H), 2.22 (d, J=0.9 Hz, 3H), 0.96 (m, 1H), 0.59-0.49 (m, 2H), 0.14-0.07 (m, 2H).

### Example 32: Preparation of 5-(N,N-dimethylsulfamoyl)-N-(4,5-dimethylthiazol-2-yl)-4-fluoro-2-(pyrrolidin-1-yl)benzamide

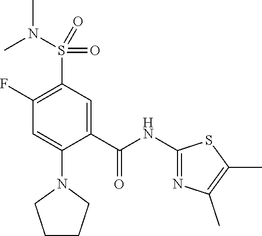 [SMILES: Cc1nc(NC(=O)c2cc(S(=O)(=O)N(C)C)c(F)cc2N2CCCC2)sc1C]

5-(N,N-Dimethylsulfamoyl)-N-(4,5-dimethylthiazol-2-yl)-4-fluoro-2-(pyrrolidin-1-yl)benzamide (32) was prepared in a manner analogous to that set forth in Example 30, except dimethylamine hydrochloride was used instead of morpholine in step 1 and 5-dimethylthiazol-2-amine was used instead of 5-ethylthiazol-2-amine in step 5. The title compound was isolated as a white solid: ¹H NMR (400 MHz, Chloroform-d) δ 7.84 (d, J=7.9 Hz, 1H), 6.44 (d, J=13.6 Hz, 1H), 3.31-3.21 (m, 4H), 2.74 (s, 3H), 2.74 (s, 3H), 2.29-2.23 (m, 3H), 1.97-1.92 (s, 4H), 1.93 (s, 3H).

### Evaluation

All HL compounds were tested in CHO-ER-LucT cells. IC₅₀ (AC₅₀) was estimated in following method: Concentration X is converted to logarithm (log₁₀) to generate sigmoidal shape curve and dose response curve is generated using Nonlinear regression (curve fit) in Prism. EC₅₀ (AC₅₀) is calculated using log(agonist) vs response-(three parameters)] which uses standard Hill slope (=1).

Max activity: [compound's fold change−1.0 (DMSO)]/[NGI−1 fold change−1.0 (DMSO)]*100(%) (Eq. 1)

TABLE 2
SAR Summary

| | | AC₅₀ | Max activity |
|---|---|---|---|
| Compound | Structure | (uM) | (% NGI-1) |
| (NGI-1) Comparator | 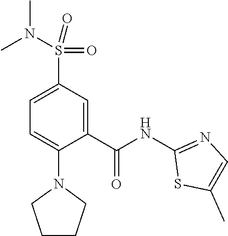 [SMILES: Cc1cnc(NC(=O)c2cc(S(=O)(=O)N(C)C)ccc2N2CCCC2)s1] | 0.7124 (D54) 1.114 (CHO) | 100 |
| | 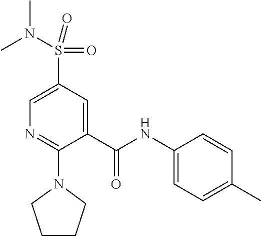 | 94.517 | 14.880 |
| | 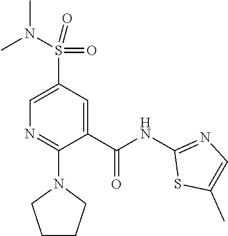 [SMILES: Cc1cnc(NC(=O)c2cc(S(=O)(=O)N(C)C)cnc2N2CCCC2)s1] | 70.420 | 50.408 |
| 32 | 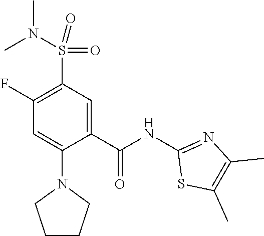 | 3.838 | 52.6 |
| 1 | 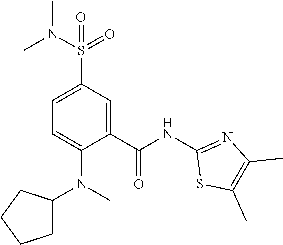 [SMILES: Cc1nc(NC(=O)c2cc(S(=O)(=O)N(C)C)ccc2N(C)C2CCCC2)sc1C] | 0.376 | 81.0 |
| 29 | 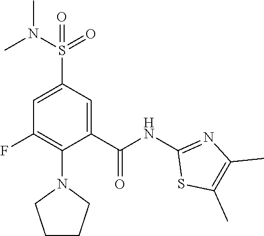 | 4.991 | 62.6 |
| | 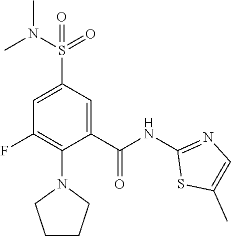 [SMILES: Cc1cnc(NC(=O)c2cc(S(=O)(=O)N(C)C)cc(F)c2N2CCCC2)s1] | 36.941 | 114.2 |
| | 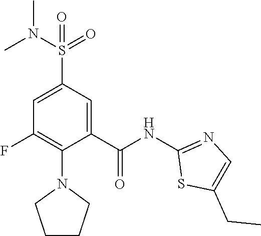 [SMILES: CCc1cnc(NC(=O)c2cc(S(=O)(=O)N(C)C)cc(F)c2N2CCCC2)s1] | 3.756 | 97.3 |
| 2 | 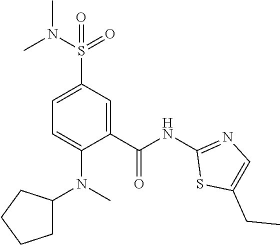 [SMILES: CCc1cnc(NC(=O)c2cc(S(=O)(=O)N(C)C)ccc2N(C)C2CCCC2)s1] | 0.120 | 128.6 |
| | 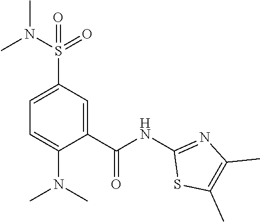 | 27.898 | 33.9 |
| | 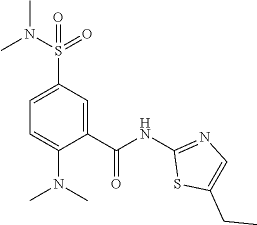 [SMILES: CCc1cnc(NC(=O)c2cc(S(=O)(=O)N(C)C)ccc2N(C)C)s1] | 6.854 | 46.1 |
| 11 | 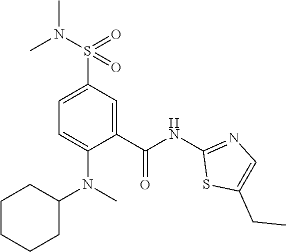 [SMILES: CCc1cnc(NC(=O)c2cc(S(=O)(=O)N(C)C)ccc2N(C)C2CCCCC2)s1] | 0.2483 | 52.9 |
| 12 | 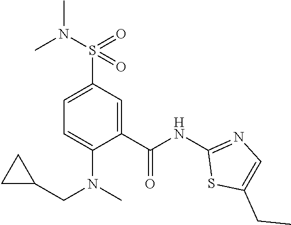 [SMILES: CCc1cnc(NC(=O)c2cc(S(=O)(=O)N(C)C)ccc2N(C)CC2CC2)s1] | 0.077 | 133 |
| 13 | 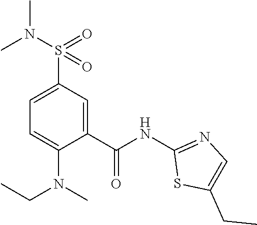 [SMILES: CCc1cnc(NC(=O)c2cc(S(=O)(=O)N(C)C)ccc2N(C)CC)s1] | 0.294 | 114 |
| Comparator | 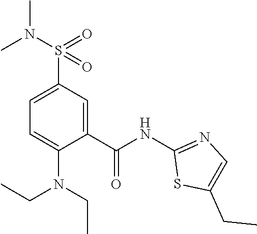 [SMILES: CCc1cnc(NC(=O)c2cc(S(=O)(=O)N(C)C)ccc2N(CC)CC)s1] | 3.356 | 46.8 |
| 14 | 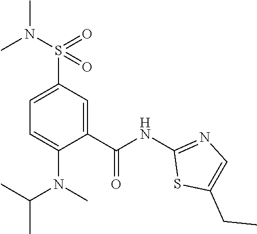 [SMILES: CCc1cnc(NC(=O)c2cc(S(=O)(=O)N(C)C)ccc2N(C)C(C)C)s1] | 0.372 | 127.8 |
| 15 | 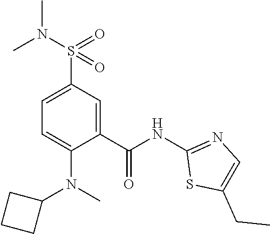 [SMILES: CCc1cnc(NC(=O)c2cc(S(=O)(=O)N(C)C)ccc2N(C)C2CCC2)s1] | 0.252 | 141 |
| 17 | 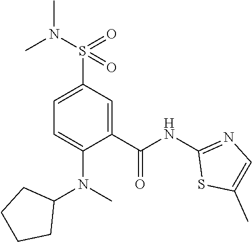 [SMILES: Cc1cnc(NC(=O)c2cc(S(=O)(=O)N(C)C)ccc2N(C)C2CCCC2)s1] | 0.361 | 102 |
| | 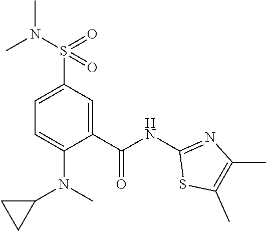 [SMILES: Cc1nc(NC(=O)c2cc(S(=O)(=O)N(C)C)ccc2N(C)C2CC2)sc1C] | 3.443 | 43.9 |
| | 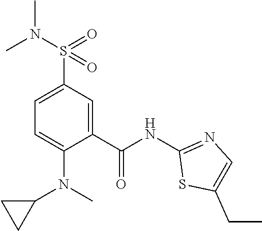 [SMILES: CCc1cnc(NC(=O)c2cc(S(=O)(=O)N(C)C)ccc2N(C)C2CC2)s1] | 1.189 | 86.8 |
| 21 | 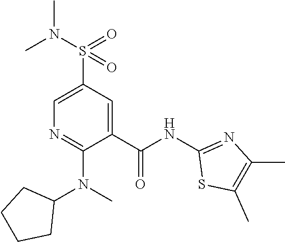 [SMILES: Cc1nc(NC(=O)c2cc(S(=O)(=O)N(C)C)cnc2N(C)C2CCCC2)sc1C] | 0.9703 | 100.7 |
| 25 | 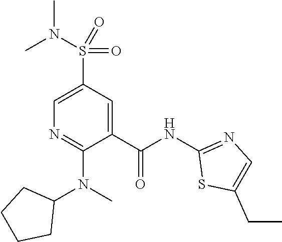 [SMILES: CCc1cnc(NC(=O)c2cc(S(=O)(=O)N(C)C)cnc2N(C)C2CCCC2)s1] | 0.3092 | 108.1 |
| | 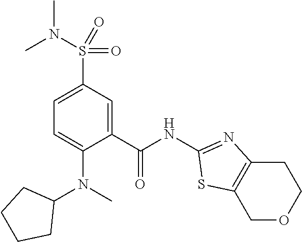 [SMILES: CN(c1ccc(S(=O)(=O)N(C)C)cc1C(=O)Nc1nc2c(s1)COCC2)C1CCCC1] | 1.02 | 46.2 |
| 22 | 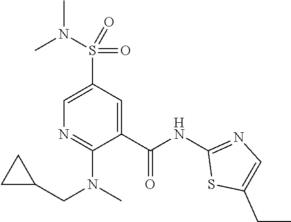 [SMILES: CCc1cnc(NC(=O)c2cc(S(=O)(=O)N(C)C)cnc2N(C)CC2CC2)s1] | 0.572 | 126.2 |
| | 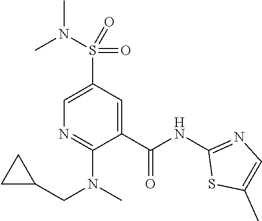 [SMILES: Cc1cnc(NC(=O)c2cc(S(=O)(=O)N(C)C)cnc2N(C)CC2CC2)s1] | 3.02 | 48.3 |
| 3 | 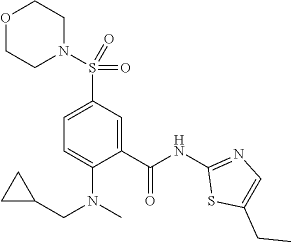 [SMILES: CCc1cnc(NC(=O)c2cc(S(=O)(=O)N3CCOCC3)ccc2N(C)CC2CC2)s1] | 0.059 | 214.6 |
| 23 | 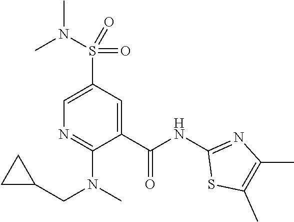 [SMILES: Cc1nc(NC(=O)c2cc(S(=O)(=O)N(C)C)cnc2N(C)CC2CC2)sc1C] | 0.863 | 59 |
| 24 | 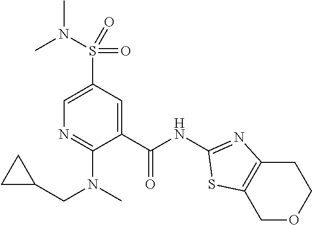 [SMILES: CN(CC1CC1)c1ncc(S(=O)(=O)N(C)C)cc1C(=O)Nc1nc2c(s1)COCC2] | 17.341 | 43.0 |
| 4 | 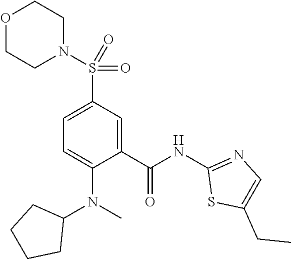 [SMILES: CCc1cnc(NC(=O)c2cc(S(=O)(=O)N3CCOCC3)ccc2N(C)C2CCCC2)s1] | 0.087 | 205.7 |
| | 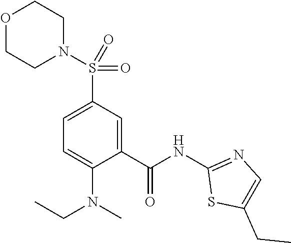 [SMILES: CCc1cnc(NC(=O)c2cc(S(=O)(=O)N3CCOCC3)ccc2N(C)CC)s1] | 0.620 | 129.5 5 |
| | 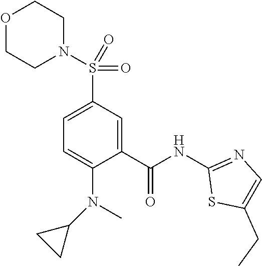 [SMILES: CCc1cnc(NC(=O)c2cc(S(=O)(=O)N3CCOCC3)ccc2N(C)C2CC2)s1] | 2.495 | 129.4 |
| 5 | 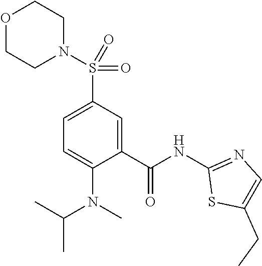 [SMILES: CCc1cnc(NC(=O)c2cc(S(=O)(=O)N3CCOCC3)ccc2N(C)C(C)C)s1] | 0.27 | 162.4 |
| 6 | 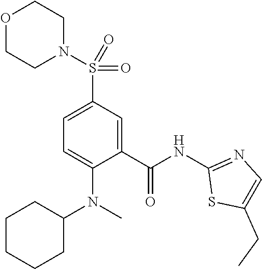 [SMILES: CCc1cnc(NC(=O)c2cc(S(=O)(=O)N3CCOCC3)ccc2N(C)C2CCCCC2)s1] | 0.2146 | 148.3 |
| 7 | 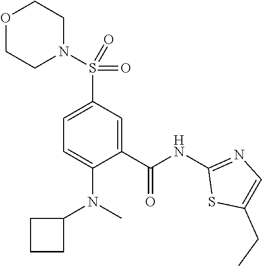 [SMILES: CCc1cnc(NC(=O)c2cc(S(=O)(=O)N3CCOCC3)ccc2N(C)C2CCC2)s1] | 0.2432 | 138 |
| | 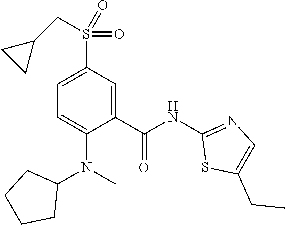 [SMILES: CCc1cnc(NC(=O)c2cc(S(=O)(=O)CC3CC3)ccc2N(C)C2CCCC2)s1] | 1.53 | 89.9 |
| | 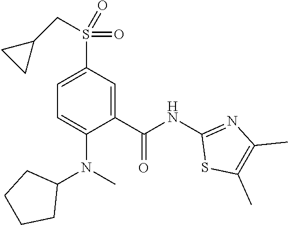 [SMILES: Cc1nc(NC(=O)c2cc(S(=O)(=O)CC3CC3)ccc2N(C)C2CCCC2)sc1C] | 2.43 | 41 |
| 9 | 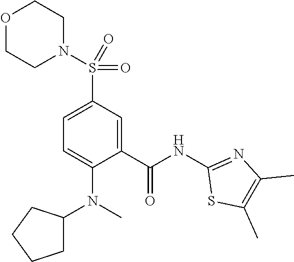 [SMILES: Cc1nc(NC(=O)c2cc(S(=O)(=O)N3CCOCC3)ccc2N(C)C2CCCC2)sc1C] | 0.1648 | 83.5 |
| 10 | 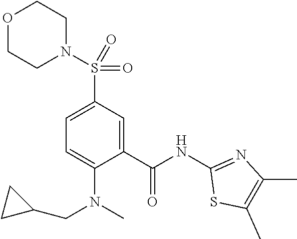 [SMILES: Cc1nc(NC(=O)c2cc(S(=O)(=O)N3CCOCC3)ccc2N(C)CC2CC2)sc1C] | 0.1282 | 88.7 |
| | 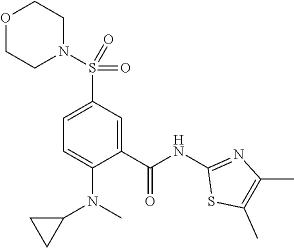 [SMILES: Cc1nc(NC(=O)c2cc(S(=O)(=O)N3CCOCC3)ccc2N(C)C2CC2)sc1C] | 2.534 | 36.7 |
| | 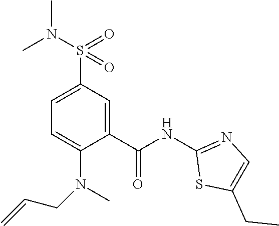 [SMILES: C=CCN(C)c1ccc(S(=O)(=O)N(C)C)cc1C(=O)Nc1ncc(CC)s1] | 1.099 | 108.5 |
| 8 | 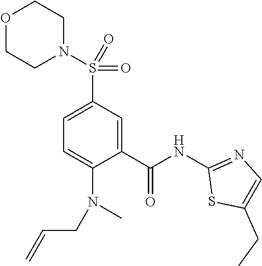 [SMILES: C=CCN(C)c1ccc(S(=O)(=O)N2CCOCC2)cc1C(=O)Nc1ncc(CC)s1] | 0.4457 | 102.3 |
| | 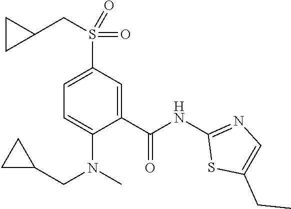 [SMILES: CCc1cnc(NC(=O)c2cc(S(=O)(=O)CC3CC3)ccc2N(C)CC2CC2)s1] | 2.593 | 88.3 |
| 18 | 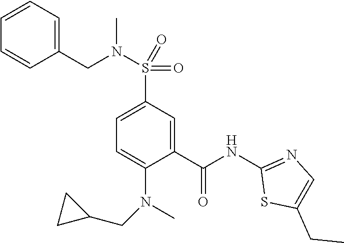 [SMILES: CCc1cnc(NC(=O)c2cc(S(=O)(=O)N(C)Cc3ccccc3)ccc2N(C)CC2CC2)s1] | 0.266 | 71.2 |
| | 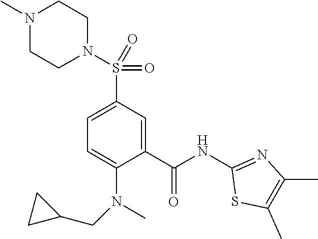 | 2.195 | 76.9 |
| 19 | 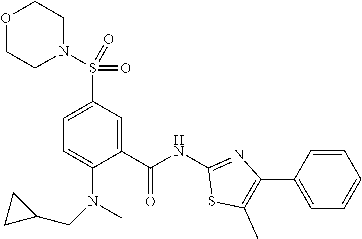 [SMILES: Cc1sc(NC(=O)c2cc(S(=O)(=O)N3CCOCC3)ccc2N(C)CC2CC2)nc1-c1ccccc1] | 0.563 | 66.2 |
| 20 | 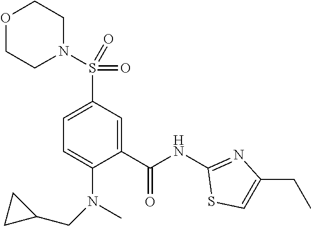 [SMILES: CCc1csc(NC(=O)c2cc(S(=O)(=O)N3CCOCC3)ccc2N(C)CC2CC2)n1] | 0.548 | 94.2 |
| | 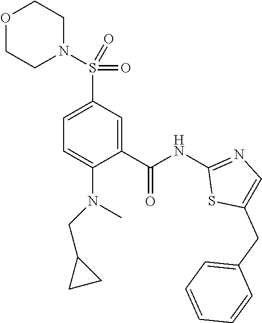 [SMILES: CN(CC1CC1)c1ccc(S(=O)(=O)N2CCOCC2)cc1C(=O)Nc1ncc(Cc2ccccc2)s1] | 13.04 | 116.5 |
| 16 | 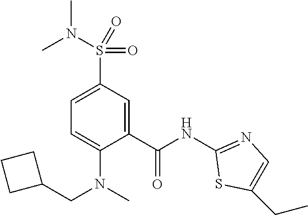 [SMILES: CCc1cnc(NC(=O)c2cc(S(=O)(=O)N(C)C)ccc2N(C)CC2CCC2)s1] | 0.744 | 82.2 |
| 26 | 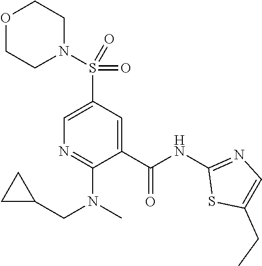 [SMILES: CCc1cnc(NC(=O)c2cc(S(=O)(=O)N3CCOCC3)cnc2N(C)CC2CC2)s1] | 0.851 | 104.4 |
| 27 | 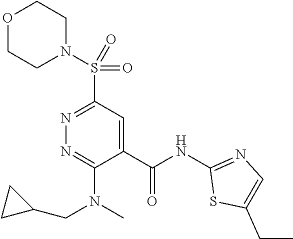 [SMILES: CCc1cnc(NC(=O)c2cc(S(=O)(=O)N3CCOCC3)nnc2N(C)CC2CC2)s1] | 0.115 | 13.3 |
| 28 | 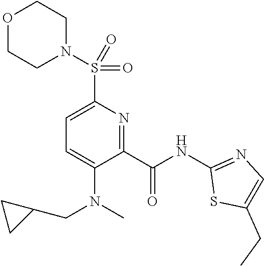 [SMILES: CCc1cnc(NC(=O)c2nc(S(=O)(=O)N3CCOCC3)ccc2N(C)CC2CC2)s1] | 7.921 | 33.3 |
| 30 | 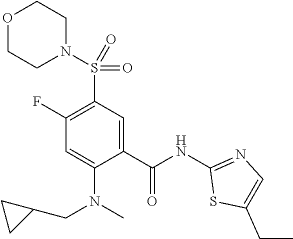 [SMILES: CCc1cnc(NC(=O)c2cc(S(=O)(=O)N3CCOCC3)c(F)cc2N(C)CC2CC2)s1] | 0.371 | 96.4 |
| 31 | 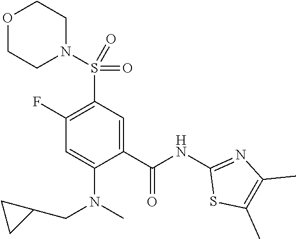 [SMILES: Cc1nc(NC(=O)c2cc(S(=O)(=O)N3CCOCC3)c(F)cc2N(C)CC2CC2)sc1C] | 0.431 | 61.2 |
| 33 | 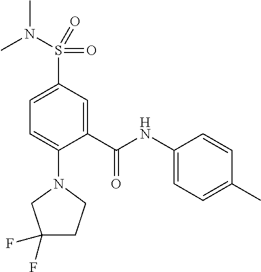 | 5.836 | 67.49 |
| 34 | 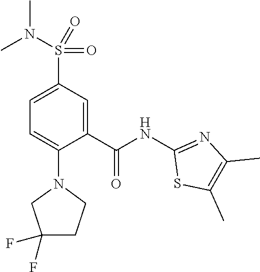 | 0.738 | 76.9 |
| 35 | 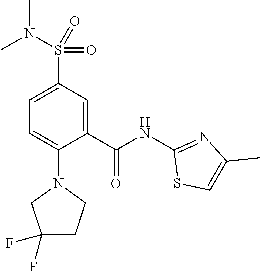 | 2.995 | 70 |
| 36 | 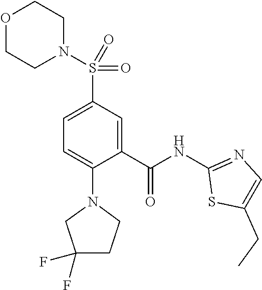 [SMILES: CCc1cnc(NC(=O)c2cc(S(=O)(=O)N3CCOCC3)ccc2N2CCC(F)(F)C2)s1] | 0.591 | 136.6 |
| 37 | 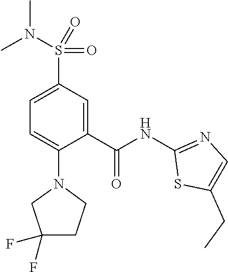 [SMILES: CCc1cnc(NC(=O)c2cc(S(=O)(=O)N(C)C)ccc2N2CCC(F)(F)C2)s1] | 0.5654 | 126 |
| 38 | 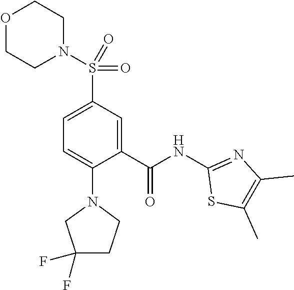 [SMILES: Cc1nc(NC(=O)c2cc(S(=O)(=O)N3CCOCC3)ccc2N2CCC(F)(F)C2)sc1C] | 0.7483 | 42.8 |

### ENUMERATED EMBODIMENTS

The following exemplary embodiments are provided, the numbering of which is not to be construed as designating levels of importance:

Embodiment 1 provides a compound of formula (I) or formula (II), or a salt, solvate, enantiomer, diastereomer, or tautomer thereof:

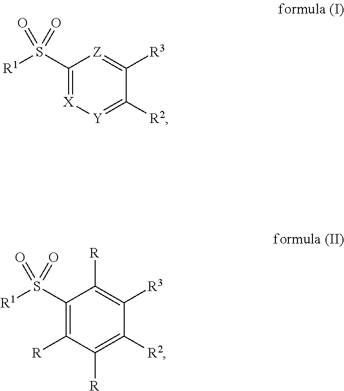

wherein:
X is CR⁴ or N;
Y is CR⁴ or N;
Z is CR⁴ or N;
   wherein at least one of X, Y, and Z is N;
each occurrence of R is independently selected from the group consisting of H, —(C₁-C₆)alkyl, —(C₃-C₆)cycloalkyl, —(C₁-C₆)haloalkyl, —(C₁-C₆)alkoxy, —(C₃-C₁₀)heterocyclyl, —(C₁-C₆)heteroalkyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl,
   wherein at least one R is not H;
   wherein each occurrence of alkyl, heteroalkyl, cycloalkyl, heterocyclyl, aryl, or heteroaryl group is independently optionally substituted;
R¹ is selected from the group consisting of:

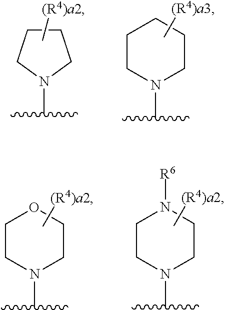

and —N(R⁴)₂;
R² is selected from the group consisting of:

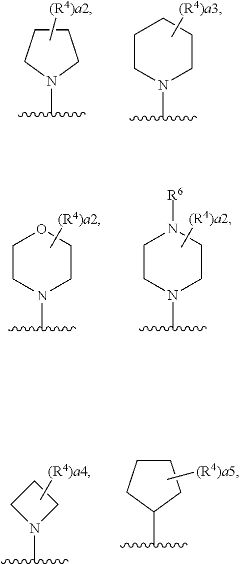

and —N(R⁴)₂;
R³ is selected from the group consisting of:

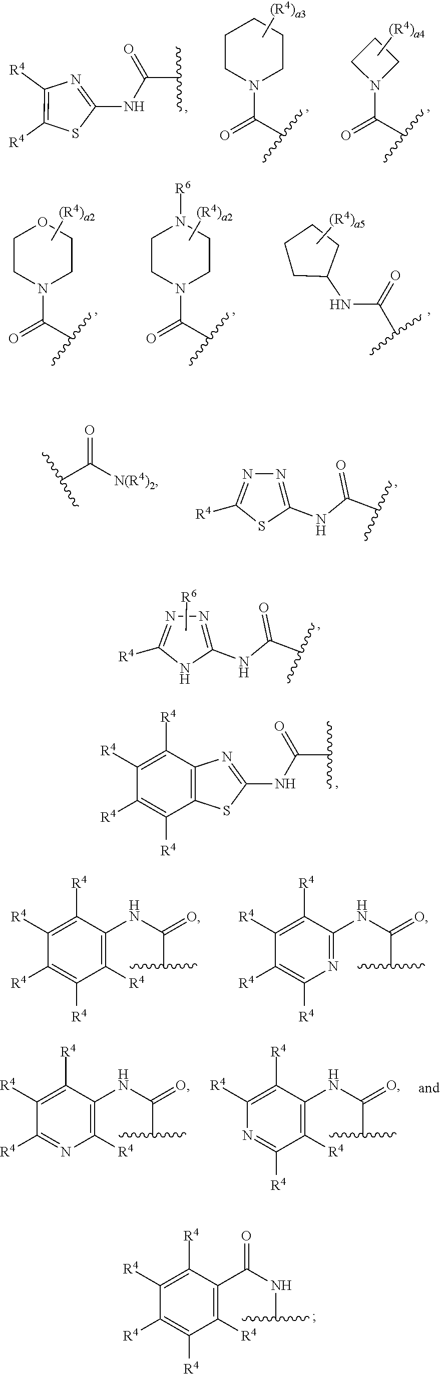

each occurrence of R⁴ is independently selected from the group consisting of H, —(C₁-C₅)alkyl, —(C₃-C₆)cycloalkyl, —(C₁-C₆)haloalkyl, —(C₁-C₆)alkoxy, —(C₁-C₆)heteroalkyl, —(C₃-C₁₀)heterocyclyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl, or two R⁴ bound to the same carbon atom or bound to adjacent carbon atoms combine to form a 3- to 10-membered cycloalkyl or heterocyclyl;
   wherein each occurrence of alkyl, heteroalkyl, cycloalkyl, heterocyclyl, aryl, or heteroaryl group is independently optionally substituted;
each occurrence of R⁵ is independently selected from the group consisting of H, —(C₁-C₆)alkyl, —(C₁-C₆)heteroalkyl, —(C₃-C₆)cycloalkyl, —(C₃-C₁₀)heterocyclyl, aryl, and —(C₅-C₁₀)heteroaryl,
   wherein each occurrence of alkyl, heteroalkyl, cycloalkyl, heterocyclyl, aryl, or heteroaryl group is independently optionally substituted;
each occurrence of R⁶ is independently selected from the group consisting of H, optionally substituted (C₁-C₆)alkyl, optionally substituted (C₁-C₆)heteroalkyl, optionally substituted (C₃-C₆)cycloalkyl, optionally substituted (C₁-C₆)acyl, and optionally substituted benzoyl;
each occurrence of a1 is independently 1, 2, or 3;
each occurrence of a2 is independently 1, 2, 3, 4, 5, 6, 7, or 8;
each occurrence of a3 is independently 1, 2, 3, 4, 5, 6, 7, 8, 9, or 10;
each occurrence of a4 is independently 1, 2, 3, 4, 5, or 6; and
each occurrence of a5 is independently 1, 2, 3, 4, 5, 6, 7, 8, or 9.

Embodiment 2 provides the compound of Embodiment 1, having the formula:

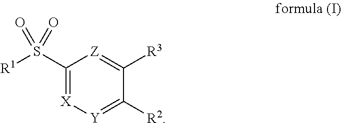

Embodiment 3 provides the compound of Embodiment 1, having the formula:

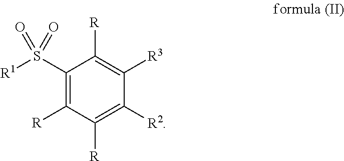

Embodiment 4 provides the compound of any of Embodiments 1-2, wherein one of the following applies:
X is N, and Y and Z are independently CR⁴;
Y is N, and X and Z are independently CR⁴;
Z is N, and X and Y are independently CR⁴;
X and Y are N, and Z is CR⁴;
Y and Z are N, and X is CR⁴;
X and Z are N, and Y is CR⁴; or
X, Y, and Z are N.

Embodiment 5 provides the compound of any of Embodiments 1-2 and 4, wherein R² is N(R⁴)(CH₃).

Embodiment 6 provides the compound of any of Embodiments 1-2 and 4-5, wherein at least one R⁴ in R² is —CH₂-(cyclopropyl) or cyclopentyl.

Embodiment 7 provides the compound of any of Embodiments 1-2 and 4-6, wherein R¹ is N(R⁴)₂ or

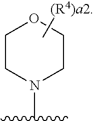

Embodiment 8 provides the compound of any of Embodiments 1-2 and 4-7, wherein R³ is

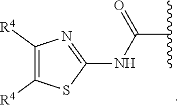

Embodiment 9 provides the compound of any of Embodiments 1-2 and 4-8, wherein each R⁴ in R³ is independently H or —(C₁-C₆)alkyl.

Embodiment 10 provides the compound of any of Embodiments 1-2 and 4-9, which is selected from the group consisting of:

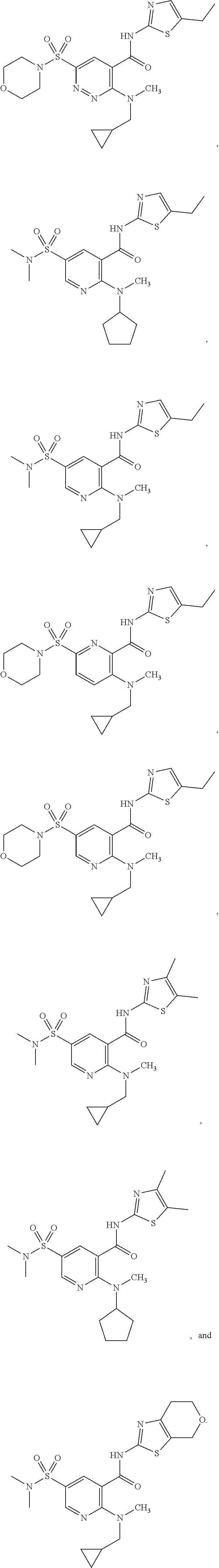

Embodiment 11 provides the compound of Embodiment 1 or 3, wherein at least one R is F, Cl, Br, or I.

Embodiment 12 provides the compound of any Embodiments 1, 3, and 11, wherein at least one R is F.

Embodiment 13 provides the compound of any of Embodiments 1, 3, and 11-12, wherein R¹ is N(R⁴)₂ or

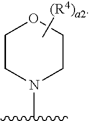

Embodiment 14 provides the compound of any of Embodiments 1, 3, and 11-13, wherein R² is —N(R⁴)(CH₃),

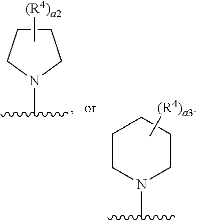

Embodiment 15 provides the compound of Embodiment 14, wherein R⁴ in —N(R⁴)(CH₃) is —CH₂-(cyclopropyl) or cyclopentyl.

Embodiment 16 provides the compound of any of Embodiments 1, 3, and 11-15, wherein R³ is

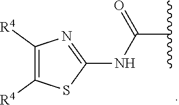

Embodiment 17 provides the compound of any of Embodiments 1, 3, and 11-16, wherein each R⁴ in R³ is independently H or —(C₁-C₆)alkyl.

Embodiment 18 provides the compound of any of Embodiments 1, 3, and 11-17, which is selected from the group consisting of:

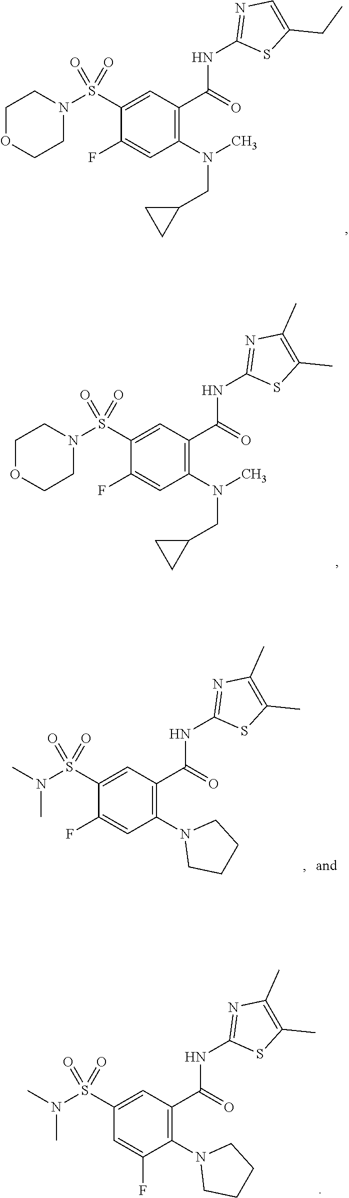

Embodiment 19 provides the compound of Embodiment 1 or 3, wherein in formula (II) R² is

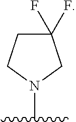 [SMILES: *N1CCC(F)(F)C1]

Embodiment 20 provides the compound of an of Embodiments 1, 3, and 19, wherein in formula (II) R³ is

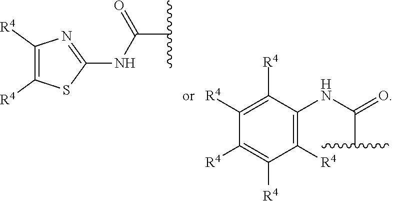

Embodiment 21 provides the compound of any of Embodiments 19-20, which is selected from the group consisting of:

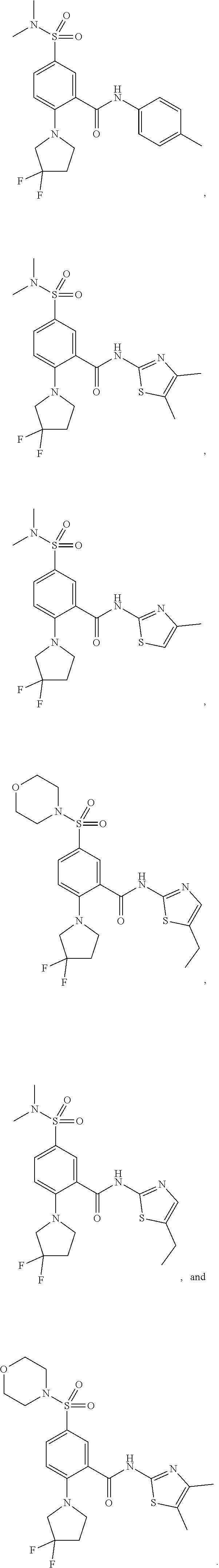

Embodiment 22 provides the compound of any of Embodiments 1-21, which preferentially inhibits the STT3B subunit as compared to the STT3A subunit.

Embodiment 23 provides a compound of formula (III), or a salt, solvate, enantiomer, diastereomer or tautomer thereof:

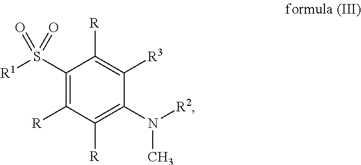

wherein:
each occurrence of R is independently selected from the group consisting of H, —(C₁-C₆)alkyl, —(C₃-C₆)cycloalkyl, —(C₁-C₆)haloalkyl, —(C₁-C₆)alkoxy, —(C₃-C₁₀)heterocyclyl, —(C₁-C₆)heteroalkyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl,
   wherein the alkyl, heteroalkyl, cycloalkyl, heterocyclyl, aryl, or heteroaryl group is optionally substituted;
R¹ is selected from the group consisting of

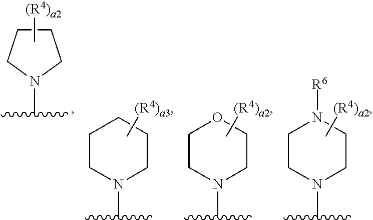

and —N(R⁴)₂;
R² is selected from the group consisting of —(C₁-C₆)alkyl, —(C₃-C₆)cycloalkyl, —(C₁-C₆)haloalkyl, —(C₃-C₁₀)heterocyclyl, —(C₁-C₆)heteroalkyl, aryl, —CH₂-aryl, and —(C₅-C₁₀)heteroaryl,
   wherein the alkyl, heteroalkyl, cycloalkyl, heterocyclyl, aryl, or heteroaryl group is optionally substituted;
R³ is selected from the group consisting of:

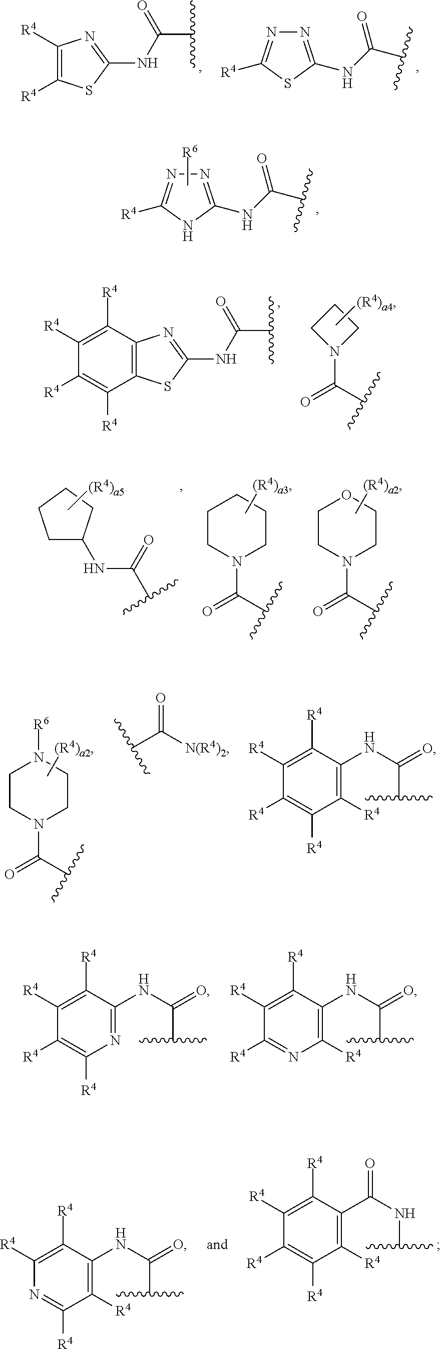

each occurrence of R⁴ is independently selected from the group consisting of H, —(C₁-C₆)alkyl, —(C₃-C₆)cycloalkyl, —(C₁-C₆)haloalkyl, —(C₁-C₆)alkoxy, —(C₃-C₁₀)heterocyclyl, —(C₁-C₆)heteroalkyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl, or two R⁴ bound to the same carbon atom or bound to adjacent carbon atoms combine to form a 3- to 10-membered cycloalkyl or heterocyclyl;
   wherein the alkyl, heteroalkyl, cycloalkyl, heterocyclyl, aryl, or heteroaryl group is optionally substituted;
each occurrence of R⁵ is independently selected from the group consisting of H, —(C₁-C₆)alkyl, —(C₁-C₆)heteroalkyl, —(C₃-C₆)cycloalkyl, —(C₃-C₁₀)heterocyclyl, aryl, and —(C₅-C₁₀)heteroaryl,
   wherein the alkyl, heteroalkyl, cycloalkyl, heterocyclyl, aryl, or heteroaryl group is optionally substituted;
each occurrence of R⁶ is independently selected from the group consisting of H, optionally substituted (C₁-C₆)alkyl, optionally substituted (C₁-C₆)heteroalkyl, optionally substituted (C₃-C₆)cycloalkyl, optionally substituted (C₁-C₆)acyl, and optionally substituted benzoyl.
each occurrence of a1 is independently 1, 2, or 3;
each occurrence of a2 is independently 1, 2, 3, 4, 5, 6, 7, or 8;
each occurrence of a3 is independently 1, 2, 3, 4, 5, 6, 7, 8, 9, or 10;
each occurrence of a4 is independently 1, 2, 3, 4, 5, or 6; and
each occurrence of a5 is independently 1, 2, 3, 4, 5, 6, 7, 8, or 9;
   with the proviso that the compound is not 2-(dimethylamino)-5-(dimethylsulfamoyl)-N-(5-methyl-1,3-thiazol-2-yl)benzamide.

Embodiment 24 provides the compound of Embodiment 23, wherein R² is CH₂-(cyclopropyl), —CH₂-(cyclobutyl), —CH₂-(cyclopentyl), —CH₂-(cyclohexyl), cyclobutyl, cyclopentyl, cyclohexyl, ethyl, isopropyl, isobutyl, or allyl.

Embodiment 25 provides the compound of any of Embodiments 23-24, wherein R¹ is N(R⁴)₂ or

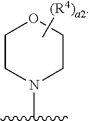

Embodiment 26 provides the compound of any of Embodiments 23-25, wherein each occurrence of R⁴ in R¹ is independently H or methyl.

Embodiment 27 provides the compound of any of Embodiments 23-26, wherein R³ is

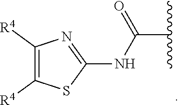

Embodiment 28 provides the compound of Embodiment 27, wherein each occurrence of R⁴ in R³ is independently H or —(C₁-C₆)alkyl.

Embodiment 29 provides the compound of any of Embodiments 23-28, wherein the compound is selected from the group consisting of:

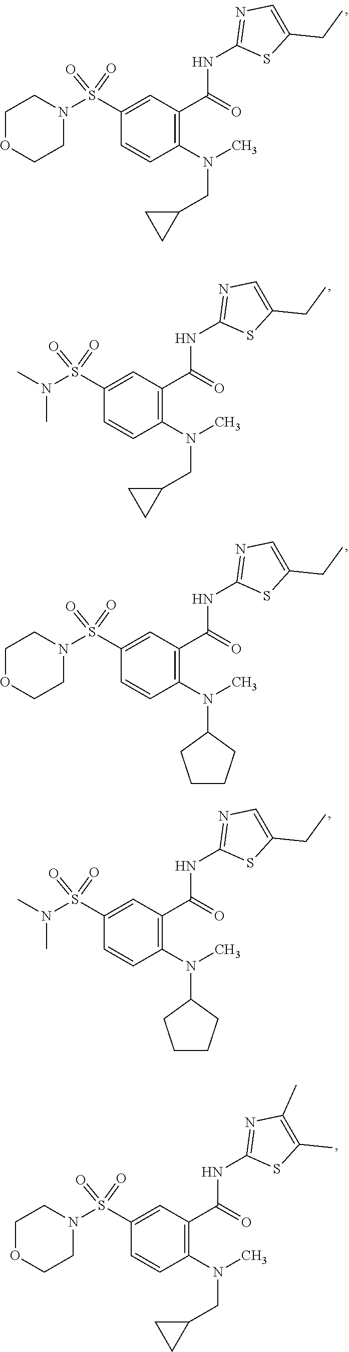

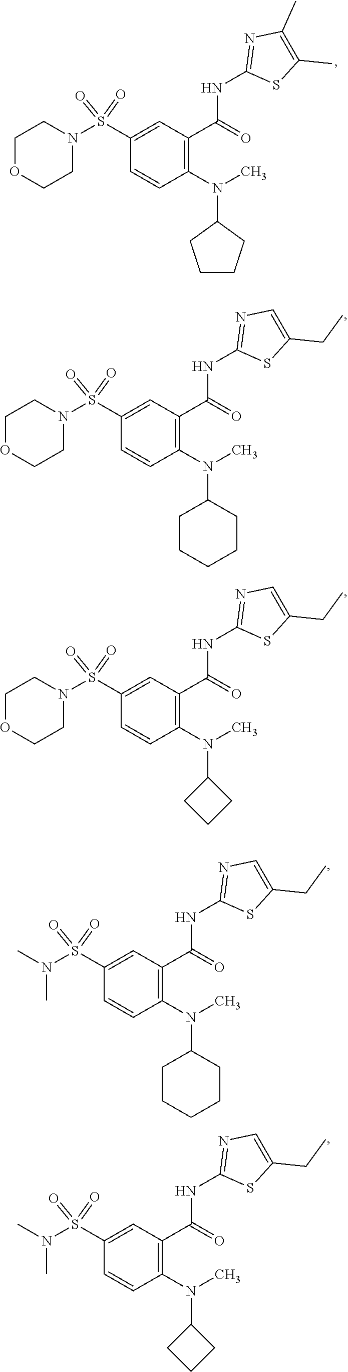

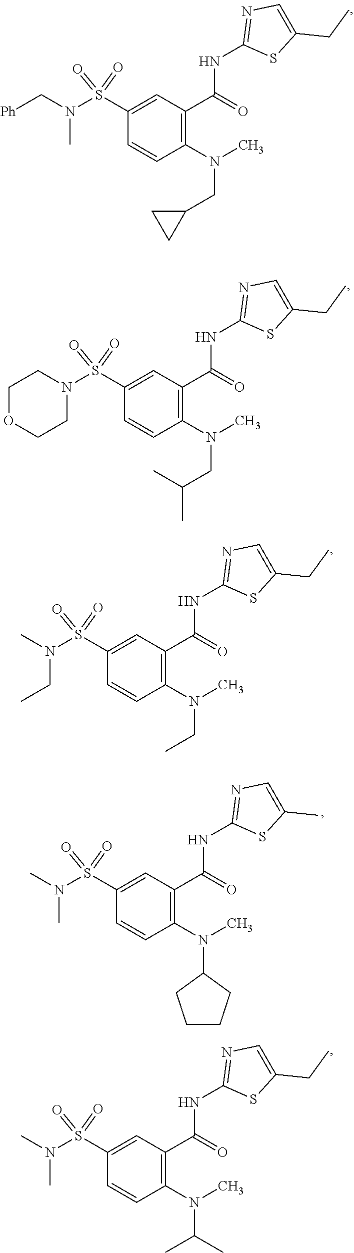

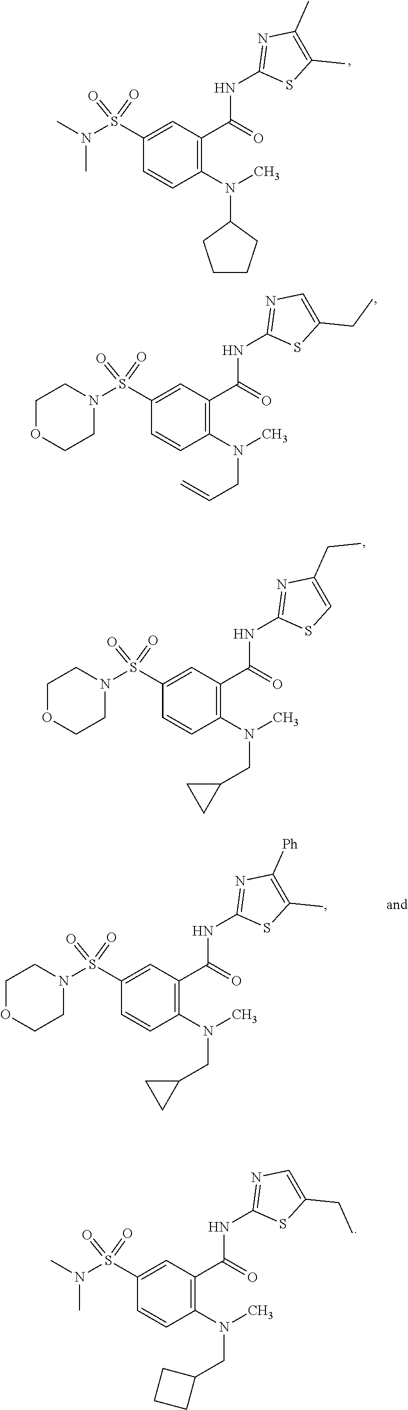

Embodiment 30 provides a pharmaceutical composition comprising at least one pharmaceutically acceptable carrier and at least one compound of any one of Embodiments 1-29.

Embodiment 31 provides the pharmaceutical composition of Embodiment 30, further comprising at least one additional therapeutic agent that treats or prevents cancer.

Embodiment 32 provides a method of inhibiting or disrupting N-linked glycosylation in a cell, the method comprising contacting the cell with an effective amount of a compound of any one of Embodiments 1-29 or the pharmaceutical composition of any one of Embodiments 30-31.

Embodiment 33 provides the method of Embodiment 32, wherein the compound inhibits or disrupts at least one cell process selected from the group consisting of oligosaccharyltransferase function and receptor tyrosine kinase cell surface expression.

Embodiment 34 provides the method of any of Embodiments 32-33, wherein the cell is a receptor tyrosine kinase-dependent cancer cell.

Embodiment 35 provides the method of Embodiment 34, wherein the cancer comprises at least one selected from the group consisting of non-small cell lung cancer, small cell lung cancer, head and neck squamous cell carcinoma, breast cancer, gastric cancer, cervical cancer, colon cancer, and glioma.

Embodiment 36 provides the method of any of Embodiments 32-35, wherein the compound causes at least one cellular effect selected from the group consisting of cell cycle arrest and/or senescence, and cell proliferation blockage or inhibition.

Embodiment 37 provides the method of any of Embodiments 32-36, wherein the cell is in vivo in a mammal.

Embodiment 38 provides the method of Embodiment 37, wherein the agent is administered to the mammal.

Embodiment 39 provides a method of preventing or treating a cancer in a subject in need thereof, the method comprising administering to the subject a therapeutically effective amount of at least one compound of any one of Embodiments 1-29 or the pharmaceutical composition of any one of Embodiments 30-31.

Embodiment 40 provides the method of Embodiment 39, wherein the compound inhibits or disrupts in a cell from the cancer at least one process selected from the group consisting of: N-linked glycosylation and oligosaccharyl transferase function.

Embodiment 41 provides the method of any of Embodiments 39-40, wherein the cancer is receptor tyrosine kinase-dependent.

Embodiment 42 provides the method of any of Embodiments 39-41, wherein the cancer comprises at least one selected from the group consisting of squamous cell cancer, small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer, pancreatic cancer, glioma, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney cancer, renal cancer, prostate cancer, hepatic carcinoma, anal carcinoma, penile carcinoma, and head and neck cancer.

Embodiment 43 provides the method of any of Embodiments 39-42, wherein the cancer comprises at least one selected from the group consisting of non-small cell lung cancer, small cell lung cancer, head and neck squamous cell carcinoma, breast cancer, gastric cancer, cervical cancer, colon cancer, and glioma.

Embodiment 44 provides the method of any of Embodiments 39-43, wherein the compound blocks or inhibits cell surface expression of the receptor tyrosine kinase in a cell from the cancer.

Embodiment 45 provides the method of any of Embodiments 39-44, wherein the compound causes at least one cellular effect selected from the group consisting of cell cycle arrest and/or senescence, and cell proliferation blockage or inhibition.

Embodiment 46 provides the method of any of Embodiments 39-45, further comprising administering to the subject at least one additional therapeutic agent that treats or prevents cancer.

Embodiment 47 provides the method of Embodiment 46, wherein the compound and the at least one additional therapeutic agent are co-administered to the subject.

Embodiment 48 provides the method of Embodiment 46, wherein the compound and the at least one additional therapeutic are agent coformulated.

Embodiment 49 provides the method of any of Embodiments 39-48, wherein the compound is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.

Embodiment 50 provides the method of any of Embodiments 39-49, wherein the subject is human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound of formula (I) or formula (II), or a salt, solvate, enantiomer, diastereomer, or tautomer thereof: 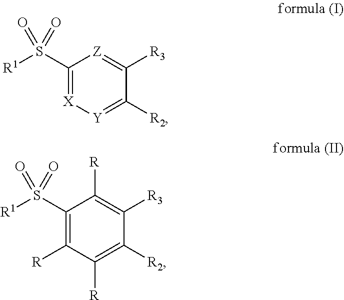
wherein:
X is CR⁴ or N;
Y is CR⁴ or N;
Z is CR⁴ or N;
wherein at least one of X, Y, and Z, if present, is N;
each occurrence of R is independently selected from the group consisting of H, —(C₁-C₆)alkyl, —(C₃-C₆)cycloalkyl, —(C₁-C₆)haloalkyl, —(C₁-C₆)alkoxy, —(C₃-C₁₀)heterocyclyl, —(C₁-C₆)heteroalkyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl,
wherein at least one R is not H;
wherein each occurrence of alkyl, heteroalkyl, cycloalkyl, heterocyclyl, aryl, or heteroaryl group is independently optionally substituted;
R¹ is selected from the group consisting of: 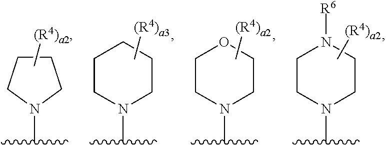
and —N(R⁴)₂;
R² is selected from the group consisting of: 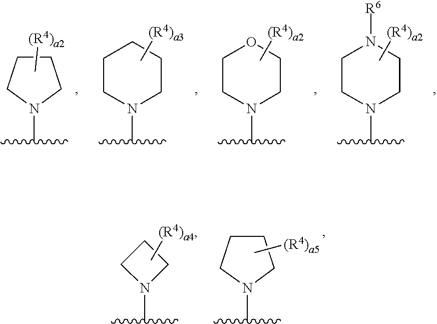
and —N(R⁴)₂;
R³ is selected from the group consisting of: 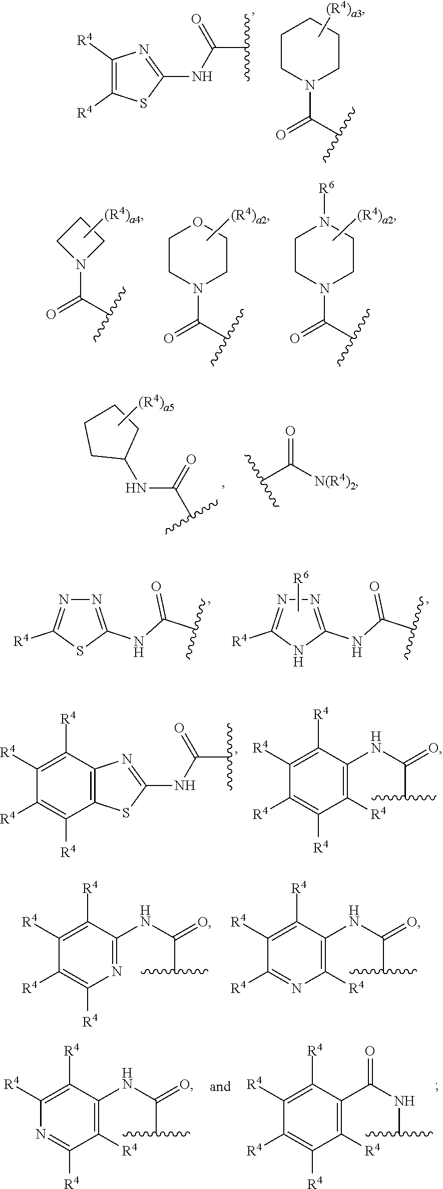
each occurrence of R⁴ is independently selected from the group consisting of H, —(C₁-C₆)alkyl, —(C₃-C₆)heteroalkyl, —(C₁-C₆)haloalkyl, —(C₁-C₆)alkoxy, —(C₁-C₆)heteroalkyl, —(C₃-C₁₀)heterocycyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl, or two R⁴ bound to the same carbon atom or bound to adjacent carbon atoms combine to form a 3- 10-membered cycloalkyl or heterocyclyl;
wherein each occurrence of alkyl, heteroalkyl, cycloalkyl, heterocyclyl, aryl, or heteroaryl group is independently optionally substituted;
each occurrence of R⁵ is independently selected from the group consisting of H, —(C₁-C₆)alkyl, —(C₃-C₆)heteroalkyl, —(C₃-C₆)cycloalkyl, —(C₃-C₁₀)heteroalkyl, aryl, and —(C₅-C₁₀)heterocycyl,
wherein each occurrence of alkyl, heteroalkyl, cycloakyl, heterocyclyl, aryl, or heteroaryl group is independently optionally substituted;
each occurrence of R⁶ is independently selected from the group consisting of H, optionally substituted (C₁-C₆)alkyl, optionally substituted (C₁-C₆)heteroalkyl, optionally substituted (C₃-C₆)cycloalkyl, optionally substituted (C₁-C₆)acyl, and optionally substituted benzoyl;
each occurrence of a2 is independently 1, 2, 3, 4, 5, 6, 7, or 8;
each occurrence of a3 is independently 1, 2, 3, 4, 5, 6, 7, 8, 9, or 10;
each occurrence of a4 is independently 1, 2, 3, 4, 5, or 6; and
each occurrence of a5 is independently 1, 2, 3, 4, 5, 6, 7, 8, or 9.

2. The compound of claim 1, having the formula:

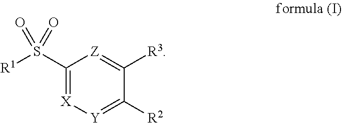

3. The compound of claim 1, having the formula:

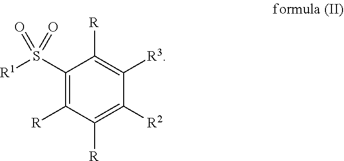

4. The compound of claim 2, wherein one of the following applies:
X is N, and Y and Z are independently CR⁴;
Y is N, and X and Z are independently CR⁴;
Z is N, and X and Y are independently CR⁴;
X and Y are N, and Z is CR⁴;
Y and Z are N, and X is CR⁴;
X and Z are N, and Y is CR⁴; or
X, Y, and Z are N.

5. The compound of claim 2, wherein at least one of the following applies:
(a)R² is N(R⁴)(CH₃);
(b) at least one R⁴ in R² is —CH₂-(cyclopropyl) or cyclopentyl;
(c) R¹ is N(R⁴)₂ or 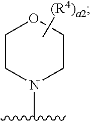
(d) R³ is 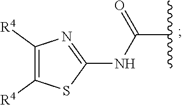
(e) each R⁴ in R³ is independently H or —(C₁-C₆)alkyl.

6-9. (canceled)

10. The compound of claim 2, which is selected from the group consisting of:

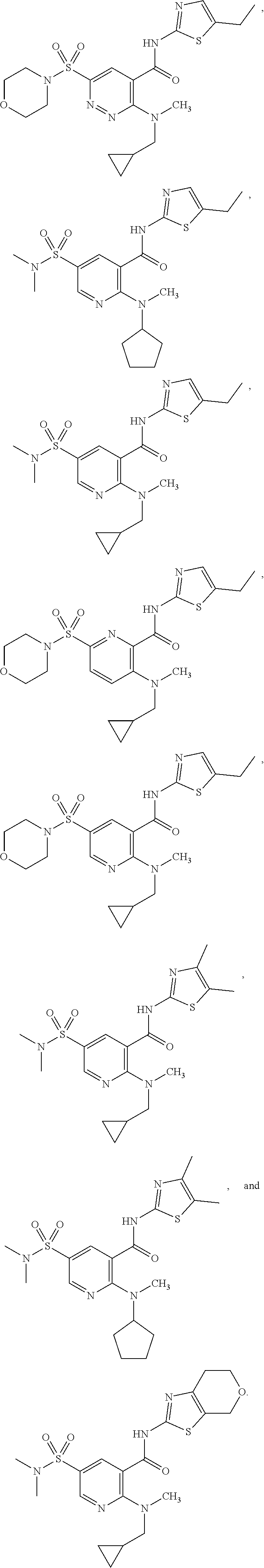

11. The compound of claim 3, wherein at least one of the following applies:
(a) R is F, Cl, Br, or I, optionally wherein R is F;
(b) R¹ is N(R⁴)₂ or 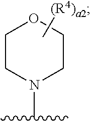
(c) R² is —N(R⁴)(CH₃), 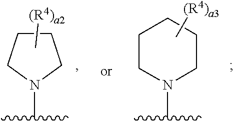
(d) R⁴ in —N(R⁴)(CH₃) is —CH₂-(cyclopropyl) or cyclopentyl;
(e) R³ is 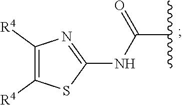
and
(f) each R⁴ in R³ is independently H or —(C₁-C₆)alkyl.

12-17. (canceled)

18. The compound of claim 3, which is selected from the group consisting of:

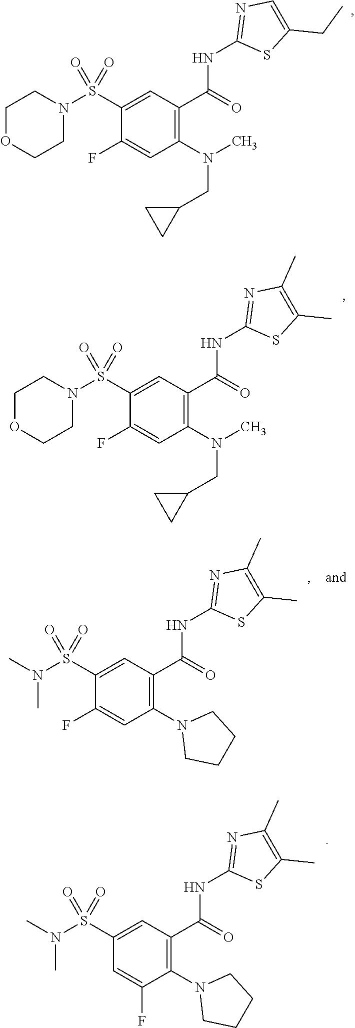

19. The compound of claim 1, wherein in formula (II), at least one of the following applies:
(a) R² is 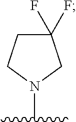 [SMILES: *N1CCC(F)(F)C1]
and
(b) R³ is

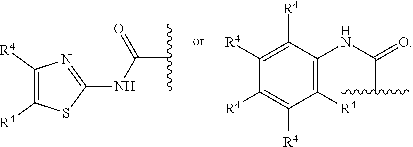

20. (canceled)

21. The compound of claim 19, which is selected from the group consisting of:

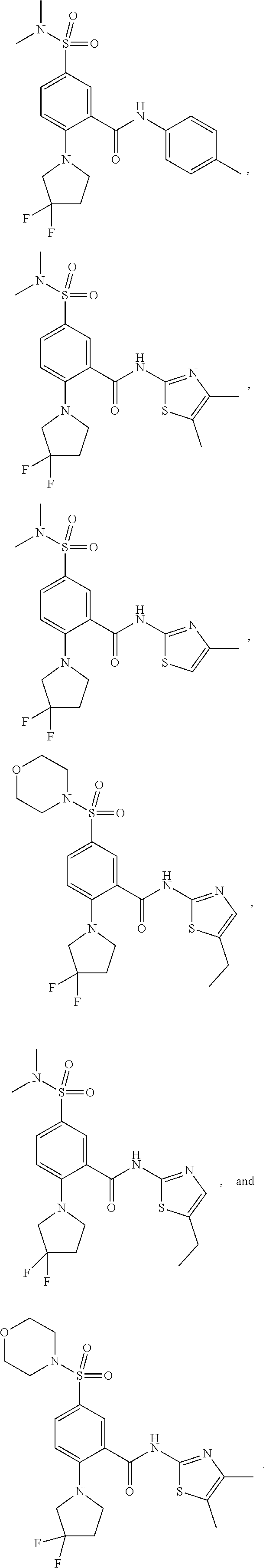

22. The compound of claim 1, which preferentially inhibits the STT3B subunit as compared to the STT3A subunit.

23. A compound of formula (III), or a salt, solvate, enantiomer, diastereomer or tautomer thereof: 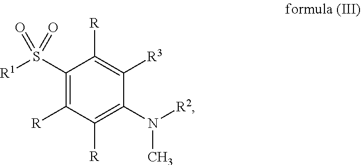
wherein:
each occurrence of R is independently selected from the group consisting of H, —(C₁-C₆)alkyl, —(C₃-C₆)cycloalkyl, —(C₁-C₆)haloalkyl, —(C₁-C₆)alkoxy, —(C₃-C₁₀)heterocyclyl, —(C₁-C₆)heteroalkyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl,
wherein the alkyl, heteroalkyl, cycloalkyl, heterocyclyl, aryl, or heteroaryl group is optionally substituted;
R¹ is selected from the group consisting of 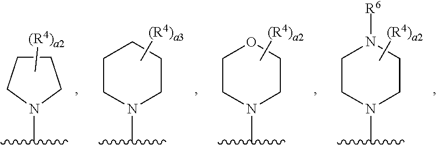
R² is selected from the group consisting of —(C₁-C₆)alkyl, —(C₃-C₆)cycloalkyl, —(C₁-C₆)haloalkyl, —(C₃-C₁₀)heterocyclyl, —(C₁-C₆)heteroalkyl, aryl, —CH₂-aryl, and —(C₅-C₁₀)heteroaryl,
wherein the alkyl, heteroalkyl, cycloalkyl, heterocyclyl, aryl, or heteroaryl group is optionally substituted;
R³ is selected from the group consisting of: 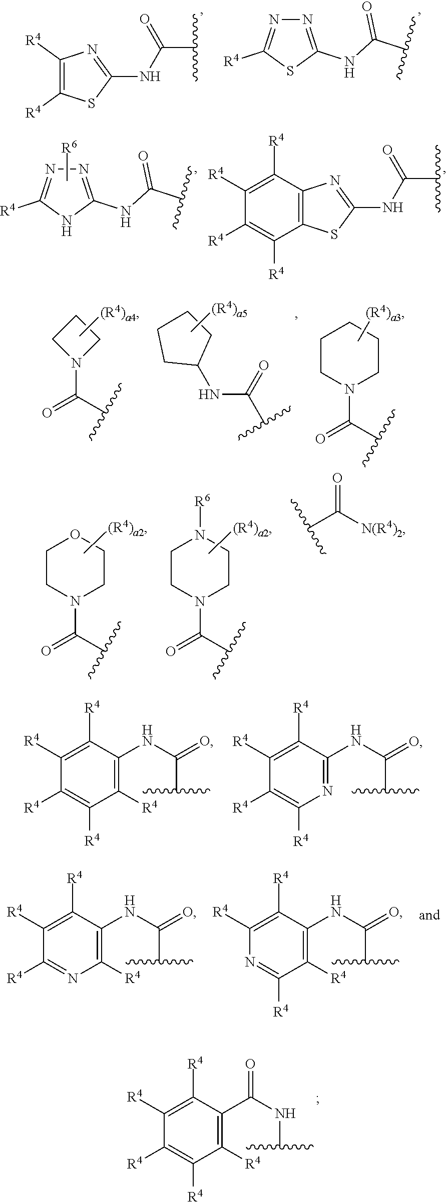
each occurrence of R⁴ is independently selected from the group consisting of H, —(C₁-C₆)alkyl, —(C₃-C₆)cycloalkyl, —(C₁-C₆)haloalkyl, —(C₁-C₆)alkoxy, —(C₃-C₁₀)heterocyclyl, —(C₁-C₆)heteroalkyl, —F, —Cl, —Br, —I, —CN, —NO₂, —OR⁵, —SR⁵, —S(═O)R⁵, —S(═O)₂R⁵, —C(═O)R⁵, —OC(═O)R⁵, —C(═O)OR⁵, aryl, —CH₂-aryl, and —(C₅-C₁₀)heteroaryl, or two R⁴ bound to the same carbon atom or bound to adjacent carbon atoms combine to form a 3- to 10-membered cycloalkyl or heterocyclyl;
wherein the alkyl, heteroalkyl, cycloalkyl, heterocyclyl, aryl, or heteroaryl group is optionally substituted;
each occurrence of R⁵ is independently selected from the group consisting of H, —(C₁-C₆)alkyl, —(C₁-C₆)heteroalkyl, —(C₃-C₆)cycloalkyl, —(C₃-C₁₀)heterocyclyl, aryl, and —(C₅-C₁₀)heteroaryl,
wherein the alkyl, heteroalkyl, cycloalkyl, heterocyclyl, aryl, or heteroaryl group is optionally substituted;
each occurrence of R⁶ is independently selected from the group consisting of H, optionally substituted (C₁-C₆)alkyl, optionally substituted (C₁-C₆)heteroalkyl, optionally substituted (C₃-C₆)cycloalkyl, optionally substituted (C₁-C₆)acyl, and optionally substituted benzoyl.
each occurrence of a2 is independently 1, 2, 3, 4, 5, 6, 7, or 8;
each occurrence of a3 is independently 1, 2, 3, 4, 5, 6, 7, 8, 9, or 10;
each occurrence of a4 is independently 1, 2, 3, 4, 5, or 6; and
each occurrence of a5 is independently 1, 2, 3, 4, 5, 6, 7, 8, or 9;
with the proviso that the compound is not 2-(dimethylamino)-5-(dimethylsulfamoyl)-N-(5-methyl-1,3-thiazol-2-yl)benzamide.

24. The compound of claim 23, wherein at least one of the following applies:
(a) R² is CH₂-(cyclopropyl), —CH₂-(cyclobutyl), —CH₂-(cyclopentyl), —CH₂-(cyclohexyl), cyclobutyl, cyclopentyl, cyclohexyl, ethyl, isopropyl, isobutyl, or allyl;
(b) R¹ is N(R⁴)₂ or 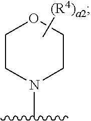
(c) each occurrence of R⁴ in R¹ is independently H or methyl;
(d) R³ is 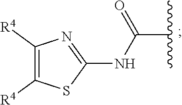
and
(e) each occurrence of R⁴ in R³ is independently H or —(C₁-C₆)alkyl.

25-28. (canceled)

29. The compound of claim 23, wherein the compound is selected from the group consisting of:

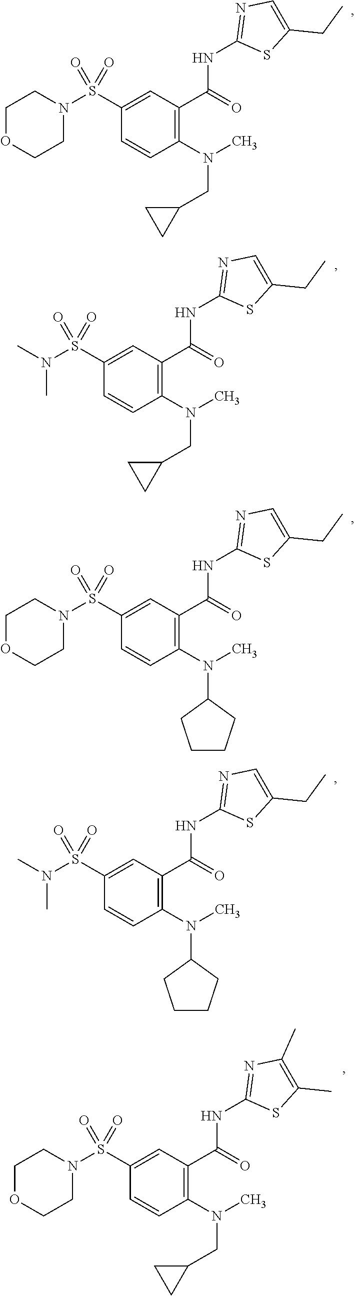

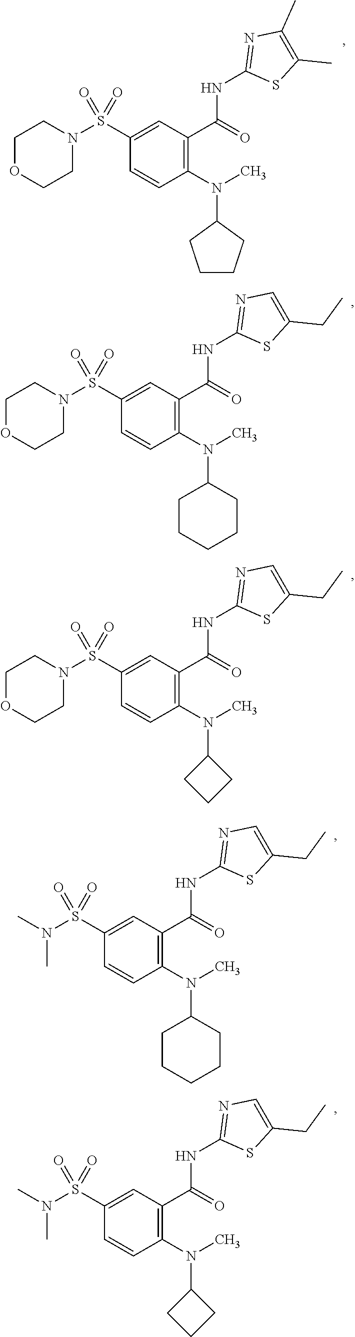

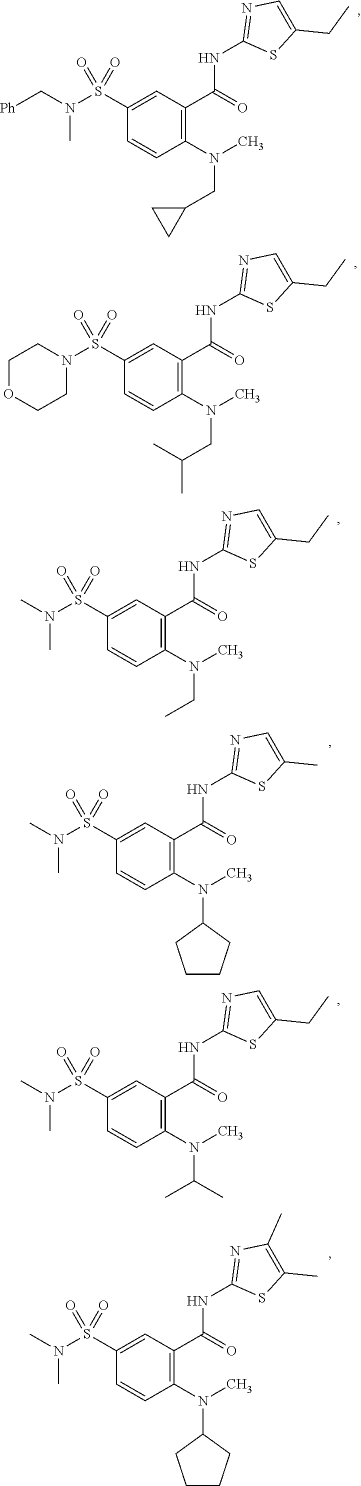

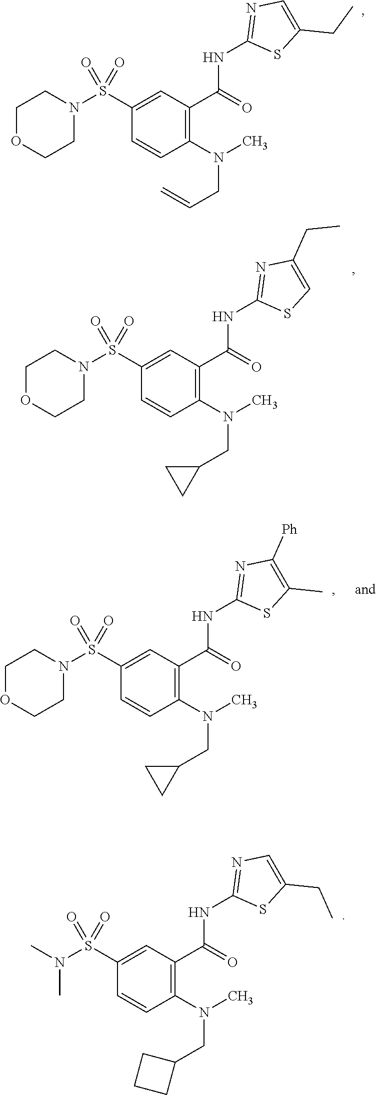

30. A pharmaceutical composition comprising at least one pharmaceutically acceptable carrier and at least one compound of claim 1, optionally wherein the composition further comprises at least one additional therapeutic agent that treats or prevents cancer.

31. (canceled)

32. A method of inhibiting or disrupting N-linked glycosylation in a cell, the method comprising contacting the cell with an effective amount of a compound of claim 1.

33. The method of claim 32, wherein at least one of the following applies:
(a) the compound inhibits or disrupts at least one cell process selected from the group consisting of oligosaccharyltransferase function and receptor tyrosine kinase cell surface expression;
(b) the cell is a receptor tyrosine kinase-dependent cancer cell,
optionally wherein the cancer cell comprises at least one selected from the group consisting of non-small cell lung cancer, small cell lung cancer, head and neck squamous cell carcinoma, breast cancer, gastric cancer, cervical cancer, colon cancer, and glioma;
(c) wherein the compound causes at least one cellular effect selected from the group consisting of cell cycle arrest and/or senescence, and cell proliferation blockage or inhibition; and
(d) wherein the cell is in vivo in a mammal,
optionally wherein the agent is administered to the mammal.

34-38. (canceled)

39. A method of preventing or treating a cancer in a subject in need thereof, the method comprising administering to the subject a therapeutically effective amount of at least one compound of claim 1.

40. The method of claim 39, wherein at least one of the following applies:
(a) the compound inhibits or disrupts in a cell from the cancer at least one process selected from the group consisting of: N-linked glycosylation and oligosaccharyl transferase function;
(b) the cancer is receptor tyrosine kinase-dependent,
optionally wherein the cancer comprises at least one selected from the group consisting of squamous cell cancer, small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer, pancreatic cancer, glioma, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney cancer, renal cancer, prostate cancer, hepatic carcinoma, anal carcinoma, penile carcinoma, and head and neck cancer;
(c) the compound blocks or inhibits cell surface expression of the receptor tyrosine kinase in a cell from the cancer;
(d) the compound causes at least one cellular effect selected from the group consisting of cell cycle arrest and/or senescence, and cell proliferation blockage or inhibition;
(e) the compound is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes; and
(f) the subject is a human.

41-45. (canceled)

46. The method of claim 39, further comprising administering to the subject at least one additional therapeutic agent that treats or prevents cancer, optionally wherein the compound and the at least one additional therapeutic agent are co-administered to the subject, and optionally wherein the compound and the at least one therapeutic agent are coformulated.

47-50. (canceled)
